SEQUENCE LISTING (142 sequences)

SEQ ID NO: 1 - protein, 117 aa, Artificial Sequence
  Synthetic Construct
QLLFNKTKSVEFTFSNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVS

SEQ ID NO: 2 - protein, 120 aa, Artificial Sequence
  Synthetic Construct
WQLPLLFNKTKSVEFTFGNDTVVIPCFVTNMEAQNTTEVYVKWKFKGRDIYTFDGDKNKSTVPTDFSSAKIEVSQLLKGDASLKMDKSDAVSHTGNYTCEVTELTREGETIIELKYRVVS

SEQ ID NO: 3 - protein, 120 aa, Artificial Sequence
  Synthetic Construct
WQPPLLFNKTKSVEFTFGNDTVVIPCFVTNMEAQNTTEVYVKWKFKGRDIYTFDGQANKSTVPTDFSSAKIEVSQLLKGDASLKMDKSDAVSHTGNYTCEVTELTREGETIIELKYRVVS

SEQ ID NO: 4 - protein, 120 aa, Artificial Sequence
  Synthetic Construct
WQPPLLFNKTKSVEFTFCNDTVVIPCFVTNMEAQNTTEVYVKWKFKGRDIYTFDGQANKSTVPTDFSSAKIEVSQLLKGDASLKMDKSDAVSHTGNYTCEVTELTREGETIIELKYRVVS

SEQ ID NO: 5 - protein, 120 aa, Artificial Sequence
  Synthetic Construct
WQPPLLFNKTKSVEFTCGNDTVVIPCFVTNMEAQNTTEVYVKWKFKGRDIYTFDGQANKSTVPTDFSSAKIEVSQLLKGDASLKMDKSDAVSHTGNYTCEVTELTREGETIIELKYRVVS

SEQ ID NO: 6 - protein, 138 aa, Artificial Sequence
  Synthetic Construct
QLLFNKTKSVEFTFSNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSHHHHHHGLNDIFEAQKIEWHE

SEQ ID NO: 7 - protein, 6 aa, Artificial Sequence
  Synthetic Construct
HHHHHH

SEQ ID NO: 8 - protein, 15 aa, Artificial Sequence
  Synthetic Construct
GLNDIFEAQKIEWHE

SEQ ID NO: 9 - protein, 13 aa, Artificial Sequence
  Synthetic Construct
SRLEEELRRRLTE

SEQ ID NO: 10 - protein, 26 aa, Artificial Sequence
  Synthetic Construct
KRRWKKNFIAVSAANRFKKISSSGAL

SEQ ID NO: 11 - protein, 6 aa, Artificial Sequence
  Synthetic Construct
EEEEEE

SEQ ID NO: 12 - protein, 13 aa, Artificial Sequence
  Synthetic Construct
GAPVPYPDPLEPR

SEQ ID NO: 13 - protein, 8 aa, Artificial Sequence
  Synthetic Construct
DYKDDDDK

SEQ ID NO: 14 - protein, 9 aa, Artificial Sequence
  Synthetic Construct
YPYDVPDYA

SEQ ID NO: 15 - protein, 18 aa, Artificial Sequence
  Synthetic Construct
TKENPRSNQEESYDDNES

SEQ ID NO: 16 - protein, 9 aa, Artificial Sequence
  Synthetic Construct
TETSQVAPA

SEQ ID NO: 17 - protein, 15 aa, Artificial Sequence
  Synthetic Construct
KETAAAKFERQHMDS

SEQ ID NO: 18 - protein, 38 aa, Artificial Sequence
  Synthetic Construct
MDEKTTGWRGGHVVEGLAGELEQLRARLEHHPQGQREP

SEQ ID NO: 19 - protein, 13 aa, Artificial Sequence
  Synthetic Construct
SLAELLNAGLGGS

SEQ ID NO: 20 - protein, 8 aa, Artificial Sequence
  Synthetic Construct
TQDPSRVG

SEQ ID NO: 21 - protein, 8 aa, Artificial Sequence
  Synthetic Construct
WSHPQFEK

SEQ ID NO: 22 - protein, 11 aa, Artificial Sequence
  Synthetic Construct
MASMTGGQQMG

SEQ ID NO: 23 - protein, 10 aa, Artificial Sequence
  Synthetic Construct
EVHTNQDPLD

SEQ ID NO: 24 - protein, 14 aa, Artificial Sequence
  Synthetic Construct
GKPIPNPLLGLDST

SEQ ID NO: 25 - protein, 11 aa, Artificial Sequence
  Synthetic Construct
YTDIEMNRLGK

SEQ ID NO: 26 - protein, 8 aa, Artificial Sequence
  Synthetic Construct
DLYDDDDK

SEQ ID NO: 27 - protein, 16 aa, Artificial Sequence
  Synthetic Construct
TDKDMTITFTNKKDAE

SEQ ID NO: 28 - protein, 13 aa, Artificial Sequence
  Synthetic Construct
AHIVMVDAYKPTK

SEQ ID NO: 29 - protein, 12 aa, Artificial Sequence
  Synthetic Construct
KLGDIEFIKVNK

SEQ ID NO: 30 - protein, 12 aa, Artificial Sequence
  Synthetic Construct
KLGSIEFIKVNK

SEQ ID NO: 31 - protein, 23 aa, Artificial Sequence
  Synthetic Construct
DIPATYEFTDGKHYITNEPIPPK

SEQ ID NO: 32 - protein, 13 aa, Artificial Sequence
  Synthetic Construct
DPIVMIDNDKPIT

SEQ ID NO: 33 - protein, 119 aa, Artificial Sequence
  Synthetic Construct
EEELQIIQPDKSVLVAAGETATLRCTITSLFPVGPIQWFRGAGPGRVLIYNQRQGPFPRVTTVSDTTKRNNMDFSIRIGAITPADAGTYYCIKFRKGSPDDVEFKSGAGTELSVRAKPS

SEQ ID NO: 34 - protein, 119 aa, Artificial Sequence
  Synthetic Construct
EEELQIIQPDKSVLVAAGETATLRCTITSLFPVGPIQWFRGAGPGRELIYNQREGPFPRVTTVSDTTKRNNMDFSIRIGAITPADAGTYYCVKFRKGSPDDVEFKSGAGTELSVRAKPS

SEQ ID NO: 35 - protein, 119 aa, Artificial Sequence
  Synthetic Construct
EEELQIIQPDKSVLVAAGETATLRCTITSLFPVGPIQWFRGAGPGRVLIYNQREGPFPRVTTVSDTTKRNNMDFSIRIGAITPADAGTYYCIKFRKGSPDDVEFKSGAGTELSVRAKPS

SEQ ID NO: 36 - protein, 119 aa, Artificial Sequence
  Synthetic Construct
EDELQIIQPEKSVSVAAGESATLRCAITSLFPVGPIQWFRGAGAGRVLIYNQRQGPFPRVTTVSETTKRNNLDFSISISNITPADAGTYYCIKFRKGSPDDVEFKSGAGTELSVRAKPS

SEQ ID NO: 37 - protein, 119 aa, Artificial Sequence
  Synthetic Construct
EEELQIIQPDKSISVAAGESATLHCTITSLFPVGPIQWFRGAGPGRVLIYNQRQGPFPRVTTVSDTTKRNNMDFSIRISNITPADAGTYYCIKFRKGSPDDVEFKSGAGTELSVRAKPS

SEQ ID NO: 38 - protein, 119 aa, Artificial Sequence
  Synthetic Construct
EEELQIIQPEKLLLVTVGKTATLHCTITSLFPVGPIQWFRGVGPGRVLIYNQRDGPFPRVTTVSDGTKRNNMDFSIRISSITPADVGTYYCVKFRKGTPEDVEFKSGPGTEMALGAKPS

SEQ ID NO: 39 - protein, 119 aa, Artificial Sequence
  Synthetic Construct
EEELQIIQPEKLLLVTVGKTATLHCTITSLFPVGPIQWFRGVGPGRVLIYNQKDGPFPRVTTVSDGTKRNNMDFSIRISSITPADVGTYYCVKFRKGSPEDVEFKSGPGTEMALGAKPS

SEQ ID NO: 40 - protein, 119 aa, Artificial Sequence
  Synthetic Construct
EEELQIIQPEKLLLVTVGKTATLHCTITSLFPVGPIQWFRGVGPGRVLIYNQKDGHFPRVTTVSDGTKRNNMDFSIRISSITPADVGTYYCVKFRKGSPEDVEFKSGPGTEMALGAKPS

SEQ ID NO: 41 - protein, 119 aa, Artificial Sequence
  Synthetic Construct
EEELQIIQPEKLLLVTVGKTATLHCTITSHFPVGPIQWFRGVGPGRVLIYNQKDGHFPRVTTVSDGTKRNNMDFSIRISSITPADVGTYYCVKFRKGSPEDVEFKSGPGTEMALGAKPS

SEQ ID NO: 42 - protein, 119 aa, Artificial Sequence
  Synthetic Construct
EEELQIIQPEKLLLVTVGKTATLHCTITSLFPVGPVLWFRGVGPGRVLIYNQRQGPFPRVTTVSDTTKRNNMDFSIRISSITPADVGTYYCVKFRKGTPEDVEFKSGPGTEMALGAKPS

SEQ ID NO: 43 - protein, 119 aa, Artificial Sequence
  Synthetic Construct
EEELQIIQPEKLLLVTVGKTATLHCTITSLFPVGPIQWFRGVGPGRELIYNAREGRFPRVTTVSDLTKRNNMDFSIRISSITPADVGTYYCVKFRKGSPEDVEFKSGPGTEMALGAKPS

SEQ ID NO: 44 - protein, 119 aa, Artificial Sequence
  Synthetic Construct
EEELQIIQPEKLLLVTVGKTATLHCTITSLLPVGPIQWFRGVGPGRELIYNQRDGPFPRVTTVSDGTKRNNMDFSIRISSITPADVGTYYCVKFRKGTPEDVEFKSGPGTEMALGAKPS

SEQ ID NO: 45 - protein, 119 aa, Artificial Sequence
  Synthetic Construct
EEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPS

SEQ ID NO: 46 - protein, 119 aa, Artificial Sequence
  Synthetic Construct
DVQLVESGGGVVRPGESLRLSCAASGFSFSSYAMNWVRQAPGEGLEWVSRINSGGGGTDYAESVKGRFTISRDNSENTLYLQMNSLRAEDTAVYYCAKQYDWNSFFDYWGLGALVTVSS

SEQ ID NO: 47 - protein, 108 aa, Artificial Sequence
  Synthetic Construct
ETVLTQSPATLSVSPGERATLSCRASQTVGSKLAWHQQKPGQAPRLLIYDATNRATGISDRFSGSGSGTDFTLTISSLQTEDSAVYYCQQYYYWPPYRFGGGTKVEIK

SEQ ID NO: 48 - protein, 117 aa, Artificial Sequence
  Synthetic Construct
DVQLVESGGGVVRPGESLRLSCEASGFTFSSNAMSWVRQAPGKGLEWVAGISSGSDTYYGDSVKGRLTISRDNSKNILYLQMNSLTAEDTAVYYCARETWNHLFDYWGQGTLVTVSS

SEQ ID NO: 49 - protein, 108 aa, Artificial Sequence
  Synthetic Construct
SYELTQPPSVSVSPGQTARITCSGGSYSSYYYAWYQQKPGQAPVTLIYSDDKRPSNIPERFSGSSSGTTVTLTISGVQAEDEADYYCGGYDQSSYTNPFGGGTKLTVL

SEQ ID NO: 50 - protein, 119 aa, Artificial Sequence
  Synthetic Construct
DVQLVESGGGVVRPGESLRLSCAASGFTFSSYDMNWVRQAPGEGLEWVSLISGSGEIIYYADSVKGRFTISRDNSKNTLYLQMNSLRAEDTAVYYCAKENNRYRFFDDWGQGTLVTVSS

SEQ ID NO: 51 - protein, 108 aa, Artificial Sequence
  Synthetic Construct
ETVLTQSPGTLTLSPGERATLTCRASQSVYTYLAWYQEKPGQAPRLLIYGASSRATGIPDRFSGSGSGTEFTLTISSLQSEDFAVYYCQQYYDRPPLTFGGGTKVEIK

SEQ ID NO: 52 - protein, 5 aa, Artificial Sequence
  Synthetic Construct
GGGGS

SEQ ID NO: 53 - protein, 4 aa, Artificial Sequence
  Synthetic Construct
GGSG

SEQ ID NO: 54 - protein, 4 aa, Artificial Sequence
  Synthetic Construct
SGGG

SEQ ID NO: 55 - protein, 4 aa, Artificial Sequence
  Synthetic Construct
GSGS

SEQ ID NO: 56 - protein, 6 aa, Artificial Sequence
  Synthetic Construct
GSGSGS

SEQ ID NO: 57 - protein, 8 aa, Artificial Sequence
  Synthetic Construct
GSGSGSGS

SEQ ID NO: 58 - protein, 10 aa, Artificial Sequence
  Synthetic Construct
GSGSGSGSGS

SEQ ID NO: 59 - protein, 12 aa, Artificial Sequence
  Synthetic Construct
GSGSGSGSGSGS

SEQ ID NO: 60 - protein, 6 aa, Artificial Sequence
  Synthetic Construct
GGSGGS

SEQ ID NO: 61 - protein, 9 aa, Artificial Sequence
  Synthetic Construct
GGSGGSGGS

SEQ ID NO: 62 - protein, 8 aa, Artificial Sequence
  Synthetic Construct
GGSGGGSG

SEQ ID NO: 63 - protein, 12 aa, Artificial Sequence
  Synthetic Construct
GGSGGGSGGGSG

SEQ ID NO: 64 - protein, 10 aa, Artificial Sequence
  Synthetic Construct
GENLYFQSGG

SEQ ID NO: 65 - protein, 8 aa, Artificial Sequence
  Synthetic Construct
SACYCELS

SEQ ID NO: 66 - protein, 5 aa, Artificial Sequence
  Synthetic Construct
RSIAT

SEQ ID NO: 67 - protein, 17 aa, Artificial Sequence
  Synthetic Construct
RPACKIPNDLKQKVMNH

SEQ ID NO: 68 - protein, 36 aa, Artificial Sequence
  Synthetic Construct
GGSAGGSGSGSSGGSSGASGTGTAGGTGSGSGTGSG

SEQ ID NO: 69 - protein, 17 aa, Artificial Sequence
  Synthetic Construct
AAANSSIDLISVPVDSR

SEQ ID NO: 70 - protein, 36 aa, Artificial Sequence
  Synthetic Construct
GGSGGGSEGGGSEGGGSEGGGSEGGGSEGGGSGGGS

SEQ ID NO: 71 - protein, 5 aa, Artificial Sequence
  Synthetic Construct
GVGVP

SEQ ID NO: 72 - protein, 6 aa, Artificial Sequence
  Synthetic Construct
APGVGV

SEQ ID NO: 73 - protein, 6 aa, Artificial Sequence
  Synthetic Construct
GAGAGS

SEQ ID NO: 74 - protein, 5 aa, Artificial Sequence
  Synthetic Construct
GPGGG

SEQ ID NO: 75 - protein, 7 aa, Artificial Sequence
  Synthetic Construct
GGYGPGS

SEQ ID NO: 76 - protein, 15 aa, Artificial Sequence
  Synthetic Construct
GAPGAPGSQGAPGLQ

SEQ ID NO: 77 - protein, 15 aa, Artificial Sequence
  Synthetic Construct
GAPGTPGPQGLPGSP

SEQ ID NO: 78 - protein, 13 aa, Artificial Sequence
  Synthetic Construct
AKLKLAEAKLELA

SEQ ID NO: 79 - protein, 30 aa, Artificial Sequence
  Synthetic Construct
PPAKVPEVPEPKKPVPEEKVPVPVPKKPEA

SEQ ID NO: 80 - protein, 10 aa, Artificial Sequence
  Synthetic Construct
  VARIANT 10: Xaa = Any Amino Acid
GGFGGMGGGX

SEQ ID NO: 81 - protein, 226 aa, Artificial Sequence
  Synthetic Construct
VPRDSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFIYSKLNVQKSNWEAGNTFTCSVLHEGLHNHHTEKSLSHSPG

SEQ ID NO: 82 - protein, 226 aa, Artificial Sequence
  Synthetic Construct
VPRDSGCKPCICTVPEVSSVFIFPPKPKDVLTITLTPKVTCVVVDISKDDPEVQFSWFVDDVEVHTAQTQPREEQFASTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFIYSKLNVQKSNWEAGNTFTCSVLHEGLHNHHTEKSLSHSPG

SEQ ID NO: 83 - protein, 232 aa, Artificial Sequence
  Synthetic Construct
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 84 - protein, 343 aa, Artificial Sequence
  Synthetic Construct
QLLFNKTKSVEFTFSNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSVPRDSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFIYSKLNVQKSNWEAGNTFTCSVLHEGLHNHHTEKSLSHSPG

SEQ ID NO: 85 - protein, 343 aa, Artificial Sequence
  Synthetic Construct
QLLFNKTKSVEFTFSNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSVPRDSGCKPCICTVPEVSSVFIFPPKPKDVLTITLTPKVTCVVVDISKDDPEVQFSWFVDDVEVHTAQTQPREEQFASTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFIYSKLNVQKSNWEAGNTFTCSVLHEGLHNHHTEKSLSHSPG

SEQ ID NO: 86 - protein, 349 aa, Artificial Sequence
  Synthetic Construct
QLLFNKTKSVEFTFSNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSEPRGPTIKPSPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 87 - protein, 8 aa, Artificial Sequence
  Synthetic Construct
  VARIANT 2, 3, 7: Xaa = Any Amino Acid
RXXRKVXG

SEQ ID NO: 88 - protein, 11 aa, Artificial Sequence
  Synthetic Construct
KRRKQGASRKA

SEQ ID NO: 89 - protein, 12 aa, Artificial Sequence
  Synthetic Construct
  VARIANT 4, 6, 7, 9: Xaa = Any Amino Acid
LSGXRXXSXDNH

SEQ ID NO: 90 - protein, 10 aa, Artificial Sequence
  Synthetic Construct
  VARIANT 1, 2, 3, 4, 5, 6, 8, 9, 10: Xaa = Any Amino Acid
XXXXXXNXXX

SEQ ID NO: 91 - protein, 5 aa, Artificial Sequence
  Synthetic Construct
  VARIANT 4: Xaa = Any Amino Acid
AANXL

SEQ ID NO: 92 - protein, 12 aa, Artificial Sequence
  Synthetic Construct
  VARIANT 5, 11, 12: Xaa = Any Amino Acid
SISQXYQRSSXX

SEQ ID NO: 93 - protein, 5 aa, Artificial Sequence
  Synthetic Construct
SSKLQ

SEQ ID NO: 94 - protein, 9 aa, Artificial Sequence
  Synthetic Construct
  VARIANT 1, 3, 4, 7, 8, 9: Xaa = Any Amino Acid
XPXXLIXXX

SEQ ID NO: 95 - protein, 9 aa, Artificial Sequence
  Synthetic Construct
  VARIANT 4, 7, 9: Xaa = Any Amino Acid
GPAXGLXGX

SEQ ID NO: 96 - protein, 8 aa, Artificial Sequence
  Synthetic Construct
GPLGIAGQ

SEQ ID NO: 97 - protein, 6 aa, Artificial Sequence
  Synthetic Construct
PVGLIG

SEQ ID NO: 98 - protein, 8 aa, Artificial Sequence
  Synthetic Construct
HPVGLLAR

SEQ ID NO: 99 - protein, 11 aa, Artificial Sequence
  Synthetic Construct
  VARIANT 1, 2, 3, 8, 9, 10, 11: Xaa = Any Amino Acid
XXXVIATXXXX

SEQ ID NO: 100 - protein, 10 aa, Artificial Sequence
  Synthetic Construct
  VARIANT 1, 7, 8, 9, 10: Xaa = Any Amino Acid
XYYVTAXXXX

SEQ ID NO: 101 - protein, 8 aa, Artificial Sequence
  Synthetic Construct
PRFKIIGG

SEQ ID NO: 102 - protein, 8 aa, Artificial Sequence
  Synthetic Construct
PRFRIIGG

SEQ ID NO: 103 - protein, 9 aa, Artificial Sequence
  Synthetic Construct
SSRHRRALD

SEQ ID NO: 104 - protein, 14 aa, Artificial Sequence
  Synthetic Construct
RKSSIIIRMRDVVL

SEQ ID NO: 105 - protein, 15 aa, Artificial Sequence
  Synthetic Construct
SSSFDKGKYKKGDDA

SEQ ID NO: 106 - protein, 15 aa, Artificial Sequence
  Synthetic Construct
SSSFDKGKYKRGDDA

SEQ ID NO: 107 - protein, 7 aa, Artificial Sequence
  Synthetic Construct
GGSIDGR

SEQ ID NO: 108 - protein, 6 aa, Artificial Sequence
  Synthetic Construct
PLGLWA

SEQ ID NO: 109 - protein, 8 aa, Artificial Sequence
  Synthetic Construct
DVAQFVLT

SEQ ID NO: 110 - protein, 448 aa, Artificial Sequence
  Synthetic Construct
EEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 111 - protein, 140 aa, Artificial Sequence
  Synthetic Construct
EEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPSHHHHHHGLNDIFEAQKIEWHE

SEQ ID NO: 112 - protein, 125 aa, Artificial Sequence
  Synthetic Construct
EEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPSHHHHHH

SEQ ID NO: 113 - protein, 118 aa, Artificial Sequence
  Synthetic Construct
DVQLVESGGGVVRPGESLRLSCAASGFTFSSYAMSWVRQAPGKGLEWLAGISAGGSDTYYIDSVKGRFTISRDNPKNSLYLQMSSLTAEDTAVYYCARETWNHLFDYWGLGTLVTVSS

SEQ ID NO: 114 - protein, 107 aa, Artificial Sequence
  Synthetic Construct
ALTQPASVSANPGETVKITCSGGDYYSTYYAWYQQKSPGSAPVTVIHSDDKRPSDIPSRFSGSASGSAATLIITGVRVEDEAVYYCGGYDGRTYINTFGAGTTLTVL

SEQ ID NO: 115 - protein, 118 aa, Artificial Sequence
  Synthetic Construct
DVQLVESGGGVVRPGESLRLSCAASGFTFSSNAMSWVRQAPGKGLEWLAGISAGGSDTYYPASVKGRFTISRDNSKNTLYLQMNTLTAEDTAVYYCARETWNHLFDYWGLGTLVTVSS

SEQ ID NO: 116 - protein, 107 aa, Artificial Sequence
  Synthetic Construct
ALTQPASVSANPGETVKIACSGGDYYSYYYGWYQQKAPGSALVTVIYSDDKRPSDIPSRFSGSASGSTATLTITGVRAEDEAVYYCGGYDYSTYANAFGAGTTLTVL

SEQ ID NO: 117 - protein, 441 aa, Artificial Sequence
  Synthetic Construct
DVQLVESGGGVVRPGESLRLSCAASGFTFSSNAMSWVRQAPGKGLEWLAGISAGGSDTYYPASVKGRFTISRDNSKNTLYLQMNTLTAEDTAVYYCARETWNHLFDYWGLGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFASTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFIYSKLNVQKSNWEAGNTFTCSVLHEGLHNHHTEKSLSHSPG

SEQ ID NO: 118 - protein, 213 aa, Artificial Sequence
  Synthetic Construct
ALTQPASVSANPGETVKIACSGGDYYSYYYGWYQQKAPGSALVTVIYSDDKRPSDIPSRFSGSASGSTATLTITGVRAEDEAVYYCGGYDYSTYANAFGAGTTLTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 119 - protein, 447 aa, Artificial Sequence
  Synthetic Construct
DVQLVESGGGVVRPGESLRLSCAASGFTFSSNAMSWVRQAPGKGLEWLAGISAGGSDTYYPASVKGRFTISRDNSKNTLYLQMNTLTAEDTAVYYCARETWNHLFDYWGLGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 120 - protein, 441 aa, Artificial Sequence
  Synthetic Construct
DVQLVESGGGVVRPGESLRLSCAASGFTFSSYAMSWVRQAPGKGLEWLAGISAGGSDTYYIDSVKGRFTISRDNPKNSLYLQMSSLTAEDTAVYYCARETWNHLFDYWGLGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFASTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFIYSKLNVQKSNWEAGNTFTCSVLHEGLHNHHTEKSLSHSPG

SEQ ID NO: 121 - protein, 213 aa, Artificial Sequence
  Synthetic Construct
ALTQPASVSANPGETVKITCSGGDYYSTYYAWYQQKSPGSAPVTVIHSDDKRPSDIPSRFSGSASGSAATLIITGVRVEDEAVYYCGGYDGRTYINTFGAGTTLTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 122 - protein, 447 aa, Artificial Sequence
  Synthetic Construct
DVQLVESGGGVVRPGESLRLSCAASGFTFSSYAMSWVRQAPGKGLEWLAGISAGGSDTYYIDSVKGRFTISRDNPKNSLYLQMSSLTAEDTAVYYCARETWNHLFDYWGLGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 123 - protein, 5 aa, Artificial Sequence
  Synthetic Construct
  VARIANT 5: Xaa = Any Amino Acid
GPGGX

SEQ ID NO: 124 - protein, 5 aa, Artificial Sequence
  Synthetic Construct
GPGQQ

SEQ ID NO: 125 - protein, 5 aa, Artificial Sequence
  Synthetic Construct
GPGGY

SEQ ID NO: 126 - protein, 25 aa, Artificial Sequence
  Synthetic Construct
GSGSHHHHHHGLNDIFEAQKIEWHE

SEQ ID NO: 127 - protein, 12 aa, Artificial Sequence
  Synthetic Construct
  VARIANT 4, 6, 7, 9: Xaa = Any Amino Acid
LSGXRXXSXDNH

SEQ ID NO: 128 - protein, 12 aa, Artificial Sequence
  Synthetic Construct
  VARIANT 1, 7, 10, 11, 12: Xaa = Any Amino Acid
XSGSRKXRVXXX

SEQ ID NO: 129 - protein, 6 aa, Artificial Sequence
  Synthetic Construct
  VARIANT 4: Xaa = Any Amino Acid
SGRXSA

SEQ ID NO: 130 - protein, 12 aa, Artificial Sequence
  Synthetic Construct
  VARIANT 4, 6, 7, 9: Xaa = Any Amino Acid
LSGXRXXSXDNH

SEQ ID NO: 131 - protein, 255 aa, Artificial Sequence
  Synthetic Construct
DVQLVESGGGVVRPGESLRLSCEASGFTFSSNAMSWVRQAPGKGLEWVAGISSGSDTYYGDSVKGRLTISRDNSKNILYLQMNSLTAEDTAVYYCARETWNHLFDYWGLGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPGSGSHHHHHHGLNDIFEAQKIEWHE

SEQ ID NO: 132 - protein, 214 aa, Artificial Sequence
  Synthetic Construct
ALTQPASVSASPGETVEITCSGGSDSSYYYGWYQQKSPGSAPVTVIYSDNKRPSNIPSRFSGSASGSTATLTITGVRVEDEAVYYCGGYDYSTYTNPFGAGTTLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - protein, 116 aa, Artificial Sequence
  Synthetic Construct
DVQLVESGGGVVRPGESLRLSCEASGFTFSSNAMSWVRQAPGKGLEWVAGISSGSDTYYGDSVKGRLTISRDNSKNILYLQMNSLTAEDTAVYYCARETWNHLFDYWGLGTLVTVS

SEQ ID NO: 134 - protein, 107 aa, Artificial Sequence
  Synthetic Construct
ALTQPASVSASPGETVEITCSGGSDSSYYYGWYQQKSPGSAPVTVIYSDNKRPSNIPSRFSGSASGSTATLTITGVRVEDEAVYYCGGYDYSTYTNPFGAGTTLTVL

SEQ ID NO: 135 - protein, 21 aa, Artificial Sequence
  Synthetic Construct
HHHHHHGLNDIFEAQKIEWHE

SEQ ID NO: 136 - protein, 12 aa, Artificial Sequence
  Synthetic Construct
GGSGGSGGSGGS

SEQ ID NO: 137 - protein, 12 aa, Artificial Sequence
  Synthetic Construct
  VARIANT 2, 3, 4, 7, 9, 10, 12: Xaa = Any Amino Acid
RXXXRKXVXXGX

SEQ ID NO: 138 - protein, 7 aa, Artificial Sequence
  Synthetic Construct
  VARIANT 5: Xaa = Any Amino Acid
RQARXVV

SEQ ID NO: 139 - protein, 5 aa, Artificial Sequence
  Synthetic Construct
  VARIANT 4: Xaa = Any Amino Acid
ATNXL

SEQ ID NO: 140 - protein, 4 aa, Artificial Sequence
  Synthetic Construct
IDGR

SEQ ID NO: 141 - protein, 4 aa, Artificial Sequence
  Synthetic Construct
IEGR

SEQ ID NO: 142 - protein, 4 aa, Artificial Sequence
  Synthetic Construct
VPGG

(19) United States
(12) Patent Application Publication
(10) Pub. No.: US 2022/0196651 A1
(43) Pub. Date: Jun. 23, 2022

(54) MULTIMERS FOR REDUCING THE INTERFERENCE OF DRUGS THAT BIND CD47 IN SEROLOGICAL ASSAYS

(71) Applicant: ALX Oncology Inc., South San Francisco, CA (US)

(72) Inventors: Jaume PONS, San Francisco, CA (US); Bang Janet SIM, South San Francisco, CA (US); Amy Shaw-Ru CHEN, San Jose, CA (US); Emma Ruth SANGALANG, Oakland, CA (US)

(21) Appl. No.: 17/543,569

(22) Filed: Dec. 6, 2021

Related U.S. Application Data
(60) Provisional application No. 63/121,964, filed on Dec. 6, 2020.

(51) Int. Cl. G01N 33/543; G01N 33/80; G01N 33/566; C07K 14/705; C07K 16/28

(52) U.S. Cl. CPC G01N 33/54393; G01N 33/80; G01N 33/566; C07K 2319/00; C07K 16/2803; G01N 2333/70596; G01N 2440/28; C07K 14/70503

(57) ABSTRACT

Provided are methods of reducing and/or preventing interference by a drug comprising (i) an antibody Fc region and (ii) a moiety that binds to human CD47 in serological assays.

### CROSS-REFERENCE TO RELATED APPLICATIONS

This application claims the priority benefit of U.S. Provisional Application No. 63/121,964, filed on Dec. 6, 2020, the contents of which are incorporated by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 757972001400SEQLIST.txt, date recorded: Dec. 3, 2021, size: 97,895 bytes).

### FIELD OF THE INVENTION

This invention relates to methods and reagents for use in reducing interference in serological assays by drugs that comprise (i) an antibody Fc region and (ii) a moiety that binds to human CD47.

### BACKGROUND OF THE INVENTION

An ever-increasing number of antibody-based drugs are being developed as treatments for a wide variety of diseases, including cancer. Such treatments have the potential of interfering with blood typing and serological assays if the target of the therapeutic antibody is also expressed on blood cells such as red blood cells (RBCs), white blood cells (WBC) and/or platelets.

For example, CD47, a widely-expressed cell surface protein that binds to signal regulatory protein-α (SIRPα ) and inhibits phagocytosis (Jaiswal et al., Trends Immunol (2010) 31(6):212-219; Brown et al., Trends Cell Biol (2001) 11(3):130-135), is expressed at high levels on a wide variety of malignant tumors, including hematological and solid tumors. Elevated CD47 expression also correlates with aggressive disease (Willingham et al., Proc Natl Acad Sci USA (2012) 109(17):6662-6667). Several cancer therapies targeting CD47, e.g., antibodies and fusion proteins comprising an antibody Fc region, have been developed to block the SIRPα-CD47 interaction, thereby permitting macrophages to carry out their phagocytic function to clear tumor cells.

As CD47 is also expressed on the surface of blood cells, such as red blood cells (RBCs) and platelets (Oldenborg et al., Science (2000) 288(5473):2051-2054), drugs comprising antibody Fc regions that target CD47 could interfere with blood typing and serological tests. Moreover, because patients receiving CD47-targeting drugs (e.g., for the treatment of cancer) often require blood transfusions to treat coincident anemia and/or thrombocytopenia, interference with serological and blood typing assays by anti-CD47 drugs is a significant patient safety concern. Thus, there is need in the art to develop methods and reagents to reduce interference of CD47-targeting drugs comprising antibody Fc regions with serological assays.

### SUMMARY OF THE INVENTION

In some embodiments, provided is a method of reducing drug interference in a serological assay using reagent red blood cells (RBC) or reagent platelets, said method comprising: (a) adding a CD47 multimer that binds to the drug and blocks the drug from binding the reagent RBC or the reagent platelets to a plasma sample from a subject who has received treatment with the drug; and (b) performing the serological assay of the plasma sample after step (a), using the reagent RBC or the reagent platelets, wherein the drug comprises (i) a human antibody Fc region or variant thereof and (ii) a moiety that binds to human CD47, and wherein the CD47 multimer comprises at least two CD47 polypeptide monomers.

In some embodiments, the CD47 multimer comprises between 2 and 100 CD47 polypeptide monomers. In some embodiments, the CD47 multimer comprises a CD47 polypeptide monomer that comprises a wild type CD47 or fragment thereof that is capable of binding the drug. In some embodiments, the CD47 multimer comprises a CD47 polypeptide monomer that comprises a wild type human CD47, a wild type mouse CD47, a wild type rat CD47, a wild type rhesus CD47, a wild type cynomolgus CD47, or a fragment of any one of the preceding that is capable of binding the drug. In some embodiments, the CD47 multimer comprises a CD47 polypeptide monomer that comprises the amino acid sequence of SEQ ID NO: 1. In some embodiments, the CD47 multimer comprises a CD47 polypeptide monomer that comprises a CD47 variant comprising one or more amino acid substitutions, insertions, deletions, N-terminal extensions, or C-terminal extensions relative to a wild type CD47 or fragment thereof that is capable of binding the drug. In some embodiments, the CD47 multimer comprises a CD47 polypeptide monomer that comprises the amino acid sequence set forth in any one of SEQ ID NOs: 2-6. In some embodiments, the CD47 multimer comprises a CD47 polypeptide monomer that comprises a fusion polypeptide. In some embodiments, the fusion polypeptide comprises a multimerization domain. In some embodiments, the multimerization domain comprises an Fc monomer, a c-Jun leucine zipper domain, or a c-Fos leucine zipper domain. In some embodiments, the Fc monomer is a murine Fc monomer. In some embodiments, the murine Fc monomer comprises an amino acid sequence set forth in any one of SEQ ID NO: 81-83. In some embodiments, the fusion polypeptide comprises an amino acid sequence set forth in any one of SEQ ID NO: 84-86. In some embodiments, the CD47 multimer comprises (e.g. further comprises) a CD47 polypeptide monomer that comprises an epitope tag or a ligand. In some embodiments, the epitope tag comprises any one of SEQ ID NOs: 7-32 and 126, or the ligand comprises biotin. In some embodiments, the CD47 multimer comprises a soluble CD47 polypeptide monomer.

In some embodiments, the CD47 multimer comprises at least two CD47 polypeptide monomers are linked via peptide bond. In some embodiments, the at least two CD47 polypeptide monomers are linked via linker peptide. In some embodiments, the linker peptide comprises any one of SEQ ID NO: 85-109, 127-130, 140, and 141. In some embodiments, the linker peptide comprises one or more spacers. In some embodiments, the spacer comprises GS, GGS, or any one of SEQ ID NOs: 52-70. In some embodiments, the CD47 multimer comprises at least two CD47 polypeptide monomers are attached to a solid support. In some embodiments, the solid support is a gold nanosphere, a gold nanoshell, a magnetic bead, a silica bead, a dextran polymer, a tube, a slide, a gel column, or a microtiter well. In some embodiments, each of the at least two CD47 polypeptide monomers comprises an epitope tag or a ligand, a capture agent that specifically binds the epitope tag or ligand is immobilized on the solid support, and the CD47 polypeptide monomers are attached to the solid support by the specific binding of the epitope tag or ligand by the capture agent. In some embodiments, the ligand is biotin and the capture agent is streptavidin. In some embodiments, at least one of the at least two CD47 polypeptide monomers comprises SEQ ID NO: 6. In some embodiments, the CD47 multimer comprises a streptavidin or avidin bound to 2, 3, or 4 biotinylated CD47 polypeptide monomers. In some embodiments, at least one of the 2, 3, or 4 biotinylated CD47 polypeptide monomers comprises SEQ ID NO: 6. In some embodiments, the CD47 multimer is a homomultimer. In some embodiments, the CD47 multimer is heteromultimer.

In some embodiments, provided is a method of reducing drug interference in a serological assay using reagent red blood cells (RBCs), reagent platelets, or a combination thereof said method comprising: (a) adding a SIRP multimer that specifically binds to human CD47 to the reagent red blood cells (RBCs), reagent platelets, or combination thereof; and (b) performing the serological assay of a plasma sample using the reagent red blood cells (RBCs), reagent platelets, or combination thereof of step (a), wherein the plasma sample is from a subject who has received treatment with a drug, wherein the drug comprises (i) an antibody Fc region and (ii) a moiety that binds to human CD47, and wherein the SIRP multimer comprises at least two SIRP polypeptide monomers. In some embodiments, provided is a method of reducing drug interference in a serological assay using reagent red blood cells (RBCs), reagent platelets, or a combination thereof, said method comprising: (a) adding a SIRP multimer that specifically binds to human CD47 to a plasma sample from a subject who has received treatment with a drug; and (b) performing the serological assay of the plasma sample after step (a) using the reagent red blood cells (RBCs), reagent platelets, or combination thereof, wherein the drug comprises (i) an antibody Fc region and (ii) a moiety that binds to human CD47, and wherein the SIRP multimer comprises at least two SIRP polypeptide monomers. In some embodiments, provided is a method of reducing drug interference in a serological assay of a blood sample containing reagent red blood cells (RBCs), reagent platelets, or a combination thereof, said method comprising: (a) adding a SIRP multimer that specifically binds to human CD47 to a blood sample from a subject who has received treatment with a drug; and (b) performing the serological assay of the blood sample after step (a), wherein the drug comprises (i) an antibody Fc region and (ii) a moiety that binds to human CD47, and wherein the SIRP multimer comprises at least two SIRP polypeptide monomers.

In some embodiments, the SIRP multimer comprises between 2 and 100 SIRP polypeptide monomers. In some embodiments, the SIRP multimer comprises a SIRP polypeptide monomer that comprises a wild type SIRPα or fragment thereof that is capable of binding human CD47. In some embodiments, the SIRP multimer comprises a SIRP polypeptide monomer that comprises a wild type human SIRPα, a wild type mouse SIRPα, a wild type rat SIRPα, a wild type rhesus SIRPα, a wild type cynomolgus SIRPα, or a fragment of any one of the preceding that is capable of binding human CD47. In some embodiments, the SIRP multimer comprises a SIRP polypeptide monomer that comprises a SIRPα variant comprising one or more amino acid substitutions, insertions, deletions, N-terminal extensions, or C-terminal extensions relative to a wild type SIRPα or a fragment thereof that is capable of binding to CD47. In some embodiments, the SIRP multimer comprises a SIRP polypeptide monomer that comprises a wild type SIRPγ or fragment thereof that is capable of binding human CD47. In some embodiments, the SIRP multimer comprises a SIRP polypeptide monomer that comprises a wild type human SIRPγ, a wild type mouse SIRPγ, a wild type rat SIRPγ, a wild type rhesus SIRPγ, a wild type cynomolgus SIRPγ, or a fragment of any one of the preceding that is capable of binding human CD47. In some embodiments, the SIRP multimer comprises a SIRP polypeptide monomer that comprises a SIRPγ variant comprising one or more amino acid substitutions, insertions, deletions, N-terminal extensions, or C-terminal extensions relative to a wild type SIRPγ or fragment thereof that is capable of binding to CD47. In some embodiments, the SIRP multimer comprises a SIRP polypeptide monomer that comprises a SIRPβ variant comprising one or more amino acid substitutions, insertions, deletions, N-terminal extensions, or C-terminal extensions relative to a wild type SIRPβ or fragment thereof, the SIRPβ variant or fragment thereof is capable of binding to CD47. In some embodiments, SIRP multimer comprises a SIRP polypeptide monomer that comprises the amino acid sequence of any one of SEQ ID NOs: 33-45. In some embodiments, the SIRP multimer comprises a SIRP polypeptide monomer that comprises a fusion polypeptide. In some embodiments, the fusion polypeptide comprises a multimerization domain. In some embodiments, the multimerization domain comprises an Fc monomer, a c-Jun leucine zipper domain, or a c-Fos leucine zipper domain. In some embodiments, the Fc monomer is a murine Fc monomer. In some embodiments, the murine Fc monomer comprises an amino acid sequence set forth in any one of SEQ ID NO: 81-83. In some embodiments, the fusion polypeptide comprises an amino acid sequence set forth in SEQ ID NO: 110. In some embodiments, the SIRP multimer comprises a SIRP polypeptide monomer that comprises an epitope tag or a ligand. In some embodiments, the epitope tag comprises any one of SEQ ID NOs: 7-32 and 126, or the ligand comprises biotin. In some embodiments, the SIRP multimer comprises a soluble SIRP polypeptide monomer.

In some embodiments, the SIRP multimer comprises least two SIRP polypeptide monomers are linked via peptide bond. In some embodiments, the SIRP multimer comprises at least two SIRP polypeptide monomers are linked via linker peptide. In some embodiments, the linker peptide comprises any one of SEQ ID NO: 85-109, 127-130, 140, and 141. In some embodiments, the linker peptide comprises one or more spacers. In some embodiments, the spacer comprises GS, GGS, or any one of SEQ ID NOs: 52-70. In some embodiments, the SIRP multimer comprises at least two SIRP polypeptide monomers are attached to a solid support. In some embodiments, the solid support is a gold nanosphere, a gold nanoshell, a magnetic bead, a silica bead, a dextran polymer, a tube, a slide, a gel column, or a microtiter well. In some embodiments, each of the at least two SIRP polypeptide monomers comprises an epitope tag or a ligand, a capture agent that specifically binds the epitope tag or ligand is immobilized on the solid support, and the SIRP polypeptide monomer are attached to the solid support by the specific binding of the epitope tag or ligand by the capture agent. In some embodiments, the ligand is biotin and the capture agent is streptavidin. In some embodiments, at least one of the at least two SIRP polypeptide monomers comprises SEQ ID NO: 111. In some embodiments, the SIRP multimer comprises a streptavidin or avidin bound to 2, 3, or 4 biotinylated SIRP polypeptide monomers. In some embodiments, at least one of the 2, 3, or 4 biotinylated SIRP polypeptide monomers comprise SEQ ID NO: 111. In some embodiments, the SIRP multimer is a homomultimer. In some embodiments, the SIRP multimer is heteromultimer.

In some embodiments, provided is a method of reducing drug interference in a serological assay using reagent red blood cells (RBC) or reagent platelets, said method comprising: (a) adding an anti-SIRP multimer that binds to the drug and blocks the drug from binding the reagent RBC or the reagent platelets to a plasma sample from a subject who has received treatment with the drug; and (b) performing the serological assay of the plasma sample after step (a), using the reagent RBC or the reagent platelets, wherein the drug comprises (i) a human antibody Fc region or variant thereof and (ii) a moiety that binds to human CD47, and wherein the anti-SIRP multimer comprises one or more anti-SIRP antibodies or drug-binding fragments thereof.

In some embodiments, the anti-SIRP multimer comprises an anti-SIRP antibody or drug-binding fragment thereof. In some embodiments, the anti-SIRP multimer comprises between 1 and 100 anti-SIRP antibodies or drug-binding fragments thereof. In some embodiments, the anti-SIRP multimer comprises an anti-SIRP antibody or drug-binding fragment thereof that binds to a wild type SIRPα, a SIRPα variant, a SIRPβ variant, a wild-type SIRPγ, a SIRPγ variant, or any two or more of the preceding. In some embodiments, the anti-SIRP multimer comprises an anti-SIRP antibody or drug-binding fragment thereof that comprises: (a) a heavy chain variable domain (V_{H}) that comprises SEQ ID NO: 46 and a light chain variable domain (V_{L}) that comprises SEQ ID NO: 47; (b) a heavy chain variable domain (V_{H}) that comprises SEQ ID NO: 48 and a light chain variable domain (V_{L}) that comprises SEQ ID NO: 49; (c) a heavy chain variable domain (V_{H}) that comprises SEQ ID NO: 50 and a light chain variable domain (V_{L}) that comprises SEQ ID NO: 51; (d) a heavy chain variable domain (V_{H}) that comprises SEQ ID NO: 113 and a light chain variable domain (V_{L}) that comprises SEQ ID NO: 114; (e) a heavy chain variable domain (V_{H}) that comprises SEQ ID NO: 115 and a light chain variable domain (V_{L}) that comprises SEQ ID NO: 116; and/or (f) a heavy chain variable domain (V_{H}) that comprises SEQ ID NO: 133 and a light chain variable domain (V_{L}) that comprises SEQ ID NO: 134. In some embodiments, the anti-SIRP multimer comprises a full length anti-SIRP antibody. In some embodiments, the anti-SIRP antibody comprises a murine Fc domain. In some embodiments, the murine Fc domain comprises an amino acid sequence set forth in any one of SEQ ID NOs: 81-83. In some embodiments, the anti-SIRP antibody comprises: (a) a heavy chain that comprises SEQ ID NO: 117 and a light chain that comprises SEQ ID NO: 118; (b) a heavy chain that comprises SEQ ID NO: 119 and a light chain that comprises SEQ ID NO: 118; (c) a heavy chain that comprises SEQ ID NO: 120 and a light chain that comprises SEQ ID NO: 121; or (d) a heavy chain that comprises SEQ ID NO: 122 and a light chain that comprises SEQ ID NO: 121. In some embodiments, the drug-binding fragment of the anti-SIRP antibody is a Fab, a Fab′, an F(ab′)2, a Fab′-SH, an Fv, a diabody, a one-armed antibody, an scFv, an scFv-Fc, a single domain antibody, or a single heavy chain antibody. In some embodiments, the drug binding fragment comprises a F(ab′)2, and the F(ab′)2 comprises SEQ ID NOs: 131 and 132. In some embodiments, the anti-SIRP antibody or drug-binding fragment thereof comprises an epitope tag or a ligand. In some embodiments, epitope tag comprises any one of SEQ ID NOs: 7-32 and 126, or the ligand comprises biotin. In some embodiments, the epitope tag comprises HHHHHHGLNDIFEAQKIEWHE (SEQ ID NO: 135) or GSGSHHHHHHGLNDIFEAQKIEWHE (SEQ ID NO: 126).

In some embodiments, the anti-SIRP multimer comprises one or more anti-SIRP antibodies or drug-binding fragments thereof attached to a solid support. In some embodiments, the solid support is a gold nanosphere, a gold nanoshell, a magnetic bead, a silica bead, a dextran polymer, a tube, a slide, a gel column, or a microtiter well. In some embodiments, the one or more anti-SIRP antibodies or drug-binding fragments thereof comprises an epitope tag or a ligand, a capture agent that specifically binds the epitope tag or ligand is immobilized on the solid support, and the anti-SIRP antibodies or drug-binding fragments thereof are attached to the solid support by the specific binding of the epitope tag or ligand by the capture agent. In some embodiments, the ligand is biotin and the capture agent is streptavidin. In some embodiments, the anti-SIRP multimer comprises a streptavidin or avidin bound to 2, 3, or 4 biotinylated anti-SIRP antibodies or fragments thereof. In some embodiments, the anti-SIRP multimer comprises the streptavidin or the avidin bound to 2, 3, or 4 biotinylated F(ab′)2 fragments, 2 or more of the biotinylated F(ab′)2 fragments comprise SEQ ID NOs: 131 and 132. In some embodiments, the anti-SIRP multimer is a homomultimer. In some embodiments, the anti-SIRP multimer is heteromultimer.

In some embodiments of any of the methods herein, the drug comprises an anti-CD47 antibody. In some embodiments, the moiety of the drug that binds to human CD47 comprises a wild type SIRPα, a SIRPα variant, or a fragment of the wild type SIRPα or the SIRPα variant. In some embodiments, the moiety of the drug that binds to human CD47 comprises the SIRPα variant, and the SIRPα variant comprises one or more amino acid substitution(s), insertion(s), deletion(s), N-terminal extension(s), and/or C-terminal extension(s) relative to the wild type SIRPα. In some embodiments, the moiety of the drug that binds to human CD47 comprises the fragment of the SIRPα variant, and the fragment comprises an extracellular domain of the SIRPα variant. In some embodiments, the moiety of the drug that binds to human CD47 comprises a wild type SIRPγ, a SIRPγ variant, or a fragment of the wild type SIRPγ or the SIRPγ variant. In some embodiments, the moiety of the drug that binds to human CD47 comprises the SIRPγ variant, and the SIRPγ variant comprises one or more amino acid substitution(s), insertion(s), deletion(s), N-terminal extension(s), C-terminal extension(s), or any combination of the preceding, relative to the wild type SIRPγ. In some embodiments, the moiety of the drug that binds to human CD47 comprises the fragment of the SIRPγ variant, and the fragment comprises an extracellular domain of the SIRPγ variant. In some embodiments, the moiety of the drug that binds to human CD47 comprises a SIRPβ variant or a fragment of the SIRPβ variant. In some embodiments, the moiety of the drug that binds to human CD47 comprises the SIRPβ variant, and the SIRPβ variant comprises one or more amino acid substitution(s), insertion(s), deletion(s), N-terminal extension(s), C-terminal extension(s), or any combination of the preceding, relative to the wild type SIRPβ. In some embodiments, the moiety of the drug that binds to human CD47 comprises the fragment of the SIRPβ variant, and the fragment comprises an extracellular domain of the SIRPβ variant. In some embodiments, the antibody Fc region of the drug is a human IgG Fc region or a variant thereof. In some embodiments, the human IgG Fc region is an IgG1, IgG2, or IgG4 Fc region, or a variant of an IgG1, IgG2, or IgG4 Fc region.

In some embodiments of any of the methods herein, the serological assay is an ABO/Rh typing assay. In some embodiments, the serological assay is an immediate spin (IS) assay. In some embodiments, the serological assay is a direct antiglobulin (DAT) assay using a polyspecific reagent that detects IgG and complement C3. In some embodiments, the serological assay is a direct antiglobulin (DAT) assay using a monospecific reagent that detects complement C3. In some embodiments, the serological assay is a PEG-enhanced serological assay. In some embodiments, the serological assay is an eluate test that is performed following the DAT assay. In some embodiments, the serological assay is a tube assay or a solid phase red cell assay (SPRCA).

Provided herein is a CD47 multimer comprising at least two CD47 polypeptide monomers. In some embodiments, the CD47 multimer comprises between 2 and 100 CD47 polypeptide monomers. In some embodiments, the CD47 multimer comprises a CD47 polypeptide monomer that comprises a wild type CD47 or fragment thereof that is capable of binding the drug. In some embodiments, the CD47 multimer comprises a CD47 polypeptide monomer that comprises a wild type human CD47, a wild type mouse CD47, a wild type rat CD47, a wild type rhesus CD47, a wild type cynomolgus CD47, or a fragment of any one of the preceding that is capable of binding the drug. In some embodiments, the CD47 multimer comprises a CD47 polypeptide monomer that comprises the amino acid sequence of SEQ ID NO: 1. In some embodiments, the CD47 multimer comprises a CD47 polypeptide monomer that comprises a CD47 variant comprising one or more amino acid substitutions, insertions, deletions, N-terminal extensions, or C-terminal extensions relative to a wild type CD47 or fragment thereof that is capable of binding the drug. In some embodiments, the CD47 multimer comprises a CD47 polypeptide monomer that comprises the amino acid sequence set forth in any one of SEQ ID NOs: 2-6. In some embodiments, the CD47 multimer comprises a CD47 polypeptide monomer that comprises a fusion polypeptide. In some embodiments, the fusion polypeptide comprises a multimerization domain. In some embodiments, the multimerization domain comprises an Fc monomer, a c-Jun leucine zipper domain, or a c-Fos leucine zipper domain. In some embodiments, the Fc monomer is a murine Fc monomer. In some embodiments, the murine Fc monomer comprises an amino acid sequence set forth in any one of SEQ ID NO: 81-83. In some embodiments, the fusion polypeptide CD47 polypeptide monomer comprises an amino acid sequence set forth in any one of SEQ ID NO: 84-86. In some embodiments, the CD47 multimer comprises a CD47 polypeptide monomer that comprises an epitope tag or a ligand. In some embodiments, the epitope tag comprises any one of SEQ ID NOs: 7-32 and 126, or the ligand comprises biotin. In some embodiments, the CD47 multimer comprises a soluble CD47 polypeptide monomer.

In some embodiments, the CD47 multimer comprises at least two CD47 polypeptide monomers linked via peptide bond. In some embodiments, the CD47 multimer comprises at least two CD47 polypeptide monomers linked via linker peptide. In some embodiments, the linker peptide comprises any one of SEQ ID NO: 85-109, 127-130, 140, and 141. In some embodiments, the linker peptide comprises one or more spacers. In some embodiments, the spacer comprises GS, GGS, or any one of SEQ ID NOs: 52-70. In some embodiments, the CD47 multimer comprises at least two CD47 multimer comprises more than one CD47 polypeptide monomers are attached to a solid support. In some embodiments, the solid support is a gold nanosphere, a gold nanoshell, a magnetic bead, a silica bead, a dextran polymer, a tube, a slide, a gel column, or a microtiter well. In some embodiments, each of the at least two CD47 polypeptide monomers comprises an epitope tag or a ligand, a capture agent that specifically binds the epitope tag or ligand is immobilized on the solid support, and the CD47 polypeptide monomers are attached to the solid support by the specific binding of the epitope tag or ligand by the capture agent. In some embodiments, the ligand is biotin and the capture agent is streptavidin. In some embodiments, at least one of the at least two CD47 polypeptide monomers comprises SEQ ID NO: 6. In some embodiments, the CD47 multimer comprises a streptavidin or avidin bound to 2, 3, or 4 biotinylated CD47 polypeptide monomers. In some embodiments, at least one of the 2, 3, or 4 biotinylated CD47 polypeptide monomers comprises SEQ ID NO: 6. In some embodiments, the CD47 multimer is a homomultimer. In some embodiments, the CD47 multimer is a heteromultimer.

In some embodiments, provided is a SIRP multimer comprising at least two SIRP polypeptide monomers. In some embodiments, the SIRP multimer comprises between 2 and 100 SIRP polypeptide monomers. In some embodiments, the SIRP multimer comprises a SIRP polypeptide monomer that comprises a wild type SIRPα or fragment thereof that is capable of binding human CD47. In some embodiments, the SIRP multimer comprises the SIRP multimer comprises a SIRP polypeptide monomer that comprises a wild type human SIRPα, a wild type mouse SIRPα, a wild type rat SIRPα, a wild type rhesus SIRPα, a wild type cynomolgus SIRPα, or a fragment of any one of the preceding that is capable of binding human CD47. In some embodiments, the SIRP multimer comprises a SIRP polypeptide monomer that comprises a SIRPα variant comprising one or more amino acid substitutions, insertions, deletions, N-terminal extensions, or C-terminal extensions relative to a wild type SIRPα or a fragment thereof that is capable of binding to CD47. In some embodiments, the SIRP multimer comprises a SIRP polypeptide monomer that comprises a wild type SIRPγ or fragment thereof that is capable of binding human CD47. In some embodiments, the SIRP multimer comprises a SIRP polypeptide monomer that comprises a wild type human SIRPγ, a wild type mouse SIRPγ, a wild type rat SIRPγ, a wild type rhesus SIRPγ, a wild type cynomolgus SIRPγ, or a fragment of any one of the preceding that is capable of binding human CD47. In some embodiments, the SIRP multimer comprises a SIRP polypeptide monomer that comprises a SIRPγ variant comprising one or more amino acid substitutions, insertions, deletions, N-terminal extensions, or C-terminal extensions relative to a wild type SIRPγ or fragment thereof that is capable of binding to CD47. In some embodiments, the SIRP multimer comprises a SIRP polypeptide monomer that comprises a SIRPβ variant comprising one or more amino acid substitutions, insertions, deletions, N-terminal extensions, or C-terminal extensions relative to a wild type SIRPβ or fragment thereof, the SIRPβ variant or fragment thereof is capable of binding to CD47. In some embodiments, the SIRP multimer comprises a SIRP polypeptide monomer that comprises the amino acid sequence of any one of SEQ ID NOs: 33-45. In some embodiments, the SIRP multimer comprises a SIRP polypeptide monomer that comprises a fusion polypeptide. In some embodiments, the fusion polypeptide comprises a multimerization domain. In some embodiments, the multimerization domain comprises an Fc monomer, a c-Jun leucine zipper domain, or a c-Fos leucine zipper domain. In some embodiments, the Fc monomer is a murine Fc monomer. In some embodiments, the murine Fc monomer comprises an amino acid sequence set forth in any one of SEQ ID NO: 81-83. In some embodiments, the fusion polypeptide comprises an amino acid sequence set forth in SEQ ID NO: 110. In some embodiments, the SIRP multimer comprises a SIRP polypeptide monomer that comprises an epitope tag or a ligand. In some embodiments, the epitope tag comprises any one of SEQ ID NOs: 7-32 and 126, or the ligand comprises biotin. In some embodiments, the SIRP multimer comprises a soluble SIRP polypeptide monomer.

In some embodiments, the SIRP multimer comprises at least two SIRP polypeptide monomers linked via peptide bond. In some embodiments, the SIRP multimer comprises at least two SIRP polypeptide monomers linked via linker peptide. In some embodiments, the linker peptide comprises any one of SEQ ID NO: 85-109, 127-130, 140, and 141. In some embodiments, the linker peptide comprises one or more spacers. In some embodiments, the spacer comprises GS, GGS, or any one of SEQ ID NOs: 52-70. In some embodiments, the SIRP multimer comprises at least two SIRP polypeptide monomers are attached to a solid support. In some embodiments, the solid support is a gold nanosphere, a gold nanoshell, a magnetic bead, a silica bead, a dextran polymer, a tube, a slide, a gel column, or a microtiter well. In some embodiments, each of the at least two SIRP polypeptide monomers comprises an epitope tag or a ligand, a capture agent that specifically binds the epitope tag or ligand is immobilized on the solid support, and the SIRP polypeptide monomer are attached to the solid support by the specific binding of the epitope tag or ligand by the capture agent. In some embodiments, the ligand is biotin and the capture agent is streptavidin. In some embodiments, at least one of the at least two SIRP polypeptide monomers comprises SEQ ID NO: 111. In some embodiments, the SIRP multimer comprises a streptavidin or avidin bound to 2, 3, or 4 biotinylated SIRP polypeptide monomers. In some embodiments, at least one of the 2, 3, or 4 biotinylated SIRP polypeptide monomers comprise SEQ ID NO: 111. In some embodiments, the SIRP multimer is a homomultimer. In some embodiments, the SIRP multimer is heteromultimer.

In some embodiments, provided herein is an anti-SIRP multimer comprising one or more anti-SIRP antibodies or drug-binding fragments thereof. In some embodiments, the anti-SIRP multimer comprises between 1 and 100 anti-SIRP antibodies or drug-binding fragments thereof. In some embodiments, the anti-SIRP multimer comprises an anti-SIRP antibody or drug-binding fragment thereof that binds to a wild type SIRPα, a SIRPα variant, a SIRPβ variant, a wild-type SIRPγ, a SIRPγ variant, or any two or more of the preceding. In some embodiments, the anti-SIRP multimer comprises an anti-SIRP antibody or drug-binding fragment thereof that comprises: (a) a heavy chain variable domain (V_{H}) that comprises SEQ ID NO: 46 and a light chain variable domain (V_{L}) that comprises SEQ ID NO: 47; (b) a heavy chain variable domain (V_{H}) that comprises SEQ ID NO: 48 and a light chain variable domain (V_{L}) that comprises SEQ ID NO: 49; (c) a heavy chain variable domain (V_{H}) that comprises SEQ ID NO: 50 and a light chain variable domain (V_{L}) that comprises SEQ ID NO: 51; (d) a heavy chain variable domain (V_{H}) that comprises SEQ ID NO: 113 and a light chain variable domain (V_{L}) that comprises SEQ ID NO: 114; (e) a heavy chain variable domain (V_{H}) that comprises SEQ ID NO: 115 and a light chain variable domain (V_{L}) that comprises SEQ ID NO: 116; and/or (f) a heavy chain variable domain (V_{H}) that comprises SEQ ID NO: 133 and a light chain variable domain (V_{L}) that comprises SEQ ID NO: 134. In some embodiments, the anti-SIRP multimer comprises a full length anti-SIRP antibody. In some embodiments, the anti-SIRP antibody comprises a murine Fc domain. In some embodiments, the murine Fc domain comprises an amino acid sequence set forth in any one of SEQ ID Nos: 81-83. In some embodiments, the anti-SIRP antibody comprises: (a) a heavy chain that comprises SEQ ID NO: 117 and a light chain that comprises SEQ ID NO: 118; (b) a heavy chain that comprises SEQ ID NO: 119 and a light chain that comprises SEQ ID NO: 118; (c) a heavy chain that comprises SEQ ID NO: 120 and a light chain that comprises SEQ ID NO: 121; or (d) a heavy chain that comprises SEQ ID NO: 122 and a light chain that comprises SEQ ID NO: 121. In some embodiments, the drug-binding fragment of the anti-SIRP antibody is a Fab, a Fab′, an F(ab′)2, a Fab′-SH, an Fv, a diabody, a one-armed antibody, an scFv, an scFv-Fc, a single domain antibody, or a single heavy chain antibody. In some embodiments, the drug binding fragment comprises a F(ab′)2, and the F(ab′)2 comprises SEQ ID NOs: 131 and 132. In some embodiments, the anti-SIRP antibody or drug-binding fragment thereof comprises an epitope tag or a ligand. In some embodiments, the epitope tag comprises any one of SEQ ID NOs: 7-32 and 126, or the ligand comprises biotin. In some embodiments, the epitope tag comprises HHHHHHGLNDIFEAQKIEWHE (SEQ ID NO: 135) or GSGSHHHHHHGLNDIFEAQKIEWHE (SEQ ID NO: 126).

In some embodiments, the anti-SIRP multimer comprises the one or more anti-SIRP antibodies or drug-binding fragments thereof are attached to a solid support. In some embodiments, the solid support is a gold nanosphere, a gold nanoshell, a magnetic bead, a silica bead, a dextran polymer, a tube, a slide, a gel column, or a microtiter well. In some embodiments, each of the one or more anti-SIRP antibodies or drug-binding fragments thereof comprises an epitope tag or a ligand, a capture agent that specifically binds the epitope tag or ligand is immobilized on the solid support, and the anti-SIRP antibodies or drug-binding fragments thereof are attached to the solid support by the specific binding of the epitope tag or ligand by the capture agent. In some embodiments, the ligand is biotin and the capture agent is streptavidin. In some embodiments, the anti-SIRP multimer comprises a streptavidin or avidin bound to 2, 3, or 4 biotinylated anti-SIRP antibodies or fragments thereof. In some embodiments, the anti-SIRP multimer comprises the streptavidin or the avidin bound to 2, 3, or 4 biotinylated F(ab′)2 fragments, wherein 2 or more of the biotinylated F(ab′)2 fragments comprise SEQ ID NOs: 131 and 132. In some embodiments, the anti-SIRP multimer is a homomultimer. In some embodiments, the anti-SIRP multimer is heteromultimer.

All references cited herein, including patent applications, patent publications, and UniProtKB/Swiss-Prot Accession numbers are herein incorporated by reference in their entirety, as if each individual reference were specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows a serological assay in which a plasma sample obtained from a subject is mixed with reagent red blood cells (i.e., red blood cells (“RBCs”) that are known to express a particular cell surface antigen, or group of cell surface antigens) to detect the presence of antibodies in the plasma sample that bind the RBC surface antigen. Alternatively, such serological assay can be performed using reagent platelets (i.e., platelets that are known to express a particular cell surface antigen, or group of cell surface antigens) instead of reagent RBCs.

FIG. 1B shows how the presence of a drug comprising (i) an antibody Fc region and (ii) a moiety that binds human CD47 in a plasma sample interferes with the assay of FIG. 1A.

FIG. 1C shows a serological assay in which a blood sample obtained from a subject is mixed with reagent plasma/antisera (i.e., plasma or antisera known to contain antibodies against a specific RBC surface antigen(s) or platelet surface antigen(s)) in order to detect the presence of the antigen on the subject's RBCs and/or platelets.

FIG. 1D shows how the presence of a drug comprising (i) an antibody Fc region and (ii) a moiety that binds human CD47 in a blood sample interferes with the assay of FIG. 1C.

FIG. 2 shows a method of reducing interference in a serological assay that comprises adding a CD47 multimer to a plasma sample obtained from a subject who has been treated with the drug. Briefly, the CD47 multimer binds the drug in the plasma sample so that little or no free drug is available to bind the CD47 on the surface of the reagent RBCs or reagent platelets.

FIG. 3A shows a method of reducing interference in a serological assay that comprises adding a SIRP multimer that binds CD47 to reagent RBCs or reagent platelets. Briefly, the SIRP multimer binds to the CD47 on the surface of the reagent RBCs or reagent platelets. Binding of the reagent RBCs (or reagent platelets) by the SIRP multimer blocks the drug from binding the reagent RBCs (or reagent platelets).

FIG. 3B shows a method of reducing interference in a serological assay that comprises adding a SIRP multimer that binds CD47 to plasma from a subject who has received treatment with the drug. Briefly, the SIRP multimer competes with the drug for binding to the CD47 expressed on the surface of the reagent RBCs or reagent platelets and minimizes the amount of drug-bound reagent RBCs or drug-bound reagent platelets in the assay (or eliminates drug-bound reagent RBC and/or drug-bound reagent platelets in the assay).

FIG. 3C shows a method of reducing interference in a serological assay that comprises adding a SIRP multimer that binds CD47 to a blood sample from a subject who has received treatment with the drug. Briefly, the SIRP multimer competes with the drug for binding to the CD47 expressed on the surface of the subject's RBCs and/or platelets and minimizes (or eliminates) the amount of drug-bound RBC and/or drug-bound platelets in the assay.

FIG. 3D shows a method of reducing interference in a serological assay that comprises adding a SIRP multimer that binds drug to a plasma sample obtained from a subject who has been treated with the drug. Briefly, the SIRP multimer binds the drug in the plasma sample so that little or no free drug is available to bind the CD47 on the surface of the reagent RBCs or reagent platelets.

FIG. 4 provides the results of experiments that were performed to compare the degree to which CD47 polypeptide monomer, CD47 multimer B, anti-SIRP multimer C, and anti-SIRP multimer D inhibit interference by Drug A in an IAT tube assay.

FIG. 5 provides the results of further experiments that were performed to compare the degree to which CD47 polypeptide monomer, CD47 multimer B, anti-SIRP multimer C, and anti-SIRP multimer D inhibit interference by Drug A in an IAT tube assay.

FIG. 6 provides the results of further experiments that were performed to compare the degree to which CD47 polypeptide monomer, CD47 multimer B, anti-SIRP multimer C, and anti-SIRP multimer D inhibit interference by Drug A in an IAT tube assay.

FIG. 7 provides the results of experiments that were performed to compare the degree to which CD47 polypeptide monomer, CD47 multimer B, anti-SIRP multimer C, and anti-SIRP multimer D inhibit interference by Drug A in a solid phase red cell adherence assay (SPRCA).

FIG. 8 provides the results of experiments that were performed to test whether anti-SIRP multimer E or anti-SIRP multimer F inhibit interference by Drug A in a solid phase red cell adherence assay (SPRCA).

### DETAILED DESCRIPTION OF THE INVENTION

### I. Methods of Mitigating Interference in Pre-Transfusion Serological Assays

CD47 is a transmembrane protein that interacts with thrombospondin-1 (TSP-1) as well as several molecules on immune cells, including signal regulatory protein alpha (SIRPα). Upon binding CD47, SIRPα initiates a signaling cascade that inhibits phagocytosis and prevents phagocytic removal of healthy cells by the immune system. However, many cancers overexpress CD47 and evade phagocytic clearance. Accordingly, drugs that target CD47 (such as anti-CD47 antibodies and fusion proteins comprising an antibody Fc region and a moiety that binds CD47) are of significant therapeutic interest. CD47 is also expressed on the surface of human red blood cells (RBCs) and platelets. Thus, following the administration of a drug comprising (i) an antibody Fc region and (ii) a moiety that binds to human CD47 to a subject, the drug present in the subject's plasma or bound to the subject's RBCs and/or platelets may cause interference in routine pre-transfusion serological assays.

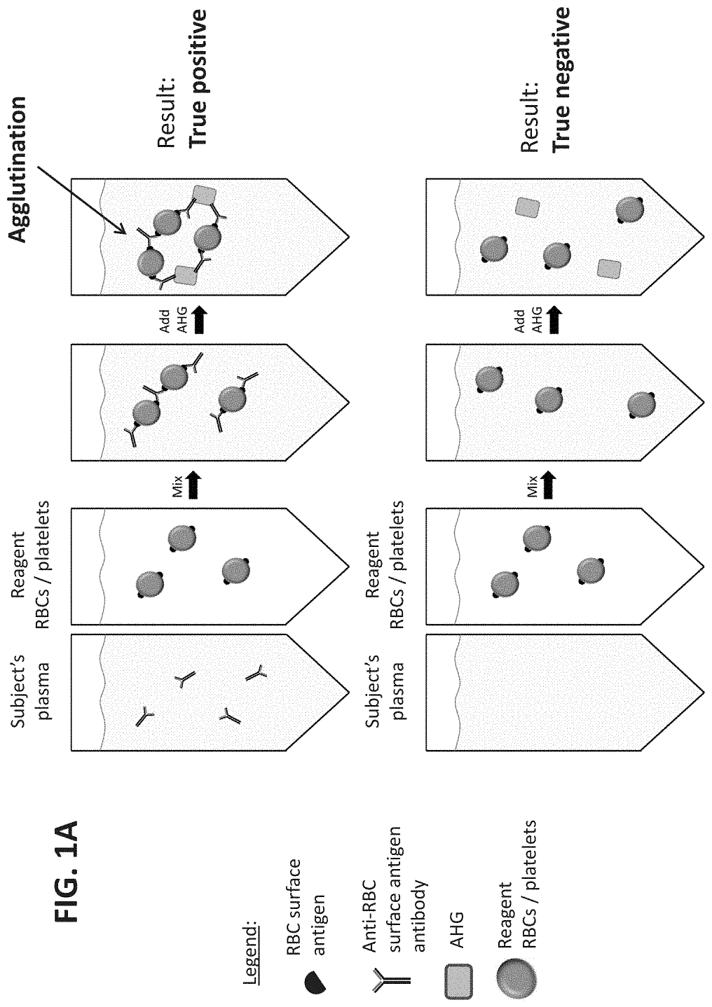

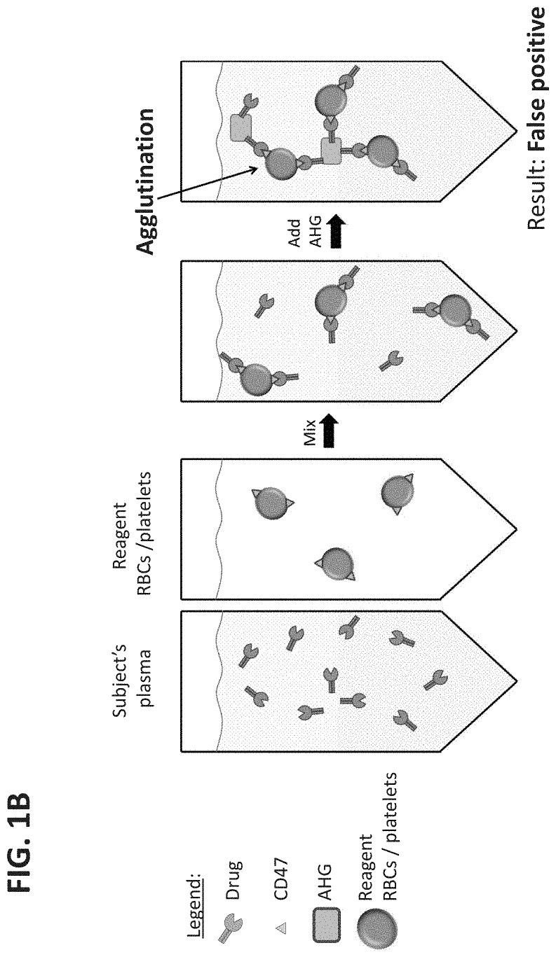

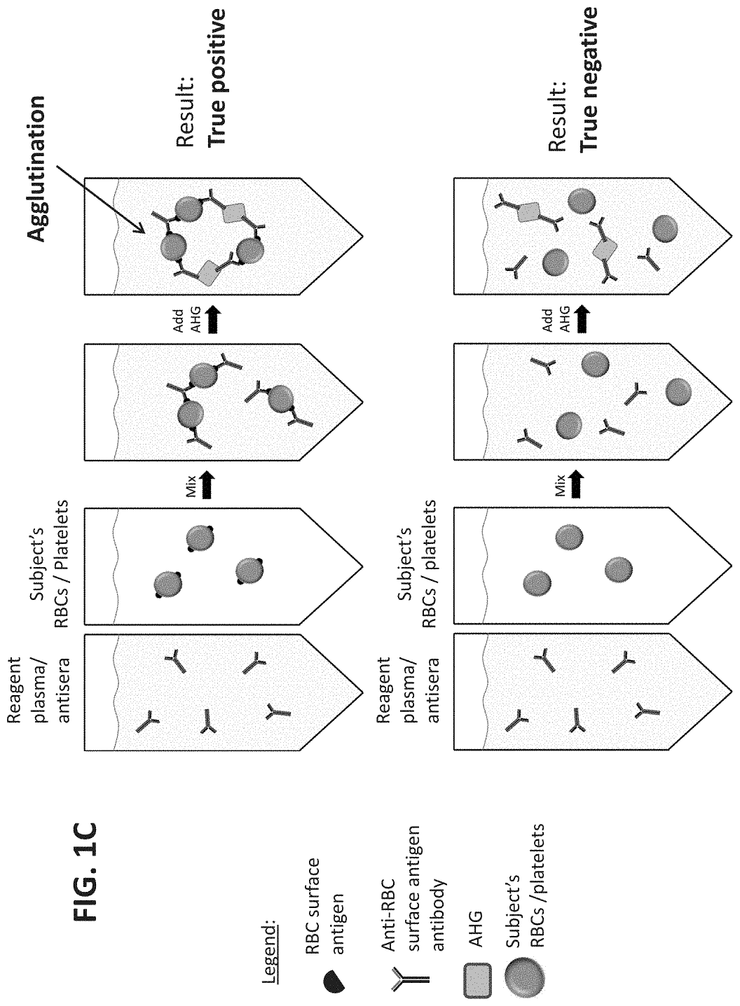

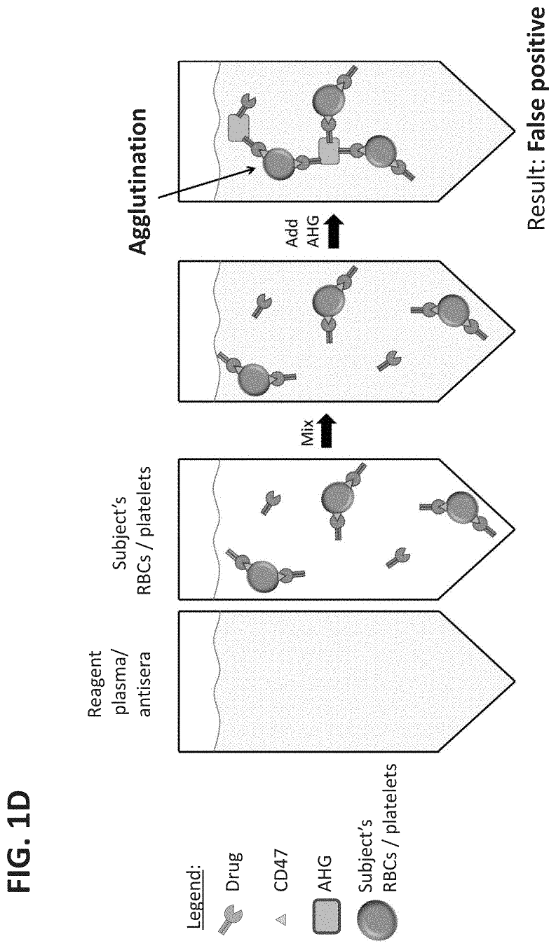

For example, FIG. 1A shows a serological assay in which a plasma sample obtained from a subject is mixed with reagent RBC or “reference RBC” (i.e., RBC that are known to express a particular cell surface antigen, or group of cell surface antigens) or reagent platelets or “reference platelets” (i.e., platelets that are known to express a particular cell surface antigen, or group of cell surface antigens) to detect the presence of antibodies in the plasma sample that bind the cell surface antigen that is known to be expressed on the reagent RBCs or reagent platelets. After the plasma sample and the reagent RBC (or reagent platelets) are mixed, anti-human globulin (AHG) is added, and agglutination (e.g., clumping) of the reagent RBC (or reagent platelets) occurs if the plasma sample contains an antibody that binds the RBC surface antigen (or platelet surface antigen). However, the presence of a drug comprising (i) an antibody Fc region and (ii) a moiety that binds to human CD47 in the subject's plasma may interfere with the assay and produce a false positive result. As shown in FIG. 1B, after the subject's plasma and the reagent RBCs (or reagent platelets) are mixed, the drug may bind CD47 that is expressed on the surface of reagent RBCs (or reagent platelets). Addition of AHG to the mix leads to agglutination of the reagent RBCs (or reagent platelets).

FIG. 1C depicts a serological assay in which a blood sample from a subject is mixed with reagent plasma/antisera (i.e., plasma or antisera containing antibodies against a known RBC surface antigen(s) or a known platelet surface antigen(s)) in order to detect the presence of the antigen on the subject's RBCs and/or platelets. After the reagent plasma/antisera and the sample from subject are mixed, the addition of AHG will lead to agglutination if the antigen recognized by the antibodies in the reagent plasma/antisera is expressed on the subject's RBCs and/or platelets. The presence of a drug comprising (i) an antibody Fc region and (ii) a moiety that binds to human CD47 in a sample comprising the subject's RBCs and/or platelets may interfere with the assay and produce a false positive result. As shown in FIG. 1D, the drug bound to the CD47 on the subject's RBCs or platelets will cause agglutination after AHG is added to a mixture comprising the subject's blood sample and reagent plasma/antisera.

The methods described below reduce (and, in some embodiments, eliminate) the interference caused by the drug, i.e., as illustrated in FIGS. 1B and 1D.

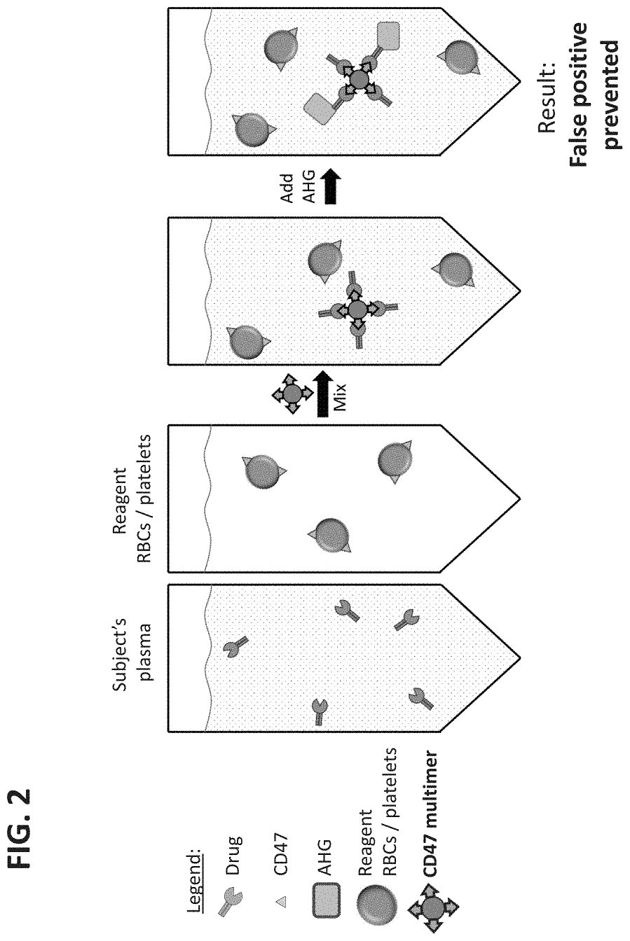

II. Methods of Using a CD47 Multimer that Binds the Drug to Mitigate Interference in a Pre-Transfusion Serological Assay
In some embodiments, the method comprises (a) adding a CD47 multimer that binds a drug (i.e., to the portion of the drug that comprises a moiety that binds to human CD47) to a plasma sample from a subject who has received treatment with the drug, and (b) performing the serological assay of the plasma sample after step (a) using reagent RBCs (i.e., RBCs that are known to express a particular cell surface antigen, or group of cell surface antigens) and/or reagent platelets (i.e., platelets that are known to express a particular cell surface antigen, or group of cell surface antigens), wherein the drug comprises (i) an antibody Fc region and (ii) a moiety that binds to human CD47. Such embodiments are generically depicted in FIG. 2. As shown in FIG. 2, the CD47 multimer binds to the drug (e.g., to the moiety of the drug that binds to human CD47) in the subject's plasma sample and blocks the drug from binding the reagent RBCs and/or reagent platelets. Little or no free drug is available to bind to CD47 on the surface of the reagent RBCs and/or reagent platelets. The interference that would result from the binding of drug to the reagent RBCs and/or reagent platelets (as illustrated in FIG. 1B) is minimized (or, in some embodiments, eliminated), thus preventing a false positive result in the serological assay. In some embodiments, the CD47 multimer is added to the plasma sample to achieve about any one of a 1-fold, 1.5-fold, 2-fold, 2.5-fold, 3-fold, 3.5-fold, 4-fold, 4.5-fold, 5-fold 5.5-fold, 6-fold, 6.5-fold, 7-fold, 7.5-fold, 8-fold, 8.5-fold, 9-fold, 9.5-fold, 10-fold, 10.5-fold, 11-fold, 11.5-fold, 12-fold, 12.5-fold, 13-fold, 13.5-fold, 14-fold, 14.5-fold, or 15-fold molar excess of the anti-SIRP multimer relative to the amount of drug in the plasma. In some embodiments, the CD47 multimer is also added to the reagent RBCs and/or reagent platelets before the serological assay is performed. In some embodiments, the CD47 multimer is added to the reagent RBCs and/or reagent platelets (e.g., only to the reagent RBCs and/or reagent platelets) before the serological assay is performed.

In some embodiments, the method is performed in solution, e.g., wherein the CD47 multimer is soluble. In some embodiments, the CD47 multimer is immobilized to a solid phase before the method is performed via adsorption to a matrix or surface, covalent coupling, or non-covalent coupling. In some embodiments, the CD47 multimer is capable of binding drug following immobilization to the solid phase or solid support. The solid phase or solid support used for immobilization can be any inert support, surface, or carrier that is essentially water insoluble and useful in immunoassays, including supports in the form of, for example, surfaces, particles, porous matrices, cellulose polymer sponge (ImmunoCAP®, Phadia), and the like. Examples of commonly used supports include small sheets, Sephadex, polyvinyl chloride, plastic beads, gold beads, microparticles, assay plates, or test tubes manufactured from polyethylene, polypropylene, polystyrene, and the like. In some embodiments, the CD47 multimer is coated on a microtiter plate, such as a multi-well microtiter plate that can be used to analyze multiple samples simultaneously.

In some embodiments, the moiety of the drug that binds to human CD47 comprises a wild type SIRPα, a SIRPα variant, or a CD47-binding fragment of the wild type SIRPα or the SIRPα variant. In some embodiments, the moiety of the drug that binds to human CD47 comprises the SIRPα variant (or CD47-binding fragment thereof), wherein the SIRPα variant (or CD47-binding fragment thereof) comprises one or more amino acid substitution(s), insertion(s), deletion(s), N-terminal extension(s), and/or C-terminal extension(s) relative to the wild type SIRPα (or CD47-binding fragment thereof). In some embodiments, the moiety of the drug that binds to human CD47 comprises the fragment of the SIRPα variant, and wherein the fragment comprises an extracellular domain of the SIRPα variant. In some embodiments, the CD47 multimer is capable of binding the wild type SIRPα, the SIRPα variant, or the fragment of the wild type SIRPα or the SIRPα variant.

In some embodiments, the moiety of the drug that binds to human CD47 comprises a wild type SIRPγ, a SIRPγ variant, or a CD47-binding fragment of the wild type SIRPγ or the SIRPγ variant. In some embodiments, the moiety of the drug that binds to human CD47 comprises the SIRPγ variant, and wherein the SIRPγ variant comprises one or more amino acid substitution(s), insertion(s), deletion(s), N-terminal extension(s), C-terminal extension(s), or any combination of the preceding, relative to the wild type SIRPγ. In some embodiments, the moiety of the drug that binds to human CD47 comprises a CD47-binding fragment of the SIRPγ variant, and wherein the fragment comprises an extracellular domain of the SIRPγ variant. In some embodiments, the CD47 multimer is capable of binding the wild type SIRPγ, the SIRPγ variant, or the fragment of the wild type SIRPγ or the SIRPγ variant.

In some embodiments, the moiety of the drug that binds to human CD47 comprises a SIRPβ variant that is capable of binding CD47 (e.g., human CD47) or a fragment of the SIRPβ variant that is capable of binding CD47 (e.g., human CD47). In some embodiments, the moiety of the drug that binds to human CD47 comprises the SIRPβ variant, and wherein the SIRPβ variant comprises one or more amino acid substitution(s), insertion(s), deletion(s), N-terminal extension(s), C-terminal extension(s), or any combination of the preceding, relative to the wild type SIRPβ. In some embodiments, the moiety of the drug that binds to human CD47 comprises the fragment of the SIRPβ variant, and wherein the fragment comprises an extracellular domain of the SIRPβ variant and is capable of binding CD47 (e.g., human CD47). In some embodiments, the CD47 multimer is capable of binding the SIRPβ variant or the CD47-binding fragment of the SIRPβ variant.

In some embodiments, the drug comprises an anti-CD47 antibody (or CD47-binding fragment thereof) and the CD47 multimer is capable of binding the anti-CD47 antibody (or CD47-binding fragment thereof).

(a) CD47 Multimers Comprising CD47 Polypeptide Monomers

In some embodiments, the CD47 multimer comprises more than one CD47 polypeptide monomer. In some embodiments, the CD47 multimer comprises at least any one of 2, 3, 4, 5, 6, 7, 8, 9, 10, 15, 20, 25, 30, 35, 40, 45, 50, 55, 60, 65, 70, 75, 80, 85, 90, 95, or up to 100 CD47 polypeptide monomers, including any range in between these values. In some embodiments, a CD47 polypeptide monomer comprises the extracellular domain of a wild type CD47 (“WT CD47-ECD”), or a portion of WT CD47-ECD that is capable of binding the drug and blocking the drug from binding reagent RBCs and/or reagent platelets. In some embodiments, the CD47 polypeptide monomer is a soluble polypeptide that does not comprise the transmembrane domain of CD47 or any portion thereof. In some embodiments, the CD47 polypeptide monomer comprises a fusion polypeptide, e.g., a fusion polypeptide that comprises a CD47 (or a fragment thereof). In some embodiments, the fusion polypeptide comprises a CD47 polypeptide monomer (or a fragment thereof) and, e.g., an antibody Fc domain, such as a murine Fc domain. As discussed in further detail elsewhere herein, in some embodiments, the fusion polypeptide comprises a CD47 polypeptide monomer and a multimerization domain. In some embodiments, the CD47 polypeptide monomer comprises a human CD47, a mouse CD47, a rat CD47, a rhesus CD47, a cynomolgus CD47, or a CD47 of any origin that is capable of binding to the drug and blocking the drug from binding reagent RBCs and/or reagent platelets. In some embodiments, the CD47 polypeptide monomer comprises a fragment of a human CD47, mouse CD47, rat CD47, rhesus CD47, cynomolgus CD47, or CD47 of any origin, provided that the fragment is capable of binding to the drug and blocking the drug from binding reagent RBCs and/or reagent platelets. In some embodiments, the CD47 polypeptide monomer comprises a variant of a wild type CD47 (or a fragment thereof, e.g., a variant of a WT CD47-ECD or a variant of a CD47 that does not comprise the transmembrane domain of CD47, or any portion thereof), provided that the variant is capable of binding to the drug. In some embodiments, the variant (or fragment thereof) comprises one or more amino acid substitution(s), deletion(s), insertion(s), N-terminal addition(s) and/or C-terminal addition(s) relative to a wild type CD47 (e.g., a wild type human, rat, mouse, rhesus, or cynomolgus CD47). In some embodiments, the one or more amino acid substitution(s), deletion(s), insertion(s), N-terminal addition(s) and/or C-terminal addition(s) present in the variant (i.e., the “CD47 variant”) alter the glycosylation pattern of the CD47 variant relative to a wild type CD47 (e.g., a wild type human, rat, mouse, rhesus, or cynomolgus CD47). In some embodiments, the one or more amino acid substitution(s), deletion(s), insertion(s), N-terminal addition(s) and/or C-terminal addition(s) present in the CD47 variant increase the affinity of the CD47 variant for the drug relative to a wild type CD47 (e.g., a wild type human, rat, mouse, rhesus, or cynomolgus CD47). In some embodiments, the affinity of the drug for the CD47 polypeptide monomer is greater than the affinity of the drug for human CD47.

In some embodiments, the CD47 polypeptide monomer comprises a CD47 variant that comprises the amino acid sequence of any one of SEQ ID NOs: 1-6 below:

(SEQ ID NO: 1)
QLLFNKTKSV EFTFSNDTVV IPCFVTNMEA QNTTEVYVKW
KFKGRDIYTF DGALNKSTVP TDFSSAKIEV SQLLKGDASL
KMDKSDAVSH TGNYTCEVTE LTREGETIIE LKYRVVS
(SEQ ID NO: 2)
WQLPLLFNKT KSVEFTFGND TVVIPCFVTN MEAQNTTEVY
VKWKFKGRDI YTFDGDKNKS TVPTDFSSAK IEVSQLLKGD
ASLKMDKSDA VSHTGNYTCE VTELTREGET IIELKYRVVS
(SEQ ID NO: 3)
WQPPLLFNKT KSVEFTFGND TVVIPCFVTN MEAQNTTEVY
VKWKFKGRDI YTFDGQANKS TVPTDFSSAK IEVSQLLKGD
ASLKMDKSDA VSHTGNYTCE VTELTREGET IIELKYRVVS
(SEQ ID NO: 4)
WQPPLLFNKT KSVEFTFCND TVVIPCFVTN MEAQNTTEVY
VKWKFKGRDI YTFDGQANKS TVPTDFSSAK IEVSQLLKGD
ASLKMDKSDA VSHTGNYTCE VTELTREGET IIELKYRVVS
(SEQ ID NO: 5)
WQPPLLFNKT KSVEFTCGND TVVIPCFVTN MEAQNTTEVY
VKWKFKGRDI YTFDGQANKS TVPTDFSSAK IEVSQLLKGD
ASLKMDKSDA VSHTGNYTCE VTELTREGET IIELKYRVVS
SEQ ID NO: 6)
QLLFNKTKSV EFTFSNDTVV IPCFVTNMEA QNTTEVYVKW
KFKGRDIYTF DGALNKSTVP TDFSSAKIEV SQLLKGDASL
KMDKSDAVSH TGNYTCEVTE LTREGETIIE LKYRVVSHHH
HHHGLNDIFE AQKIEWHE

In some embodiments, CD47 multimer comprises SEQ ID NO: 6. In some embodiments, the CD47 multimer is added to the plasma sample (e.g., a plasma sample obtained by a subject who has received treatment with drug) to achieve about any one of a 1-fold, 1.5-fold, 2-fold, 2.5-fold, 3-fold, 3.5-fold, 4-fold, 4.5-fold, 5-fold 5.5-fold, 6-fold, 6.5-fold, 7-fold, 7.5-fold, 8-fold, 8.5-fold, 9-fold, 9.5-fold, or 10-fold molar excess of the CD47 multimer relative to the amount of drug in the plasma.

Additional details regarding exemplary CD47 polypeptide monomers (e.g., including CD47 variants) that can be multimerized and used in the methods described herein are provided in Ho et al. (2015) “′Velcro′ Engineering of High Affinity CD47 Ectodomain as Signal Regulatory Protein a (SIRPα) Antagonists That Enhance Antibody-Dependent Cellular Phagocytosis.” J Biol Chem. 290: 12650-12663 and WO 2016/179399, the contents of which are incorporated herein by reference in their entireties.

In some embodiments, the CD47 polypeptide monomer comprises a fusion polypeptide that comprises a multimerization domain. Exemplary multimerization domains include, but are not limited to, e.g., an Fc monomer, such as a murine Fc monomer, a c-Jun leucine zipper domain, and a c-Fos leucine zipper domain. Each of these multimerization domains is capable of forming a dimer. In some embodiments, the multimerization comprises repeating units of GVGVP (SEQ ID NO: 71), VPGG (SEQ ID NO: 142), APGVGV(SEQ ID NO: 72), GAGAGS (SEQ ID NO: 73), GPGGG (SEQ ID NO: 74), GPGGX wherein X is any amino acid (SEQ ID NO: 123), GPGQQ (SEQ ID NO: 124), GPGGY (SEQ ID NO: 125), GGYGPGS (SEQ ID NO: 75), GAPGAPGSQGAPGLQ (SEQ ID NO: 76), GAPGTPGPQGLPGSP (SEQ ID NO: 77), AKLKLAEAKLELA (SEQ ID NO: 78), PPAKVPEVPEPKKPVPEEKVPVPVPKKPEA (SEQ ID NO: 79), and/or GGFGGMGGGX wherein X is any amino acid (SEQ ID NO: 80). See, e.g., Tatham et al. (2000) Trends in Biochemical Sciences, 25, 567-571; Sanford and Kumar (2005) Current Opinion in Biotechnology, 16, 416-421; and Casal et al. (2014) Future Trends for Recombinant Protein-Based Polymers: The Case Study of Development and Application of Silk-Elastin-Like Polymers. In Kabasci (Ed.) Bio-Based Plastics: Materials and Applications (pp. 311-32) John Wiley & Sons, Ltd.

In some embodiments, the CD47 polypeptide monomer comprises a fusion polypeptide that comprises any one of SEQ ID NOs: 1-6 and any one of SEQ ID NO: 81-83. The amino acid sequences of SEQ ID NOs: 81-83 are set forth below. SEQ ID NO: 81 comprises the CH2 and CH3 domains of a murine IgG1. SEQ ID NO: 82 comprises the CH2 and CH3 domains of a murine IgG1, wherein the CH3 domain comprises an N297A substitution, and wherein the amino acid numbering is according to the EU index of Kabat. SEQ ID NO: 83 comprises the CH2 and CH3 domains of a murine IgG2a.

(SEQ ID NO: 81)
VPRDSGCKPC ICTVPEVSSV FIFPPKPKDV LTITLTPKVT
CVVVDISKDD PEVQFSWFVD DVEVHTAQTQ PREEQFNSTF
RSVSELPIMH QDWLNGKEFK CRVNSAAFPA PIEKTISKTK
GRPKAPQVYT IPPPKEQMAK DKVSLTCMIT DFFPEDITVE
WQWNGQPAEN YKNTQPIMDT DGSYFIYSKL NVQKSNWEAG
NTFTCSVLHE GLHNHHTEKS LSHSPG
(SEQ ID NO: 82)
VPRDSGCKPC ICTVPEVSSV FIFPPKPKDV LTITLTPKVT
CVVVDISKDD PEVQFSWFVD DVEVHTAQTQ PREEQFASTF
RSVSELPIMH QDWLNGKEFK CRVNSAAFPA PIEKTISKTK
GRPKAPQVYT IPPPKEQMAK DKVSLTCMIT DFFPEDITVE
WQWNGQPAEN YKNTQPIMDT DGSYFIYSKL NVQKSNWEAG
NTFTCSVLHE GLHNHHTEKS LSHSPG
(SEQ ID NO: 83)
EPRGPTIKPC PPCKCPAPNL LGGPSVFIFP PKIKDVLMIS
LSPIVTCVVV DVSEDDPDVQ ISWFVNNVEV HTAQTQTHRE
DYNSTLRVVS ALPIQHQDWM SGKEFKCKVN NKDLPAPIER
TISKPKGSVR APQVYVLPPP EEEMTKKQVT LTCMVTDFMP
EDIYVEWTNN GKTELNYKNT EPVLDSDGSY FMYSKLRVEK
KNWVERNSYS CSVVHEGLHN HHTTKSFSRT PG

In some embodiments, the CD47 polypeptide monomer comprises a fusion polypeptide whose amino acid sequence set forth in any one of SEQ ID NOs: 84-86 below.

(SEQ ID NO: 84)
QLLFNKTKSV EFTFSNDTVV IPCFVTNMEA QNTTEVYVKW
KFKGRDIYTF DGALNKSTVP TDFSSAKIEV SQLLKGDASL
KMDKSDAVSH TGNYTCEVTE LTREGETIIE LKYRVVSVPR
DSGCKPCICT VPEVSSVFIF PPKPKDVLTI TLTPKVTCVV
VDISKDDPEV QFSWFVDDVE VHTAQTQPRE EQFNSTFRSV
SELPIMHQDW LNGKEFKCRV NSAAFPAPIE KTISKTKGRP
KAPQVYTIPP PKEQMAKDKV SLTCMITDFF PEDITVEWQW
NGQPAENYKN TQPIMDTDGS YFIYSKLNVQ KSNWEAGNTF
TCSVLHEGLH NHHTEKSLSH SPG
(SEQ ID NO: 85)
QLLFNKTKSV EFTFSNDTVV IPCFVTNMEA QNTTEVYVKW
KFKGRDIYTF DGALNKSTVP TDFSSAKIEV SQLLKGDASL
KMDKSDAVSH TGNYTCEVTE LTREGETIIE LKYRVVSVPR
DSGCKPCICT VPEVSSVFIF PPKPKDVLTI TLTPKVTCVV
VDISKDDPEV QFSWFVDDVE VHTAQTQPRE EQFASTFRSV
SELPIMHQDW LNGKEFKCRV NSAAFPAPIE KTISKTKGRP
KAPQVYTIPP PKEQMAKDKV SLTCMITDFF PEDITVEWQW
NGQPAENYKN TQPIMDTDGS YFIYSKLNVQ KSNWEAGNTF
TCSVLHEGLH NHHTEKSLSH SPG
(SEQ ID NO: 86)
QLLFNKTKSV EFTFSNDTVV IPCFVTNMEA QNTTEVYVKW
KFKGRDIYTF DGALNKSTVP TDFSSAKIEV SQLLKGDASL
KMDKSDAVSH TGNYTCEVTE LTREGETIIE LKYRVVSEPR
GPTIKPSPPC KCPAPNLLGG PSVFIFPPKI KDVLMISLSP
IVTCVVVDVS EDDPDVQISW FVNNVEVHTA QTQTHREDYN
STLRVVSALP IQHQDWMSGK EFKCKVNNKD LPAPIERTIS
KPKGSVRAPQ VYVLPPPEEE MTKKQVTLTC MVTDFMPEDI
YVEWTNNGKT ELNYKNTEPV LDSDGSYFMY SKLRVEKKNW
VERNSYSCSV VHEGLHNHHT TKSFSRTPG

In some embodiments, the CD47 polypeptide monomer (e.g., fusion polypeptide) comprises (e.g., further comprises) an epitope tag. In some embodiments, the epitope tag facilitates multimerization of the CD47 polypeptide monomers. In some embodiments, the tag facilitates the immobilization of the CD47 polypeptide monomers to a solid support (e.g. a bead, a glass slide, etc.). Exemplary epitope tags include, but are not limited to, e.g., HHHHHH (SEQ ID NO: 7), GLNDIFEAQKIEWHE (SEQ ID NO: 8), SRLEEELRRRLTE (SEQ ID NO: 9), KRRWKKNFIAVSAANRFKKISSSGAL (SEQ ID NO: 10), a polyglutamate tag, e.g., EEEEEE (SEQ ID NO: 11), GAPVPYPDPLEPR (SEQ ID NO: 12), DYKDDDDK (SEQ ID NO: 13), YPYDVPDYA (SEQ ID NO: 14), TKENPRSNQEESYDDNES (SEQ ID NO: 15), TETSQVAPA (SEQ ID NO: 16), KETAAAKFERQHMDS (SEQ ID NO: 17), MDEKTTGWRGGHVVEGLAGELEQLRARLEHHPQGQREP (SEQ ID NO: 18), SLAELLNAGLGGS (SEQ ID NO: 19), TQDPSRVG (SEQ ID NO: 20), WSHPQFEK (SEQ ID NO: 21), MASMTGGQQMG (SEQ ID NO: 22), EVHTNQDPLD (SEQ ID NO: 23); GKPIPNPLLGLDST (SEQ ID NO: 24), YTDIEMNRLGK (SEQ ID NO: 25), DLYDDDDK (SEQ ID NO: 26), TDKDMTITFTNKKDAE (SEQ ID NO: 27), AHIVMVDAYKPTK (SEQ ID NO: 28), KLGDIEFIKVNK (SEQ ID NO: 29), KLGSIEFIKVNK (SEQ ID NO: 30), DIPATYEFTDGKHYITNEPIPPK (SEQ ID NO: 31), and DPIVMIDNDKPIT (SEQ ID NO: 32).

In some embodiments, the CD47 polypeptide monomer comprises (e.g., is attached to) a ligand. In some embodiments, the ligand is biotin.

In some embodiments, the CD47 polypeptide monomer comprises the amino acid sequence of SEQ ID NO: 6 (see above). SEQ ID NO: 6 comprises, from N-terminus to C-terminus, the amino acid sequence of WT human CD47, a hexahistidine peptide (i.e., HHHHHH (SEQ ID NO: 7)), and the 15 amino acid tag GLNDIFEAQKIEWHE (SEQ ID NO: 8). GLNDIFEAQKIEWHE (SEQ. ID NO: 8), also known as AVITAG™, is specifically biotinylated by the E. coli biotin ligase BirA. In some embodiment, the CD47 polypeptide monomer comprises the amino acid sequence of SEQ ID NO: G, which comprises, from N-terminus to C-terminus, the amino acid sequence of WT human CD47 and a hexahistidine peptide (i.e., HHHHHH (SEQ ID NO: 7).

In some embodiments, the CD47 multimer is a homomultimer that comprises identical CD47 polypeptide monomers (e.g., between 2 and 100 identical CD47 polypeptide monomers described herein). In some embodiments, the CD47 multimer is a heteromultimer that comprises at least two different CD47 polypeptide monomers (e.g., CD47 polypeptide monomers described herein). CD47 heteromultimers comprising any combination of two or more different CD47 polypeptide monomers are contemplated.

In some embodiments, the CD47 polypeptide monomers in a CD47 multimer are linked via peptide bond to form, e.g., a concatenated chain of CD47 polypeptide monomers. In some embodiments, the CD47 polypeptide monomers in a CD47 multimer are linked via linker peptide. Exemplary linker peptides comprise, but are not limited to, e.g., LSGX₁RX₂X₃SX₄DNH (SEQ ID NO: 127) wherein each of X₁-X₄ is any naturally occurring amino acid; X₁SGSRKX₂RVX₃X₄X₅ (SEQ ID NO: 128) wherein each of X₁-X₅ is any naturally occurring amino acid; SGRXSA (SEQ ID NO: 129) wherein X is any naturally occurring amino acid; LSGX₁RX₂X₃SX₄DNH (SEQ ID NO: 130) wherein each of X₁-X₄ is any naturally occurring amino acid; RX₁X₂X₃RKX₄VX₅X₆GX₇ (SEQ ID NO: 137) wherein each of X₁-X₇ is any naturally occurring amino acid; RQARXVV (SEQ ID NO: 138) wherein X is any naturally occurring amino acid; RX₁X₂RKVX₃G (SEQ ID NO: 87) wherein each of X₁-X₃ is any naturally occurring amino acid; KRRKQGASRKA (SEQ ID NO: 88); LSGX₁RX₂X₃SX₄DNH (SEQ ID NO: 89) wherein each of X₁-X₄ is any naturally occurring amino acid; X₁X₂X₃X₄X₅X₆NX₇X₈X₉ (SEQ ID NO: 90) wherein each of X₁-X₉ is any naturally occurring amino acid; AANXL (SEQ ID NO: 91) wherein X is any naturally occurring amino acid; ATNXL (SEQ ID NO: 139) wherein X is any naturally occurring amino acid; SISQX₁YQRSSX₂X₃ (SEQ ID NO: 92) wherein each of X₁-X₃ is any naturally occurring amino acid; SSKLQ (SEQ ID NO: 93); X₁PX₂X₃LIX₄X₅X₆ (SEQ ID NO: 94) wherein each of X₁-X₆ is any naturally occurring amino acid; GPAX₁GLX₂GX₃ (SEQ ID NO: 95) wherein each of X₁-X₃ is any naturally occurring amino acid; GPLGIAGQ (SEQ ID NO: 96); PVGLIG (SEQ ID NO: 97); HPVGLLAR (SEQ ID NO: 98); X₁X₂X₃VIATX₄X₅X₆X₇ (SEQ ID NO: 99) wherein each of X₁-X₇ is any naturally occurring amino acid; X₁YYVTAX₂X₃X₄X₅ (SEQ ID NO: 100) wherein each of X₁-X₅ is any naturally occurring amino acid; PRFKIIGG (SEQ ID NO: 101); PRFRIIGG (SEQ ID NO: 102); SSRHRRALD (SEQ ID NO: 103); RKSSIIIRMRDVVL (SEQ ID NO: 104); SSSFDKGKYKKGDDA (SEQ ID NO: 105); SSSFDKGKYKRGDDA (SEQ ID NO: 106); IEGR (SEQ ID NO: 141); IDGR (SEQ ID NO: 140); GGSIDGR (SEQ ID NO: 107); PLGLWA (SEQ ID NO: 108); and DVAQFVLT (SEQ ID NO: 109).

In some embodiments, the CD47 polypeptide monomers in a CD47 multimer are linked via linker peptide and at least one spacer. In some embodiments, the spacer comprises 3-200 amino acids. In some embodiments, the spacer comprises one or more glycine and/or serine residues. In certain embodiments, the spacer comprises multiple or repeating motifs comprising GS, GGS, GGGGS (SEQ ID NO: 52), GGSG (SEQ ID NO: 53), or SGGG (SEQ ID NO: 54). In certain embodiments, a spacer comprises multiple or repeating motifs comprising GSGS (SEQ ID NO: 55), GSGSGS (SEQ ID NO: 56), GSGSGSGS (SEQ ID NO: 57), GSGSGSGSGS (SEQ ID NO: 58), or GSGSGSGSGSGS (SEQ ID NO: 59). In some embodiments, the spacer comprises multiple or repeating motifs comprising GGS, e.g., GGSGGS (SEQ ID NO: 60), GGSGGSGGS (SEQ ID NO: 61), and GGSGGSGGSGGS (SEQ ID NO: 136). In some embodiments, the spacer comprises multiple or repeating motifs comprising GGSG (SEQ ID NO:53), GGSGGGSG (SEQ ID NO: 62), or GGSGGGSGGGSG (SEQ ID NO: 63). In some embodiments, the spacer comprises multiple repeats, e.g., between 2 and 10 repeats, of SEQ DI NO: 52. In some embodiments, the spacer comprises GENLYFQSGG (SEQ ID NO: 64), SACYCELS (SEQ ID NO: 65), RSIAT (SEQ ID NO: 66), RPACKIPNDLKQKVMNH (SEQ ID NO: 67), GGSAGGSGSGSSGGSSGASGTGTAGGTGSGSGTGSG (SEQ ID NO: 68), AAANSSIDLISVPVDSR (SEQ ID NO: 69), or GGSGGGSEGGGSEGGGSEGGGSEGGGSEGGGSGGGS (SEQ ID NO: 70).

In some embodiments, the CD47 multimer comprises more than one CD47 polypeptide monomer (e.g., between 2 and 100 CD47 polypeptide monomers) attached to a solid support. In some embodiments, the CD47 polypeptide monomers are attached to the solid support via covalent bond or via non-covalent capture. In some embodiments, the solid support is a gold nanosphere, a gold nanoshell, a magnetic bead, a silica bead, a dextran polymer, a tube, a slide, a gel column, or microtiter wells. In some embodiments, the CD47 multimer comprises more than one CD47 polypeptide monomer (e.g., two or more CD47 polypeptide monomers) and a solid support, wherein each of the CD47 polypeptide monomers comprises an epitope tag or a ligand (e.g., as described above), wherein capture agents are immobilized on the solid support, and wherein the CD47 polypeptide monomers are attached to the solid support via the specific binding of the epitope tags or ligands by the capture agents immobilized on the solid support. In some embodiments, the ligand is biotin and the capture agent is streptavidin. In some embodiments, the CD47 multimer (e.g., homomultimer or heteromultimer) comprises a streptavidin or an avidin bound to 2, 3, or 4 biotinylated CD47 polypeptide monomers. In some embodiments, the biotinylated CD47 polypeptide monomer is generating by biotinylating SEQ ID NO: 6, e.g., as described in the Examples.

(b) Methods of Making CD47 Multimers

(i) Recombinant Production

In some embodiments, a CD47 multimer (e.g., a homomultimer or heteromultimer comprising at least two CD47 polypeptide monomers linked via peptide bond or via linker peptide) is produced by a recombinant host cell. A host cell refers to a vehicle that includes the necessary cellular components, e.g., organelles, needed to express a CD47 multimer described herein from their corresponding nucleic acids. The nucleic acids may be included in nucleic acid vectors that can be introduced into the host cell by conventional techniques known in the art (e.g., transformation, transfection, electroporation, calcium phosphate precipitation, direct microinjection, infection, etc.). The choice of nucleic acid vectors depends in part on the host cells to be used. Generally, host cells are of either prokaryotic (e.g., bacterial) or eukaryotic (e.g., mammalian) origin.

(A) Nucleic Acids, Vectors, and Host Cells

A polynucleotide sequence encoding the amino acid sequence of a CD47 multimer may be prepared by a variety of methods known in the art. These methods include, but are not limited to, oligonucleotide-mediated (or site-directed) mutagenesis and PCR mutagenesis. A polynucleotide molecule encoding a CD47 multimer may be obtained using standard techniques, e.g., gene synthesis. In some embodiments, a polynucleotide molecule encoding a CD47 multimer may be mutated to contain specific substitutions using standard techniques in the art, e.g., QUIKCHANGE™ mutagenesis. Polynucleotides can be synthesized using nucleotide synthesizer or PCR techniques. Polynucleotide sequences encoding a CD47 multimer may be inserted into a vector capable of replicating and expressing the polynucleotides in prokaryotic or eukaryotic host cells. Many vectors are available in the art. Each vector may contain various components that may be adjusted and optimized for compatibility with the particular host cell. For example, the vector components may include, but are not limited to, an origin of replication, a selection marker gene, a promoter, a ribosome binding site, a signal sequence, a polynucleotide sequence encoding a CD47 multimer, and a transcription termination sequence. In some embodiments, a vector can include internal ribosome entry site (IBES) that allows the expression of multiple CD47 multimers. Some examples of bacterial expression vectors include, but are not limited to, pGEX series of vectors (e.g., pGEX-2T, pGEX-3X, pGEX-4T, pGEX-5X, pGEX-6P), pET series of vectors (e.g., pET-21, pET-21a, pET-21b, pET-23, pET-24), pACYC series of vectors (e.g., pACYDuet-1), pDEST series of vectors (e.g., pDEST14, pDEST15, pDEST24, pDEST42), and pBR322 and its derivatives (see, e.g., U.S. Pat. No. 5,648,237). Some examples of mammalian expression vectors include, but are not limited to, pCDNA3, pCDNA4, pNICE, pSELECT, and pFLAG-CMV. Other types of nucleic acid vectors include viral vectors for expressing a protein in a host cell (e.g., baculovirus vectors for expressing proteins in an insect host cell).

In some embodiments, E. coli cells are used as host cells. Examples of E. coli strains include, but are not limited to, e.g., E. coli 294 (ATCC® 31,446), E. coli 2 1776 (ATCC® 31,537), E. coli BL21 (DE3) (ATCC®BAA-1025), and E. coli RV308 (ATCC® 31,608). In some embodiments, mammalian cells are used as host cells. Examples of mammalian cell types include, but are not limited to, e.g., human embryonic kidney (HEK) cells, Chinese hamster ovary (CHO) cells, HeLa cells, PC3 cells, Vero cells, and MC3T3 cells. Different host cells have characteristic and specific mechanisms for the posttranslational processing and modification of protein products. Appropriate cell lines or host systems may be chosen to ensure the correct modification and processing of the protein expressed. The above-described expression vectors may be introduced into appropriate host cells using conventional techniques in the art, e.g., transformation, transfection, electroporation, calcium phosphate precipitation, and direct microinjection. Once the vectors are introduced into host cells for protein production, host cells are cultured in conventional nutrient media modified as appropriate for inducing promoters, selecting transformants, or amplifying the genes encoding the desired sequences.

(B) Protein Production, Recovery, and Purification

Host cells used to produce CD47 multimers may be grown in media known in the art and suitable for culturing of the selected host cells. Examples of suitable media for bacterial host cells include Luria broth (LB) plus necessary supplements, such as a selection agent, e.g., ampicillin. Examples of suitable media for mammalian host cells include Minimal Essential Medium (MEM), Dulbecco's Modified Eagle's Medium (DMEM), DMEM with supplemented fetal bovine serum (FBS), and RPMI-1640. Host cells are cultured at suitable temperatures, such as from about 20° C. to about 39° C., e.g., from 25° C. to about 37° C. The pH of the medium is generally between about 6.8 and about 7.4, e.g., about 7.0, depending mainly on the host organism. If an inducible promoter is used in the expression vector, protein expression is induced under conditions suitable for the activation of the promoter. In some embodiments, recovery of the CD47 multimer produced by the host cell typically involves disrupting the host cell, generally by such means as osmotic shock, sonication, or lysis. Once the cells are disrupted, cell debris may be removed by centrifugation or filtration. In some embodiments, the CD47 multimer is secreted into the culture medium. Supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants. The CD47 multimer may be further purified, for example, by affinity resin chromatography. Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin, SOS-PAGE, and gel filtration.

(ii) Non-Covalent Capture to a Solid Support

In some embodiments, a CD47 multimer (e.g., CD47 homomultimer or CD47 heteromultimer) is produced by attaching more than one CD47 polypeptide monomer (e.g., between 2 and 100 CD47 polypeptide monomers) to a solid support via non-covalent capture. In some embodiments, the solid support is a gold nanosphere, a gold nanoshell, a magnetic bead, a silica bead, a dextran polymer, a tube, a slide, a gel column, microtiter wells. In some embodiments, the solid support comprises or is fabricated from, e.g., glass, cellulose, polyacrylamide, nylon, polystyrene, polyvinyl chloride or polypropylene. In some embodiments, the CD47 polypeptide monomers each comprises a ligand, and capture agents are immobilized on the solid support, and the CD47 multimer is produced by attaching the CD47 polypeptide monomers to the solid support via the specific binding of the ligands by the capture agents immobilized on the solid support. In some embodiments, the ligand is biotin and the capture agent is streptavidin. Also provided herein is a CD47 multimer (e.g., homomultimer or heteromultimer) that comprises at least two CD47 polypeptide monomers immobilized on a solid phase or solid support (e.g., a solid phase or solid support described herein). In some embodiments, the CD47 polypeptide monomers are each biotinylated, streptavidin molecules are immobilized on the solid support, and the CD47 polypeptide monomers are attached to the solid support via the specific binding of the biotin to the avidin.

In some embodiments, a CD47 multimer is prepared by culturing host cell comprising a nucleic acid encoding a CD47 polypeptide monomer that comprises SEQ ID NO: 6 under appropriate conditions to cause expression of the CD47 polypeptide monomer and recovering the CD47 polypeptide monomer. The CD47 polypeptide monomer, which comprises biotin-acceptor peptide amino acid sequence, is biotinylated using E. coli biotin ligase (BirA). Streptavidin and Avidin are tetrameric biotin-binding glycoproteins. Each subunit of streptavidin and avidin is capable of binding biotin with high specificity and high affinity (K_{D}=˜10⁻¹⁵). In some embodiments, the CD47 multimer is produced by attaching biotinylated CD47 polypeptide monomers to a solid support onto which streptavidin or avidin molecules have been immobilized. In some embodiments, provided is a CD47 multimer (e.g., homomultimer or heteromultimer) that comprises more than one CD47 polypeptide monomers (e.g., between 2 and 100 CD47 polypeptide monomers) attached to, e.g., a streptavidin- or avidin-conjugated solid support. Exemplary streptavidin- or avidin-conjugated solid supports include, but are not limited to, e.g., gold beads (available from e.g., Nanocs, Nanocomposix, and Cytodiagnostics), gold nanoshells (also available from e.g., Nanocs, Nanocomposix, and Cytodiagnostics), dextran polymers (available from, e.g., FinaBiosoltions), silica beads (available from, e.g., Bangs Labs, ThermoFisher, Vector Labs), magnetic beads (available from, e.g., CD Bioparticles, ThermoFisher, Sigma Aldrich), and sepharose beads (available from, e.g., BioVision, and CellSignal). In some embodiments, provided is a CD47 multimer (e.g., homomultimer or heteromultimer) comprises a streptavidin or an avidin bound to 2, 3, or 4 biotinylated CD47 polypeptide monomers.

In some embodiments, a CD47 multimer is produced by, e.g. linking two or more CD47 polypeptide monomers to each other via bifunctional protein-coupling agents. Exemplary bifunctional protein coupling agents include, without limitation, e.g., N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bisdiazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene).

In some embodiments, a CD47 multimer is produced by e.g., linking two or more CD47 polypeptide monomers to a solid support via bifunctional agents. Commonly used crosslinking agents include, but are not limited to, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidyl-propionate), bifunctional maleimides such as bis-N-maleimido-1,8-octane and agents such as methyl-3-[(p-azidophenyl)-dithio]propioimidate.

The CD47 multimers produced as discussed above are then used in a method provided herein to prevent interference by a drug that binds CD47 in serological assays.

III. Methods of Using SIRP Multimers that Bind CD47 to Mitigate Interference in a Pre-Transfusion Serological Assay
(a) Methods of Using SIRP Multimers that Bind Human CD47

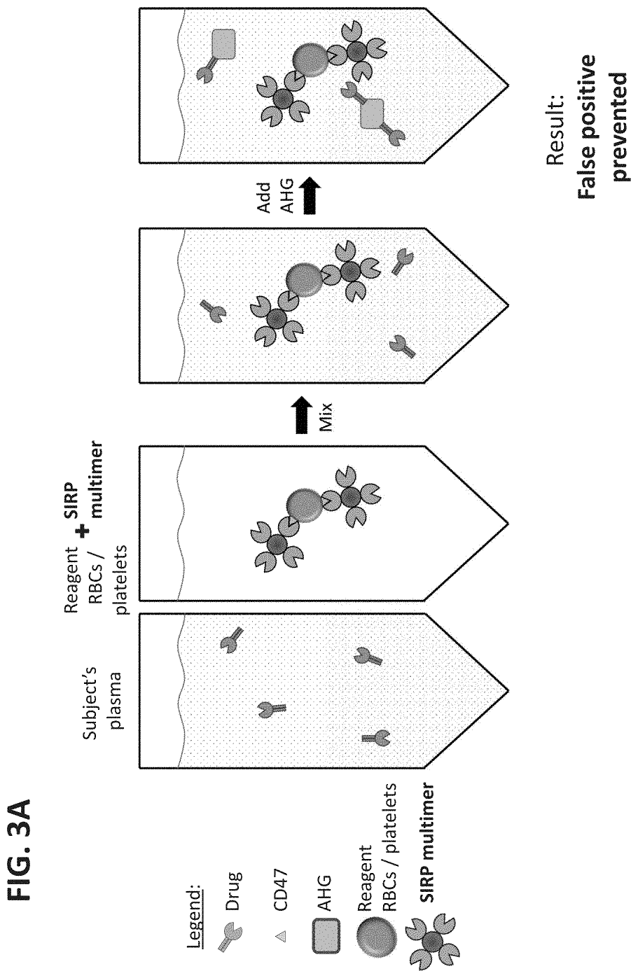

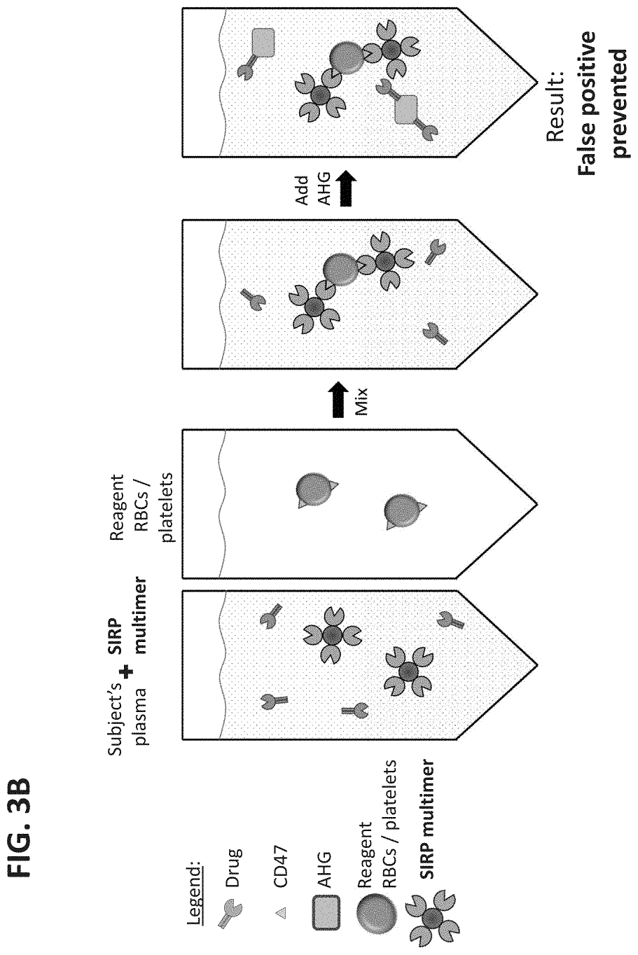

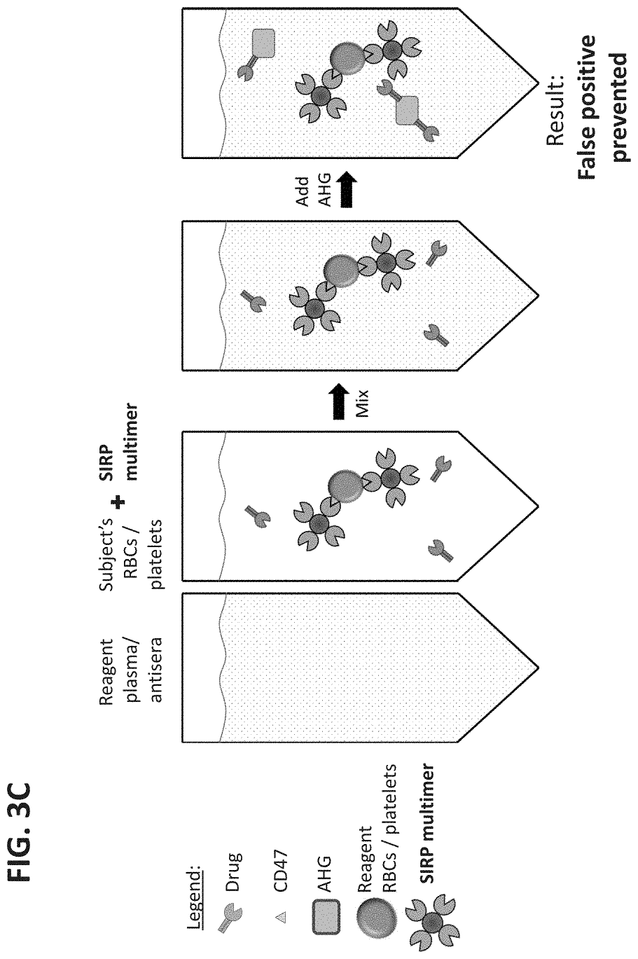

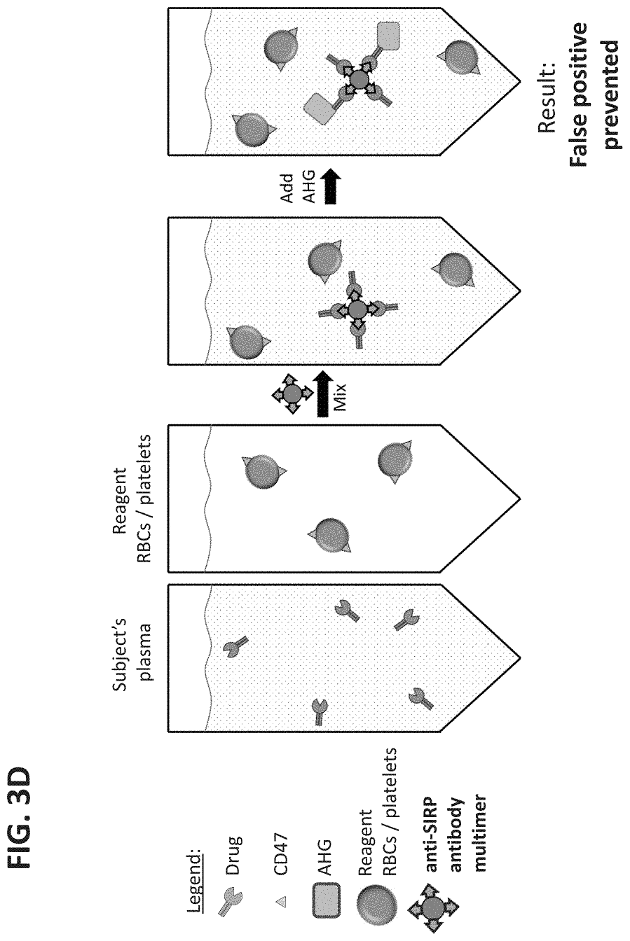

In some embodiments, the method comprises (a) adding a SIRP multimer that binds human CD47 and does not comprise an antibody Fc region that binds to anti-human globulin (AHG) to reagent RBCs (i.e., RBCs that are known to express a particular cell surface antigen, or group of cell surface antigens) and/or reagent platelets (i.e., platelets that are known to express a particular cell surface antigen, or group of cell surface antigens) and (b) performing the serological assay of a plasma sample using the reagent RBCs and/or reagent platelets after step (a), wherein the plasma sample is from a subject who has received treatment with a drug, and wherein the drug comprises (i) an antibody Fc region and (ii) a moiety that binds to human CD47. Such embodiments are generically depicted in FIG. 3A. As shown in FIG. 3A, the SIRP multimer binds the CD47 expressed on the surface of the reagent RBCs (and/or reagent platelets), and blocks the drug from binding the reagent RBCs (and/or reagent platelets), thereby minimizing (or, in some embodiments, eliminating) the interference that would result from the binding of drug to the reagent RBCs and/or reagent platelets (as illustrated in FIG. 1B). In some embodiments, the CD47 multimer is added to the plasma sample to achieve about any one of a 1-fold, 1.5-fold, 2-fold, 2.5-fold, 3-fold, 3.5-fold, 4-fold, 4.5-fold, 5-fold 5.5-fold, 6-fold, 6.5-fold, 7-fold, 7.5-fold, 8-fold, 8.5-fold, 9-fold, 9.5-fold, 10-fold, 10.5-fold, 11-fold, 11.5-fold, 12-fold, 12.5-fold, 13-fold, 13.5-fold, 14-fold, 14.5-fold, or 15-fold molar excess of the SIRP multimer relative to the amount of drug in the plasma sample from the subject. In some embodiments, the SIRP multimer is added to the subject's plasma as well as to the reagent RBCs and/or reagent platelets before the serological assay is performed. In some embodiments, the SIRP multimer is added to the plasma sample to achieve about any one of a 1-fold, 1.5-fold, 2-fold, 2.5-fold, 3-fold, 3.5-fold, 4-fold, 4.5-fold, 5-fold 5.5-fold, 6-fold, 6.5-fold, 7-fold, 7.5-fold, 8-fold, 8.5-fold, 9-fold, 9.5-fold, 10-fold, 10.5-fold, 11-fold, 11.5-fold, 12-fold, 12.5-fold, 13-fold, 13.5-fold, 14-fold, 14.5-fold, or 15-fold molar excess of the anti-SIRP multimer relative to the amount of drug in the plasma.

In some embodiments, the method comprises (a) adding a SIRP multimer that binds to human CD47 and does not comprise an antibody Fc region that binds anti-human globulin (AHG) to a plasma sample from a subject who has received treatment with a drug and (b) performing the serological assay of the plasma sample after step (a) using the reagent RBCs and/or reagent platelets, wherein the drug comprises (i) an antibody Fc region and (ii) a moiety that binds to human CD47. As shown in FIG. 3B, the SIRP multimer competes with the drug for binding to the CD47 expressed on the surface of the reagent RBCs (and/or reagent platelets), thereby minimizing (or, in some embodiments, eliminating) the interference that would result from the binding of drug to the reagent RBCs and/or reagent platelets (as illustrated in FIG. 1B).

In some embodiments, the method comprises (a) adding a SIRP multimer that binds to human CD47 and does not comprise an antibody Fc region that binds to anti-human globulin (AHG) to a blood sample from a subject who has received treatment with a drug and (b) performing the serological assay of the blood sample after step (a) using the reagent plasma/antisera, wherein the drug comprises (i) an antibody Fc region and (ii) a moiety that binds to human CD47. As shown in FIG. 3C, the SIRP multimer competes with the drug for binding to the CD47 expressed on the surface of the subject's RBCs and/or platelets, thereby minimizing (or, in some embodiments, eliminating) the interference that would result from the binding of drug to the subject's RBCs and/or platelets (as illustrated in FIG. 1D). In some embodiments, the SIRP multimer is added to the reagent plasma/antisera as well as to the blood sample from the subject before the serological assay is performed.

In some embodiments, the method is performed in solution, e.g., wherein the SIRP multimer is soluble. In some embodiments, the SIRP multimer is immobilized to a solid phase before the method is performed via adsorption to a matrix or surface, covalent coupling, or non-covalent coupling. In some embodiments, the SIRP multimer is capable of binding CD47 following immobilization to the solid phase or solid support. The solid phase or solid support used for immobilization can be any inert support, surface, or carrier that is essentially water insoluble and useful in immunoassays, including supports in the form of, for example, surfaces, particles, porous matrices, cellulose polymer sponge (ImmunoCAP®, Phadia), and the like. Examples of commonly used supports include small sheets, Sephadex, polyvinyl chloride, plastic beads, gold beads, microparticles, assay plates, or test tubes manufactured from polyethylene, polypropylene, polystyrene, and the like. In some embodiments, the SIRP multimer is coated on a microtiter plate, such as a multi-well microtiter plate that can be used to analyze multiple samples simultaneously.

In some embodiments, the moiety of the drug that binds to human CD47 comprises a wild type SIRPα, a SIRPα variant, or a CD47-binding fragment of the wild type SIRPα or the SIRPα variant. In some embodiments, the moiety of the drug that binds to human CD47 comprises the SIRPα variant (or CD47-binding fragment thereof), wherein the SIRPα variant (or CD47-binding fragment thereof) comprises one or more amino acid substitution(s), insertion(s), deletion(s), N-terminal extension(s), and/or C-terminal extension(s) relative to the wild type SIRPα (or CD47-binding fragment thereof). In some embodiments, the moiety of the drug that binds to human CD47 comprises the fragment of the SIRPα variant, and wherein the fragment comprises an extracellular domain of the SIRPα variant.

In some embodiments, the moiety of the drug that binds to human CD47 comprises a wild type SIRPγ, a SIRPγ variant, or a CD47-binding fragment of the wild type SIRPγ or the SIRPγ variant. In some embodiments, the moiety of the drug that binds to human CD47 comprises the SIRPγ variant, and wherein the SIRPγ variant comprises one or more amino acid substitution(s), insertion(s), deletion(s), N-terminal extension(s), C-terminal extension(s), or any combination of the preceding, relative to the wild type SIRPγ. In some embodiments, the moiety of the drug that binds to human CD47 comprises a CD47-binding fragment of the SIRPγ variant, and wherein the fragment comprises an extracellular domain of the SIRPγ variant.

In some embodiments, the moiety of the drug that binds to human CD47 comprises a SIRPβ variant that is capable of binding CD47 (e.g., human CD47) or a fragment of the SIRPβ variant that is capable of binding CD47 (e.g., human CD47). In some embodiments, the moiety of the drug that binds to human CD47 comprises the SIRPβ variant, and wherein the SIRPβ variant comprises one or more amino acid substitution(s), insertion(s), deletion(s), N-terminal extension(s), C-terminal extension(s), or any combination of the preceding, relative to the wild type SIRPβ. In some embodiments, the moiety of the drug that binds to human CD47 comprises the fragment of the SIRPβ variant, and wherein the fragment comprises an extracellular domain of the SIRPβ variant and is capable of binding CD47 (e.g., human CD47).

In some embodiments, the drug comprises an anti-CD47 antibody.

(b) SIRP Multimers Comprising SIRP Polypeptide Monomers that Bind CD47

In some embodiments, the SIRP multimer that binds human CD47 comprises more than one SIRP polypeptide monomer. In some embodiments, the SIRP multimer comprises at least 2, 3, 4, 5, 6, 7, 8, 9, 10, 15, 20, 25, 30, 35, 40, 45, 50, 55, 60, 65, 70, 75, 80, 85, 90, 95, or up to 100 SIRP polypeptide monomers that are capable of binding CD47 (e.g., human CD47). In some embodiments, “SIRP polypeptide monomer” refers to a SIRPα polypeptide monomer, a SIRPγ polypeptide monomer, or a SIRPβ variant polypeptide monomer that is capable of binding CD47 (e.g., human CD47). In some embodiments, the SIRP polypeptide monomer does not comprise an antibody Fc region. Exemplary SIRPα polypeptide monomers, SIRPγ polypeptide monomers, and SIRPβ variant polypeptide monomers are described in further detail below.

In some embodiments, the SIRP multimer comprises a SIRPα polypeptide monomer that binds human CD47. In some embodiments, the SIRPα polypeptide monomer comprises a fragment of a SIRPα that is capable of binding to CD47 (e.g., the extracellular domain of a wild type SIRPα (“WT SIRPα -ECD”) or the D1 domain thereof). In some embodiments, the fragment of a SIRPα that is capable of binding to CD47 is a soluble fragment (e.g., a fragment of SIRPα that does not include the transmembrane domain or any portion thereof.) In some embodiments, the SIRPα polypeptide monomer comprises a human SIRPα, a mouse SIRPα, a rat SIRPα, a rhesus SIRPα, a cynomolgus SIRPα, or a SIRPα of any origin, provided that the SIRPα is capable of binding to CD47 (e.g., human CD47 expressed on the surface of reagent RBCs and/or reagent platelets). In some embodiments, the SIRPα polypeptide monomer comprises a fragment of a human SIRPα, mouse SIRPα, rat SIRPα, rhesus SIRPα, cynomolgus SIRPα, or a SIRPα of any origin, provided that the fragment is capable of binding to CD47 (e.g., human CD47 expressed on the surface of reagent RBCs and/or reagent platelets). In some embodiments, the SIRPα polypeptide monomer comprises a SIRPα variant (or a fragment thereof, such as a variant of a WT SIRPα-ECD or the D1 domain thereof) that is capable of binding CD47 (e.g., human CD47). In some embodiments, the SIRPα variant (or fragment thereof) that is capable of binding CD47 comprises one or more amino acid substitution(s), deletion(s), insertion(s), N-terminal addition(s) and/or C-terminal addition(s) relative to a wild type SIRPα. In some embodiments, the one or more amino acid substitution(s), deletion(s), insertion(s), N-terminal addition(s) and/or C-terminal addition(s) present in the SIRPα variant (or fragment thereof that is capable of binding CD47) alter the glycosylation pattern of the SIRPα variant relative to a wild type SIRPα. In some embodiments, the one or more amino acid substitution(s), deletion(s), insertion(s), N-terminal addition(s) and/or C-terminal addition(s) present in the SIRPα variant (or fragment of thereof that is capable of binding CD47) increase the affinity of the SIRPα variant (or fragment of thereof that is capable of binding CD47) for human CD47, relative to a wild type SIRPα.

In some embodiments, the SIRP multimer comprises a SIRPγ polypeptide monomer that binds human CD47. In some embodiments, the SIRPγ polypeptide monomer comprises a fragment of a SIRPγ that is capable of binding to CD47 (e.g., the extracellular domain of a wild type SIRPγ (“WT SIRPγ-ECD”) or the D1 domain thereof). In some embodiments, the fragment of a SIRPγ that is capable of binding to CD47 is a soluble fragment (e.g., a fragment of SIRPγ that does not include the transmembrane domain or any portion thereof.) In some embodiments, the SIRPγ polypeptide monomer comprises a human SIRPγ, a mouse SIRPγ, a rat SIRPγ, a rhesus SIRPγ, a cynomolgus SIRPγ, or a SIRPγ of any origin, provided that the SIRPγ is capable of binding to CD47 (e.g., human CD47 expressed on the surface of reagent RBCs and/or reagent platelets). In some embodiments, the SIRPγ polypeptide monomer comprises a fragment of a human SIRPγ, mouse SIRPγ, rat SIRPγ, rhesus SIRPγ, cynomolgus SIRPγ, or SIRPγ of any origin, provided that the fragment is capable of binding to CD47 (e.g., human CD47 expressed on the surface of reagent RBCs and/or reagent platelets). In some embodiments, the SIRPγ polypeptide monomer comprises a SIRPγ variant (or a fragment thereof, such as a variant of a WT SIRPγ-ECD or the D1 domain thereof) that is capable of binding to CD47 (e.g., human CD47 expressed on the surface of reagent RBCs and/or reagent platelets).. In some embodiments, the SIRPγ variant (or fragment thereof that is capable of binding CD47) comprises one or more amino acid substitution(s), deletion(s), insertion(s), N-terminal addition(s) and/or C-terminal addition(s) relative to a wild type SIRPγ. In some embodiments, the one or more amino acid substitution(s), deletion(s), insertion(s), N-terminal addition(s) and/or C-terminal addition(s) present in the SIRPγ variant, (or fragment thereof that is capable of binding CD47) alter the glycosylation pattern of the SIRPγ variant relative to a wild type SIRPγ. In some embodiments, the one or more amino acid substitution(s), deletion(s), insertion(s), N-terminal addition(s) and/or C-terminal addition(s) present in the SIRPγ variant (or fragment of thereof that is capable of binding CD47) increase the affinity of the SIRPγ variant for human CD47, relative to a wild type SIRPγ.

In some embodiments, the SIRP multimer comprises a SIRPβ variant polypeptide monomer that binds human CD47 or a fragment thereof that binds human CD47. In some embodiments, the fragment of a SIRPβ variant polypeptide monomer that is capable of binding to CD47 is a soluble fragment (e.g., a fragment of SIRPβ variant polypeptide that does not include the transmembrane domain or any portion thereof.). In some embodiments, the SIRPβ variant polypeptide monomer comprises a one or more amino acid substitution(s), deletion(s), insertion(s), N-terminal addition(s) and/or C-terminal addition(s) relative to a wild type SIRPβ that increase the affinity of the SIRPβ variant polypeptide monomer for human CD47, relative to a wild type SIRPβ. In some embodiments, the SIRPβ variant polypeptide monomer comprises fragment of a SIRPβ variant that is capable of binding to CD47 (e.g., the extracellular domain of a SIRPβ variant or the D1 domain thereof). In some embodiments, the fragment of a SIRPβ variant that is capable of binding to CD47 is a soluble fragment (e.g., a fragment of a SIRPβ variant that does not include the transmembrane domain or any portion thereof.) In some embodiments, the SIRPβ variant polypeptide monomer comprises a variant of a wild type human SIRPβ, a variant of a wild type mouse SIRPβ, a variant of a wild type rat SIRPβ, a variant of a wild type rhesus SIRPβ, a variant of a wild type cynomolgus SIRPβ, or a SIRPβ variant of any origin, provided that the SIRPβ variant is capable of binding to CD47 (e.g., human CD47 expressed on the surface of reagent RBCs and/or reagent platelets). In some embodiments, the SIRPβ variant polypeptide monomer comprises a fragment of a variant of a wild type human SIRPβ, a variant of a wild type mouse SIRPβ, a variant of a wild type rat SIRPβ a variant of a wild type, a variant of a wild type rhesus SIRPβ, a variant of a wild type cynomolgus SIRPβ, or a SIRPβ variant of any origin, provided that the fragment is capable of binding to CD47 (e.g., human CD47 expressed on the surface of reagent RBCs and/or reagent platelets). In some embodiments, the one or more amino acid substitution(s), deletion(s), insertion(s), N-terminal addition(s) and/or C-terminal addition(s) present in the SIRPβ variant polypeptide monomer (or fragment thereof that is capable of binding CD47) alter the glycosylation pattern of the SIRPβ variant polypeptide monomer relative to a wild type SIRPβ.

In some embodiments, the SIRPα polypeptide monomer, SIRPβ variant polypeptide monomer, or SIRPγ polypeptide monomer comprises the amino acid sequence of any one of SEQ ID NOs: 33-41 below.

(SEQ ID NO: 33)
EEELQIIQPD KSVLVAAGET ATLRCTITSL FPVGPIQWFR
GAGPGRVLIY NQRQGPFPRV TTVSDTTKRN NMDFSIRIGA
ITPADAGTYY CIKFRKGSPD DVEFKSGAGT ELSVRAKPS
(SEQ ID NO: 34)
EEELQIIQPD KSVLVAAGET ATLRCTITSL FPVGPIQWFR
GAGPGRELIY NQREGPFPRV TTVSDTTKRN NMDFSIRIGA
ITPADAGTYY CVKFRKGSPD DVEFKSGAGT ELSVRAKPS
(SEQ ID NO: 35)
EEELQIIQPD KSVLVAAGET ATLRCTITSL FPVGPIQWFR
GAGPGRVLIY NQREGPFPRV TTVSDTTKRN NMDFSIRIGA
ITPADAGTYY CIKFRKGSPD DVEFKSGAGT ELSVRAKPS
(SEQ ID NO: 36)
EDELQIIQPE KSVSVAAGES ATLRCAITSL FPVGPIQWFR
GAGAGRVLIY NQRQGPFPRV TTVSETTKRN NLDFSISISN
ITPADAGTYY CIKFRKGSPD DVEFKSGAGT ELSVRAKPS
(SEQ ID NO: 37)
EEELQIIQPD KSISVAAGES ATLHCTITSL FPVGPIQWFR
GAGPGRVLIY NQRQGPFPRV TTVSDTTKRN NMDFSIRISN
ITPADAGTYY CIKFRKGSPD DVEFKSGAGT ELSVRAKPS
(SEQ ID NO: 38)
EEELQIIQPE KLLLVTVGKT ATLHCTITSL FPVGPIQWFR
GVGPGRVLIY NQRDGPFPRV TTVSDGTKRN NMDFSIRISS
ITPADVGTYY CVKFRKGTPE DVEFKSGPGT EMALGAKPS
(SEQ ID NO: 39)
EEELQIIQPE KLLLVTVGKT ATLHCTITSL FPVGPIQWFR
GVGPGRVLIY NQKDGPFPRV TTVSDGTKRN NMDFSIRISS
ITPADVGTYY CVKFRKGSPE DVEFKSGPGT EMALGAKPS
(SEQ ID NO: 40)
EEELQIIQPE KLLLVTVGKT ATLHCTITSL FPVGPIQWFR
GVGPGRVLIY NQKDGHFPRV TTVSDGTKRN NMDFSIRISS
ITPADVGTYY CVKFRKGSPE DVEFKSGPGT EMALGAKPS
(SEQ ID NO: 41)
EEELQIIQPE KLLLVTVGKT ATLHCTITSH FPVGPIQWFR
GVGPGRVLIY NQKDGHFPRV TTVSDGTKRN NMDFSIRISS
ITPADVGTYY CVKFRKGSPE DVEFKSGPGT EMALGAKPS
(SEQ ID NO: 42)
EEELQIIQPE KLLLVTVGKT ATLHCTITSL FPVGPVLWFR
GVGPGRVLIY NQRQGPFPRV TTVSDTTKRN NMDFSIRISS
ITPADVGTYY CVKFRKGTPE DVEFKSGPGT EMALGAKPS
(SEQ ID NO: 43)
EEELQIIQPE KLLLVTVGKT ATLHCTITSL FPVGPIQWFR
GVGPGRELIY NAREGRFPRV TTVSDLTKRN NMDFSIRISS
ITPADVGTYY CVKFRKGSPE DVEFKSGPGT EMALGAKPS
(SEQ ID NO: 44)
EEELQIIQPE KLLLVTVGKT ATLHCTITSL LPVGPIQWFR
GVGPGRELIY NQRDGPFPRV TTVSDGTKRN NMDFSIRISS
ITPADVGTYY CVKFRKGTPE DVEFKSGPGT EMALGAKPS
(SEQ ID NO: 45)
EEELQIIQPD KSVLVAAGET ATLRCTITSL FPVGPIQWFR
GAGPGRVLIY NQRQGPFPRV TTVSDTTKRN NMDFSIRIGN
ITPADAGTYY CIKFRKGSPD DVEFKSGAGT ELSVRAKPS

Additional details regarding exemplary SIRPα polypeptide monomers (e.g., SIRPα variants), SIRPβ variant polypeptide monomers, and SIRPγ polypeptide monomers (e.g., SIRPγ variants) that can be multimerized and used in the methods described herein are provided in WO 2013/109752; US 2015/0071905; U.S. Pat. No. 9,944,911; WO 2016/023040; WO 2017/027422; US 2017/0107270; U.S. Pat. Nos. 10,259,859; 9,845,345; WO2016187226; US20180155405; WO2017177333; WO2014094122; US2015329616; US20180312563; WO2018176132; WO2018081898; WO2018081897; US20180141986A1; and EP3287470A1, the contents of which are incorporated herein by reference in their entireties.

In some embodiments, the SIRP polypeptide monomer comprises a fusion polypeptide that comprises a multimerization domain, e.g., including, but not limited to the exemplary multimerization domains discussed above for CD47 polypeptide monomers.

In some embodiments, the SIRP polypeptide monomer comprises a fusion polypeptide that comprises any one of SEQ ID NOs: 33-45 and a multimerization domain set forth in any one of SEQ ID NO: 81-83. In some embodiments, the SIRP polypeptide monomer comprises a fusion polypeptide that comprises a SIRP polypeptide monomer disclosed in any one of WO 2013/109752; US 2015/0071905; U.S. Pat. No. 9,944,911; WO 2016/023040; WO 2017/027422; US 2017/0107270; U.S. Pat. Nos. 10,259,859; 9,845,345; WO2016187226; US20180155405; WO2017177333; WO2014094122; US2015329616; US20180312563; WO2018176132; WO2018081898; WO2018081897; US20180141986A1; and EP3287470A1 and a multimerization domain set forth in any one of SEQ ID NOs: 81-83. In some embodiments, the SIRP polypeptide monomer comprises a fusion polypeptide that comprises the amino acid sequence set forth in SEQ ID NO: 110.

(SEQ ID NO: 110)
EEELQIIQPD KSVLVAAGET ATLRCTITSL FPVGPIQWFR
GAGPGRVLIY NQRQGPFPRV TTVSDTTKRN NMDFSIRIGN
ITPADAGTYY CIKFRKGSPD DVEFKSGAGT ELSVRAKPSA
KTTAPSVYPL APVCGDTTGS SVTLGCLVKG YFPEPVTLTW
NSGSLSSGVH TFPAVLQSDL YTLSSSVTVT SSTWPSQSIT
CNVAHPASST KVDKKIEPRG PTIKPCPPCK CPAPNLLGGP
SVFIFPPKIK DVLMISLSPI VTCVVVDVSE DDPDVQISWF
VNNVEVHTA QTQTHREDYN STLRVVSALP IQHQDWMSGK
EFKCKVNNKD LPAPIERTIS KPKGSVRAPQ VYVLPPPEEE
MTKKQVTLTC MVTDFMPEDI YVEWTNNGKT ELNYKNTEPV
LDSDGSYFMY SKLRVEKKNW VERNSYSCSV VHEGLHNHHT
TKSFSRTPG

In some embodiments, the SIRP polypeptide monomer (e.g., fusion polypeptide) comprises (e.g., further comprises) an epitope tag. In some embodiments, the epitope tag facilitates multimerization of the SIRP polypeptide monomers. In some embodiments, the tag facilitates the immobilization of the SIRP polypeptide monomers to a solid support (e.g. beads, glass sides, etc.). Exemplary epitope tags include, but are not limited to SEQ ID NOs: 7-32 described above for CD47 polypeptide monomers.

In some embodiments, the SIRP polypeptide monomer comprises (e.g., is attached to) a ligand. In some embodiments, the ligand is biotin.

In some embodiments, the SIRP polypeptide monomer comprises the amino acid sequence of SEQ ID NO: 111 (see below). SEQ ID NO: 111 comprises, from N-terminus to C-terminus, the amino acid sequence of SEQ ID NO: 45, a hexahistidine peptide (i.e., HHHHHH (SEQ ID NO: 7)), and the 15 amino acid tag GLNDIFEAQKIEWHE (SEQ ID NO: 8). GLNDIFEAQKIEWHE (SEQ ID NO: 8), also known as AVITAG™, is specifically biotinylated by the E. coli biotin ligase BirA. In some embodiment, the CD47 polypeptide monomer comprises the amino acid sequence of SEQ ID NO: 112 (see below), which comprises, from N-terminus to C-terminus, the amino acid sequence of SEQ ID NO: 45 and a hexahistidine peptide (i.e., HHHHHH (SEQ ID NO: 7).

(SEQ ID NO: 111)
EEELQIIQPD KSVLVAAGET ATLRCTITSL FPVGPIQWFR
GAGPGRVLIY NQRQGPFPRV TTVSDTTKRN NMDFSIRIGN
ITPADAGTYY CIKFRKGSPD DVEFKSGAGT ELSVRAKPSH
HHHHHGLNDI FEAQKIEWHE
(SEQ ID NO: 112)
EEELQIIQPD KSVLVAAGET ATLRCTITSL FPVGPIQWFR
GAGPGRVLIY NQRQGPFPRV TTVSDTTKRN NMDFSIRIGN
ITPADAGTYY CIKFRKGSPD DVEFKSGAGT ELSVRAKPSH
HHHHH

In some embodiments, the SIRP multimer that binds human CD47 is a homomultimer that comprises identical SIRP polypeptide monomers (e.g., identical SIRPα polypeptide monomers, identical SIRPβ variant polypeptide monomers, or identical SIRPγ polypeptide monomers, such as SIRP polypeptide monomers described herein). In some embodiments, the SIRP multimer that binds human CD47 is a heteromultimer that comprises at least two different SIRPα polypeptide monomers, two different SIRPβ variant polypeptide monomers, two different SIRPγ polypeptide monomers, or combinations of any of the foregoing. SIRP heteromultimers comprising any combination of two or more different SIRPα polypeptide monomers, SIRPβ variant polypeptide monomers, and/or SIRPγ polypeptide monomers (e.g., SIRP polypeptide monomers described herein) are contemplated.

In some embodiments, the SIRP polypeptide monomers in a SIRP multimer are linked via peptide bond or linker peptide to form, e.g., a concatenated chain of SIRP polypeptide monomers. In some embodiments, the SIRP polypeptide monomer in a SIRP multimer are linked via linker peptide. Exemplary linker peptides comprise, but are not limited to, those set forth in SEQ ID NO: 85-109, 127-130, 140, and 141, and other linkers that find use with CD47 multimers (see above). In some embodiments, the SIRP polypeptide monomers in a SIRP multimer are linked via peptide linker and at least one spacer. Exemplary peptide spacers include, but are not limited to, SEQ ID NOs: 52-70 and other spacers that find use with CD47 multimers (see above).

In some embodiments, the SIRP multimer that binds human CD47 comprises more than one SIRP polypeptide monomer (e.g., between 2 and 100 SIRP polypeptide monomers) attached to a solid support. In some embodiments, the SIRP polypeptide monomers are attached to the solid support via covalent bond or via non-covalent capture. In some embodiments, the solid support is a gold nanosphere, a gold nanoshell, a magnetic bead, a silica bead, a dextran polymer, a tube, a slide, a gel column, or microtiter wells. In some embodiments, the solid support comprises or is fabricated from, e.g., glass, cellulose, polyacrylamide, nylon, polystyrene, polyvinyl chloride or polypropylene. In some embodiments, SIRP multimer that binds human CD47 comprises more than one SIRP polypeptide monomer (e.g., two or more SIRP polypeptide monomers) and a solid support, wherein each of the SIRP polypeptide monomers comprises an epitope tag or ligand (e.g., as described above), wherein capture agents are immobilized on the solid support, and wherein the SIRP polypeptide monomers are attached to the solid support via the specific binding of the epitope tags or ligands by the capture agents immobilized on the solid support. In some embodiments, the ligand is biotin and the capture agent is streptavidin. In some embodiments, the SIRP multimer (e.g., homomultimer or heteromultimer) comprises a streptavidin or an avidin bound to 2, 3, or 4 biotinylated SIRP polypeptide monomers.

(c) Methods of Making SIRP Multimers.

In some embodiments, a SIRP multimer (e.g., a homomultimer or heteromultimer comprising at least two SIRP polypeptide monomers linked via peptide bond or via linker peptide) is produced by a recombinant host cell. Any of the methods described herein for producing a CD47 multimer using recombinant techniques are applicable for the production of a SIRP multimer comprising SIRP polypeptide monomers, as well.

In some embodiments, a SIRP multimer (e.g., homomultimer or heteromultimer) is produced by attaching more than one SIRP polypeptide monomers (e.g., between 2 and 100 SIRP polypeptide monomers) to a solid support via non-covalent capture. In some embodiments, the solid support is a gold nanosphere, a gold nanoshell, a magnetic bead, a silica bead, a dextran polymer, a tube, a slide, a gel column, or microtiter wells. In some embodiments, the SIRP polypeptide monomers each comprise a ligand, and capture agents are immobilized on the solid support, and the SIRP multimer is produced by attaching the SIRP polypeptide monomers to the solid support via the specific binding of the ligands by the capture agents immobilized on the solid support. In some embodiments, the ligand is biotin and the capture agent is streptavidin.

Any of the methods described elsewhere herein for attaching CD47 polypeptide monomers to a solid support to produce a CD47 multimer are applicable for attaching SIRP polypeptide monomers to a solid support to produce a SIRP multimer, as well. For example, in some embodiments, a SIRP multimer is prepared by culturing host cell comprising a nucleic acid encoding a SIRP polypeptide monomer that comprises SEQ ID NO: 111 under appropriate conditions to cause expression of the SIRP polypeptide monomer and recovering the SIRP polypeptide monomer. The SIRP polypeptide monomer, which comprises biotin-acceptor peptide amino acid sequence, is biotinylated using E. coli biotin ligase (BirA). In some embodiments, the SIRP multimer is produced by attaching biotinylated SIRP polypeptide monomers to a solid support onto which streptavidin or avidin molecules have been immobilized. In some embodiments, provided is a SIRP multimer (e.g., homomultimer or heteromultimer) that comprises more than one SIRP polypeptide monomer (e.g., between 2 and 100 SIRP polypeptide monomers) attached to, e.g., a streptavidin- or avidin-conjugated solid support. Exemplary streptavidin- or avidin-conjugated solid supports are described in further detail elsewhere herein. In some embodiments, provided is a SIRP multimer (e.g., homomultimer or heteromultimer) comprises a streptavidin or an avidin bound to 2, 3, or 4 biotinylated SIRP polypeptide monomers.

In some embodiments, the SIRP multimer is produced by linking one or more SIRP monomer polypeptides to, e.g., a solid support, or, e.g., to each other, using bifunctional crosslinkers (e.g., such as those described elsewhere herein).

The SIRP multimers thus produced are then used in a method provided herein to prevent interference by a drug that binds CD47 in serological assays.

### IV. Methods of Using Anti-SIRP Multimers to Mitigate Interference in a Pre-Transfusion

Serological Assay

(a) Methods of Using Anti-SIRP Multimers that Bind the Drug

In some embodiments, the method comprises (a) adding an anti-SIRP multimer that binds a drug (i.e., to the portion of the drug that comprises a moiety that binds to human CD47) to a plasma sample from a subject who has received treatment with the drug, and (b) performing the serological assay of the plasma sample after step (a) using reagent RBCs (i.e., RBCs that are known to express a particular cell surface antigen, or group of cell surface antigens) and/or reagent platelets (i.e., platelets that are known to express a particular cell surface antigen, or group of cell surface antigens), wherein the drug comprises (i) an antibody Fc region and (ii) a moiety that binds to human CD47. Such embodiments are generically depicted in FIG. 3D. As shown in FIG. 3D, the anti-SIRP multimer binds to the drug (e.g., to the moiety of the drug that binds to human CD47) in the subject's plasma sample and blocks the drug from binding the reagent RBCs and/or reagent platelets. Little or no free drug available to bind to CD47 on the surface of the reagent RBCs and/or reagent platelets. The interference that would result from the binding of drug to the reagent RBCs and/or reagent platelets (as illustrated in FIG. 1B) is minimized (or, in some embodiments, eliminated), thus preventing a false positive result in the serological assay. In some embodiments, the anti-SIRP multimer is added to the plasma sample to achieve about any one of 1-fold, 1.5-fold, 2-fold, 2.5-fold, 3-fold, 3.5-fold, 4-fold, 4.5-fold, 5-fold 5.5-fold, 6-fold, 6.5-fold, 7-fold, 7.5-fold, 8-fold, 8.5-fold, 9-fold, 9.5-fold, 10-fold, 10.5-fold, 11-fold, 11.5-fold, 12-fold, 12.5-fold, 13-fold, 13.5-fold, 14-fold, 14.5-fold, or 15-fold molar excess of the anti-SIRP multimer relative to the amount of drug in the plasma. In some embodiments, the anti-SIRP multimer that binds to the drug is also added to the reagent RBCs and/or reagent platelets before the serological assay is performed. In some embodiments, the anti-SIRP multimer is added to the reagent RBCs and/or reagent platelets (e.g., only to the reagent RBCs and/or reagent platelets) before the serological assay is performed.

In some embodiments, the method is performed in solution, e.g., wherein the anti-SIRP multimer that binds to the drug is soluble. In some embodiments, the anti-SIRP multimer that binds to the drug is immobilized to a solid phase before the method is performed via adsorption to a matrix or surface, covalent coupling, or non-covalent coupling. In some embodiments, the anti-SIRP multimer that binds to the drug is capable of binding drug following immobilization to the solid phase or solid support. The solid phase or solid support used for immobilization can be any inert support, surface, or carrier that is essentially water insoluble and useful in immunoassays, including supports in the form of, for example, surfaces, particles, porous matrices, cellulose polymer sponge (ImmunoCAP®, Phadia), and the like. Examples of commonly used supports include small sheets, Sephadex, polyvinyl chloride, plastic beads, gold beads, microparticles, assay plates, or test tubes manufactured from polyethylene, polypropylene, polystyrene, and the like. In some embodiments, the anti-SIRP multimer that binds to the drug is coated on a microtiter plate, such as a multi-well microtiter plate that can be used to analyze multiple samples simultaneously.

In some embodiments, the moiety of the drug that binds to human CD47 comprises a wild type SIRPα, a SIRPα variant, or a CD47-binding fragment of the wild type SIRPα or the SIRPα variant. In some embodiments, the moiety of the drug that binds to human CD47 comprises the SIRPα variant (or CD47-binding fragment thereof), wherein the SIRPα variant (or CD47-binding fragment thereof) comprises one or more amino acid substitution(s), insertion(s), deletion(s), N-terminal extension(s), and/or C-terminal extension(s) relative to the wild type SIRPα (or CD47-binding fragment thereof). In some embodiments, the moiety of the drug that binds to human CD47 comprises the fragment of the SIRPα variant, and wherein the fragment comprises an extracellular domain of the SIRPα variant.

In some embodiments, the moiety of the drug that binds to human CD47 comprises a wild type SIRPγ, a SIRPγ variant, or a CD47-binding fragment of the wild type SIRPγ or the SIRPγ variant. In some embodiments, the moiety of the drug that binds to human CD47 comprises the SIRPγ variant, and wherein the SIRPγ variant comprises one or more amino acid substitution(s), insertion(s), deletion(s), N-terminal extension(s), C-terminal extension(s), or any combination of the preceding, relative to the wild type SIRPγ. In some embodiments, the moiety of the drug that binds to human CD47 comprises a CD47-binding fragment of the SIRPγ variant, and wherein the fragment comprises an extracellular domain of the SIRPγ variant.

In some embodiments, the moiety of the drug that binds to human CD47 comprises a SIRPβ variant that is capable of binding CD47 (e.g., human CD47) or a fragment of the SIRPβ variant that is capable of binding CD47 (e.g., human CD47). In some embodiments, the moiety of the drug that binds to human CD47 comprises the SIRPβ variant, and wherein the SIRPβ variant comprises one or more amino acid substitution(s), insertion(s), deletion(s), N-terminal extension(s), C-terminal extension(s), or any combination of the preceding, relative to the wild type SIRPβ. In some embodiments, the moiety of the drug that binds to human CD47 comprises the fragment of the SIRPβ variant, and wherein the fragment comprises an extracellular domain of the SIRPβ variant and is capable of binding CD47 (e.g., human CD47).

(b) Anti-SIRP Multimers

In some embodiments, the anti-SIRP multimer that binds to the drug comprises an anti-SIRP antibody (or drug binding fragment thereof) capable of binding the SIRPα, the SIRPα variant, the SIRPβ variant, the SIRPγ, or the SIRPγ variant portion of the drug. In some embodiments, the anti-SIRP multimer that binds to the drug comprises one or more anti-SIRP antibodies (or drug binding fragments thereof) capable of binding the SIRPα, the SIRPα variant, the SIRPβ variant, the SIRPγ, or the SIRPγ variant portion of the drug. In some embodiments, the anti-SIRP multimer comprises at least 1, 2, 3, 4, 5, 6, 7, 8, 9, 10, 15, 20, 25, 30, 35, 40, 45, 50, 55, 60, 65, 70, 75, 80, 85, 90, 95, or up to 100 anti-SIRP antibodies (or drug binding fragments thereof). In some embodiments, the anti-SIRP multimer comprises (e.g., is) a bivalent anti-SIRP antibody. In some embodiments, “anti-SIRP antibody” refers to an antibody or drug binding fragment thereof (e.g., antigen binding fragment thereof) that specifically binds a SIRPα, a SIRPα variant, a SIRPγ, a SIRPγ variant, or a SIRPβ variant. The extracellular domains of SIRPα, SIRPβ, and SIRPγ are highly homologous. Thus, in some embodiments, “anti-SIRP antibody” refers to an antibody or drug binding fragment thereof that is capable of cross reacting with one or more of a SIRPα, a SIRPα variant, a SIRPγ, a SIRPγ variant, and/or a SIRPβ variant.

In some embodiments, the anti-SIRP multimer comprises a full length anti-SIRP antibody. In some embodiments, the anti-SIRP antibody comprises an Fc region (or a portion thereof) that does not bind to anti-human globulin reagent (AHG). (Further details regarding serological assays, and reagents used in such assays, are provided elsewhere herein.) In some embodiments, the anti-SIRP antibody comprises a murine Fc region (or portion thereof). In some embodiments, the murine Fc region comprises an amino acid sequence set forth in any one of SEQ ID NOs: 81-83. In some embodiments, the drug binding fragment (e.g., antigen binding fragment) of the anti-SIRP antibody is, e.g., without limitation, a Fab, a Fab′, an F(ab′)₂, a Fab′-SH, an Fv, a diabody, a one-armed antibody, an scFv, an scFv-Fc, a single domain antibody, a single heavy chain antibody, etc. In some embodiments, the anti-SIRP antibody (or drug binding fragment thereof) is an ADA (anti-drug antibody) or a NAb (neutralizing antibody) that binds to the drug (i.e., the portion of the drug that comprises the SIRPα, the SIRPα variant, the SIRPβ variant, the SIRPγ, or the SIRPγ variant). In some embodiments, the affinity of the drug for the anti-SIRP antibody is greater than the affinity of the drug for human CD47. In some embodiments, the anti-SIRP antibody (or drug binding fragment thereof) comprises a heavy chain variable domain (VH) that comprises the amino acid sequence of SEQ ID NO: 46 and a light chain variable domain (VL) that comprises the amino acid sequence of SEQ ID NO: 47. In some embodiments, the anti-SIRP antibody (or drug binding fragment thereof) comprises a heavy chain variable domain (VH) that comprises the amino acid sequence of SEQ ID NO: 48 and a light chain variable domain (VL) that comprises the amino acid sequence of SEQ ID NO: 49. In some embodiments, the anti-SIRP antibody (or drug binding fragment thereof) comprises a heavy chain variable domain (VH) that comprises the amino acid sequence of SEQ ID NO: 50 and a light chain variable domain (VL) that comprises the amino acid sequence of SEQ ID NO: 51. In some embodiments, the anti-SIRP antibody (or drug binding fragment thereof) comprises a heavy chain variable domain (VH) that comprises the amino acid sequence of SEQ ID NO: 113 and a light chain variable domain (VL) that comprises the amino acid sequence of SEQ ID NO: 114. In some embodiments, the anti-SIRP antibody (or drug binding fragment thereof) comprises a heavy chain variable domain (VH) that comprises the amino acid sequence of SEQ ID NO: 115 and a light chain variable domain (VL) that comprises the amino acid sequence of SEQ ID NO: 116. In some embodiments, the anti-SIRP antibody (or drug binding fragment thereof) comprises a heavy chain variable domain (VH) that comprises the amino acid sequence of SEQ ID NO: 133 and a light chain variable domain (VL) that comprises the amino acid sequence of SEQ ID NO: 134. In some embodiments, the anti-SIRP antibody comprises a murine Fc domain that comprises an amino acid sequence set forth in any one of SEQ ID NOs: 81-83. In some embodiments, the drug binding fragment of the anti-SIRP antibody comprises, e.g., a Fab, a Fab′, an F(ab′)2, a Fab′-SH, an Fv, a diabody, a one-armed antibody, an scFv, an scFv-Fc, a single domain antibody, a single heavy chain antibody, etc. In some embodiments, the drug binding fragment of the anti-SIRP antibody comprises a Fab or F(ab′)2 that comprises SEQ ID NO: 131 and SEQ ID NO: 132.

(SEQ ID NO: 46)
DVQLVESGGG VVRPGESLRL SCAASGFSFS SYAMNWVRQA
PGEGLEWVSR INSGGGGTDY AESVKGRFTI SRDNSENTLY
LQMNSLRAED TAVYYCAKQY DWNSFFDYWG LGALVTVSS
(SEQ ID NO: 47)
ETVLTQSPAT LSVSPGERAT LSCRASQTVG SKLAWHQQKP
GQAPRLLIYD ATNRATGISD RFSGSGSGTD FTLTISSLQT
EDSAVYYCQQ YYYWPPYRFG GGTKVEIK
(SEQ ID NO: 48)
DVQLVESGGG VVRPGESLRL SCEASGFTFS SNAMSWVRQA
PGKGLEWVAG ISSGSDTYYG DSVKGRLTIS RDNSKNILYL
QMNSLTAEDT AVYYCARETW NHLFDYWGQG TLVTVSS
(SEQ ID NO: 49)
SYELTQPPSV SVSPGQTARI TCSGGSYSSY YYAWYQQKPG
QAPVTLIYSD DKRPSNIPER FSGSSSGTTV TLTISGVQAE
DEADYYCGGY DQSSYTNPFG GGTKLTVL
(SEQ ID NO: 50)
DVQLVESGGG VVRPGESLRL SCAASGFTFS SYDMNWVRQA
PGEGLEWVSL ISGSGEIIYY ADSVKGRFTI SRDNSKNTLY
LQMNSLRAED TAVYYCAKEN NRYRFFDDWG QGTLVTVSS
(SEQ ID NO: 51)
ETVLTQSPGT LTLSPGERAT LTCRASQSVY TYLAWYQEKP
GQAPRLLIYG ASSRATGIPD RFSGSGSGTE FTLTISSLQS
EDFAVYYCQQ YYDRPPLTFG GGTKVEIK
(SEQ ID NO: 113)
DVQLVESGGG VVRPGESLRL SCAASGFTFS SYAMSWVRQA
PGKGLEWLAG ISAGGSDTYY IDSVKGRFTI SRDNPKNSLY
LQMSSLTAED TAVYYCARET WNHLFDYWGL GTLVTVSS
(SEQ ID NO: 114)
ALTQPASVSA NPGETVKITC SGGDYYSTYY AWYQQKSPGS
APVTVIHSDD KRPSDIPSRF SGSASGSAAT LIITGVRVED
EAVYYCGGYD GRTYINTFGA GTTLTVL
(SEQ ID NO: 115)
DVQLVESGGG VVRPGESLRL SCAASGFTFS SNAMSWVRQA
PGKGLEWLAG ISAGGSDTYY PASVKGRFTI SRDNSKNTLY
LQMNTLTAED TAVYYCARET WNHLFDYWGL GTLVTVSS
(SEQ ID NO: 116)
ALTQPASVSA NPGETVKIAC SGGDYYSYYY GWYQQKAPGS
ALVTVIYSDD KRPSDIPSRF SGSASGSTAT LTITGVRAED
EAVYYCGGYD YSTYANAFGA GTTLTVL
(SEQ ID NO: 133)
DVQLVESGGG VVRPGESLRL SCEASGFTFS SNAMSWVRQA
PGKGLEWVAG ISSGSDTYYG DSVKGRLTIS RDNSKNILYL
QMNSLTAEDT AVYYCARETW NHLFDYWGLG TLVTVS
(SEQ ID NO: 134)
ALTQPASVSA SPGETVEITC SGGSDSSYYY GWYQQKSPGS
APVTVIYSDN KRPSNIPSRF SGSASGSTAT LTITGVRVED
EAVYYCGGYD YSTYTNPFGA GTTLTVL
(SEQ ID NO: 131)
DVQLVESGGG VVRPGESLRL SCEASGFTFS SNAMSWVRQA
PGKGLEWVAG ISSGSDTYYG DSVKGRLTIS RDNSKNILYL
QMNSLTAEDT AVYYCARETW NHLFDYWGLG TLVTVSSAKT
TAPSVYPLAP VCGDTTGSSV TLGCLVKGYF PEPVTLTWNS
GSLSSGVHTF PAVLQSDLYT LSSSVTVTSS TWPSQSITCN
VAHPASSTKV DKKIEPRGPT IKPCPPCKCP GSGSHHHHHH
GLNDIFEAQK IEWHE
(SEQ ID NO: 132)
ALTQPASVSA SPGETVEITC SGGSDSSYYY GWYQQKSPGS
APVTVIYSDN KRPSNIPSRF SGSASGSTAT LTITGVRVED
EAVYYCGGYD YSTYTNPFGA GTTLTVLRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC

In some embodiments, the anti-SIRP antibody comprises a heavy chain that comprises SEQ ID NO: 117 and a light chain that comprises SEQ ID NO: 118. In some embodiments, the anti-SIRP antibody comprises a heavy chain that comprises SEQ ID NO: 119 and a light chain that comprises SEQ ID NO: 118. In some embodiments, the anti-SIRP antibody comprises a heavy chain that comprises SEQ ID NO: 120 and a light chain that comprises SEQ ID NO: 121. In some embodiments, the anti-SIRP antibody comprises a heavy chain that comprises SEQ ID NO: 122 and a light chain that comprises SEQ ID NO: 121. The amino acid sequences of SEQ ID NOs: 117-122 are provided below.

(SEQ ID NO: 117)
DVQLVESGGG VVRPGESLRL SCAASGFTFS SNAMSWVRQA
PGKGLEWLAG ISAGGSDTYY PASVKGRFTI SRDNSKNTLY
LQMNTLTAED TAVYYCARET WNHLFDYWGL GTLVTVSSAK
TTPPSVYPLA PGSAAQTNSM VTLGCLVKGY FPEPVTVTWN
SGSLSSGVHT FPAVLQSDLY TLSSSVTVPS STWPSETVTC
NVAHPASSTK VDKKIVPRDC GCKPCICTVP EVSSVFIFPP
KPKDVLTITL TPKVTCVVVD ISKDDPEVQF SWFVDDVEVH
TAQTQPREEQ FASTFRSVSE LPIMHQDWLN GKEFKCRVNS
AAFPAPIEKT ISKTKGRPKA PQVYTIPPPK EQMAKDKVSL
TCMITDFFPE DITVEWQWNG QPAENYKNTQ PIMDTDGSYF
IYSKLNVQKS NWEAGNTFTC SVLHEGLHNH HTEKSLSHSP G
(SEQ ID NO: 118)
ALTQPASVSA NPGETVKIAC SGGDYYSYYY GWYQQKAPGS
ALVTVIYSDD KRPSDIPSRF SGSASGSTAT LTITGVRAED
EAVYYCGGYD YSTYANAFGA GTTLTVLGQP KSSPSVTLFP
PSSEELETNK ATLVCTITDF YPGVVTVDWK VDGTPVTQGM
ETTQPSKQSN NKYMASSYLT LTARAWERHS SYSCQVTHEG
HTVEKSLSRA DCS
(SEQ ID NO: 119)
DVQLVESGGG VVRPGESLRL SCAASGFTFS SNAMSWVRQA
PGKGLEWLAG ISAGGSDTYY PASVKGRFTI SRDNSKNTLY
LQMNTLTAED TAVYYCARET WNHLFDYWGL GTLVTVSSAK
TTAPSVYPLA PVCGDTTGSS VTLGCLVKGY FPEPVTLTWN
SGSLSSGVHT FPAVLQSDLY TLSSSVTVTS STWPSQSITC
NVAHPASSTK VDKKIEPRGP TIKPCPPCKC PAPNLLGGPS
VFIFPPKIKD VLMISLSPIV TCVVVDVSED DPDVQISWFV
NNVEVHTAQT QTHREDYNST LRVVSALPIQ HQDWMSGKEF
KCKVNNKDLP APIERTISKP KGSVRAPQVY VLPPPEEEMT
KKQVTLTCMV TDFMPEDIYV EWTNNGKTEL NYKNTEPVLD
SDGSYFMYSK LRVEKKNWVE RNSYSCSVVH EGLHNHHTTK
SFSRTPG
(SEQ ID NO: 120)
DVQLVESGGG VVRPGESLRL SCAASGFTFS SYAMSWVRQA
PGKGLEWLAG ISAGGSDTYY IDSVKGRFTI SRDNPKNSLY
LQMSSLTAED TAVYYCARET WNHLFDYWGL GTLVTVSSAK
TTPPSVYPLA PGSAAQTNSM VTLGCLVKGY FPEPVTVTWN
SGSLSSGVHT FPAVLQSDLY TLSSSVTVPS STWPSETVTC
NVAHPASSTK VDKKIVPRDC GCKPCICTVP EVSSVFIFPP
KPKDVLTITL TPKVTCVVVD ISKDDPEVQF SWFVDDVEVH
TAQTQPREEQ FASTFRSVSE LPIMHQDWLN GKEFKCRVNS
AAFPAPIEKT ISKTKGRPKA PQVYTIPPPK EQMAKDKVSL
TCMITDFFPE DITVEWQWNG QPAENYKNTQ PIMDTDGSYF
IYSKLNVQKS NWEAGNTFTC SVLHEGLHNH HTEKSLSHS PG
(SEQ ID NO: 121)
ALTQPASVSA NPGETVKITC SGGDYYSTYY AWYQQKSPGS
APVTVIHSDD KRPSDIPSRF SGSASGSAAT LIITGVRVED
EAVYYCGGYD GRTYINTFGA GTTLTVLGQP KSSPSVTLFP
PSSEELETNK ATLVCTITDF YPGVVTVDWK VDGTPVTQGM
ETTQPSKQSN NKYMASSYLT LTARAWERHS SYSCQVTHEG
HTVEKSLSRA DCS
(SEQ ID NO: 122
DVQLVESGGG VVRPGESLRL SCAASGFTFS SYAMSWVRQA
PGKGLEWLAG ISAGGSDTYY IDSVKGRFTI SRDNPKNSLY
LQMSSLTAED TAVYYCARET WNHLFDYWGL GTLVTVSSAK
TTAPSVYPLA PVCGDTTGSS VTLGCLVKGY FPEPVTLTWN
SGSLSSGVHT FPAVLQSDLY TLSSSVTVTS STWPSQSITC
NVAHPASSTK VDKKIEPRGP TIKPCPPCKC PAPNLLGGPS
VFIFPPKIKD VLMISLSPIV TCVVVDVSED DPDVQISWFV
NNVEVHTAQT QTHREDYNST LRVVSALPIQ HQDWMSGKEF
KCKVNNKDLP APIERTISKP KGSVRAPQVY VLPPPEEEMT
KKQVTLTCMV TDFMPEDIYV EWTNNGKTEL NYKNTEPVLD
SDGSYFMYSK LRVEKKNWVE RNSYSCSVVH EGLHNHHTTK
SFSRTPG

In some embodiments, the anti-SIRP multimer comprises an anti-SIRP antibody comprising a VH that comprises SEQ ID NO: 115 and a VL that comprises SEQ ID NO: 116. In some embodiments, the anti-SIRP multimer (e.g., the anti-SIRP antibody) is added to the plasma sample (e.g., a plasma sample obtained by a subject who has received treatment with drug) to achieve about any one of a 1-fold, 1.5-fold, 2-fold, 2.5-fold, 3-fold, 3.5-fold, 4-fold, 4.5-fold, 5-fold 5.5-fold, 6-fold, 6.5-fold, 7-fold, 7.5-fold, 8-fold, 8.5-fold, 9-fold, 9.5-fold, or 10-fold molar excess of the anti-SIRP multimer relative to the amount of drug in the plasma.

In some embodiments, the anti-SIRP multimer comprises an anti-SIRP antibody comprising a heavy chain that comprises SEQ ID NO: 119 and a light chain that comprises SEQ ID NO: 118. In some embodiments, the anti-SIRP multimer (e.g., the anti-SIRP antibody) is added to the plasma sample (e.g., a plasma sample obtained by a subject who has received treatment with drug) to achieve about any one of a 1-fold, 1.5-fold, 2-fold, 2.5-fold, 3-fold, 3.5-fold, 4-fold, 4.5-fold, 5-fold 5.5-fold, 6-fold, 6.5-fold, 7-fold, 7.5-fold, 8-fold, 8.5-fold, 9-fold, 9.5-fold, or 10-fold molar excess of the anti-SIRP multimer relative to the amount of drug in the plasma.

In some embodiments, the anti-SIRP multimer comprises an anti-SIRP antibody comprising a heavy chain that comprises SEQ ID NO: 117 and a light chain that comprises SEQ ID NO: 118. In some embodiments, the anti-SIRP multimer (e.g., the anti-SIRP antibody) is added to the plasma sample (e.g., a plasma sample obtained by a subject who has received treatment with drug) to achieve about any one of a 1-fold, 1.5-fold, 2-fold, 2.5-fold, 3-fold, 3.5-fold, 4-fold, 4.5-fold, 5-fold 5.5-fold, 6-fold, 6.5-fold, 7-fold, 7.5-fold, 8-fold, 8.5-fold, 9-fold, 9.5-fold, or 10-fold molar excess of the anti-SIRP multimer relative to the amount of drug in the plasma.

In some embodiments, the anti-SIRP multimer comprises an anti-SIRP antibody comprising a heavy chain that comprises SEQ ID NO: 120 and a light chain that comprises SEQ ID NO: 121. In some embodiments, the anti-SIRP multimer (e.g., the anti-SIRP antibody) is added to the plasma sample (e.g., a plasma sample obtained by a subject who has received treatment with drug) to achieve about any one of a 1-fold, 1.5-fold, 2-fold, 2.5-fold, 3-fold, 3.5-fold, 4-fold, 4.5-fold, 5-fold 5.5-fold, 6-fold, 6.5-fold, 7-fold, 7.5-fold, 8-fold, 8.5-fold, 9-fold, 9.5-fold, or 10-fold molar excess of the anti-SIRP multimer relative to the amount of drug in the plasma.

Other exemplary anti-SIRP antibodies (e.g., anti-SIRPα antibodies, anti-SIRPβ antibodies, and/or anti-SIRPγ antibodies that cross-react with SIRPα) or drug-binding fragments thereof that can be included in the SIRP multimers used in the methods described herein are known in the art. Further details regarding such antibodies are provided in, e.g., WO 2018/057669; US-2018-0105600-A1; US20180312587; WO2018107058; WO2019023347; US20180037652; WO2018210795; WO2017178653; WO2018149938; WO2017068164; and WO2016063233, the contents of which are incorporated herein by reference in their entireties. In some embodiments, an anti-SIRP antibody disclosed in one of the aforementioned references comprises a murine Fc domain (or a portion thereof). In some embodiments, the murine Fc domain comprises an amino acid sequence set forth in any one of SEQ ID NOs: 81-83.

In some embodiments, the anti-SIRP multimer is a homomultimer that comprises identical anti-SIRP antibodies (e.g., identical anti-SIRPα antibodies, anti-SIRPα variant antibodies, anti-SIRPβ variant antibodies, anti-SIRPγ antibodies, anti-SIRPγ variant antibodies, or antibodies capable of cross reacting with one or more of a SIRPα, a SIRPα variant, a SIRPβ variant, a SIRPγ, and/or a SIRPγ variant) or drug binding fragments thereof. In some embodiments, the anti-SIRP multimer is a homomultimer that comprises a monospecific bivalent anti-SIRP antibody. In some embodiments, the SIRP multimer is a heteromultimer that comprises at least two different anti-SIRP antibodies or drug-binding fragments thereof in any combination. In some embodiments, the anti-SIRP multimer is a heteromultimer that comprises a multispecific anti-SIRP antibody (i.e., a multispecific anti-SIRP antibody that comprises a first VH/VL pair and a second VH/VL pair, wherein the first and second VH/VL pairs comprise different amino acid sequences, and wherein the first and second VH/VL pairs each bind the CD47-binding moiety of the drug.)

In some embodiments, the anti-SIRP antibody or drug binding fragment thereof comprises a comprises a multimerization domain, e.g., including, but not limited to the exemplary multimerization domains discussed above for CD47 polypeptide monomers and SIRP polypeptide monomers.

In some embodiments, the anti-SIRP antibody or drug binding fragment thereof comprises (e.g., further comprises) an epitope tag. In some embodiments, the epitope tag facilitates multimerization of the anti-SIRP antibodies (or drug binding fragments thereof). In some embodiments, the tag facilitates the immobilization of the anti-SIRP antibodies (or drug binding fragments thereof) to a solid support (e.g. beads, glass sides, etc.). Exemplary epitope tags include, but are not limited to SEQ ID NOs: 7-32 described elsewhere herein.

In some embodiments, the anti-SIRP antibody (or drug binding fragment thereof) comprises (e.g., is attached to) a ligand. In some embodiments, the ligand is biotin. In some embodiments, the anti-SIRP antibody (or drug binding fragment thereof, e.g., a Fab or F(ab′)2) comprises HHHHHHGLNDIFEAQKIEWHE (SEQ ID NO: 135) or GSGSHHHHHHGLNDIFEAQKIEWHE (SEQ ID NO: 126). GLNDIFEAQKIEWHE (SEQ. ID NO: 8) also known as AVITAG™, is specifically biotinylated by the E. coli biotin ligase BirA, In some embodiments, the Fab or F(ab′)2 comprises SEQ ID NOs: 131 and 132. In some embodiments, the anti-SIRP multimer (e.g., the Fab or F(ab)2 comprising SEQ ID NOs: 131 and 132) is added to the plasma sample (e.g., a plasma sample obtained by a subject who has received treatment with drug) to achieve about any one of a 1-fold, 1.5-fold, 2-fold, 2.5-fold, 3-fold, 3.5-fold, 4-fold, 4.5-fold, 5-fold 5.5-fold, 6-fold, 6.5-fold, 7-fold, 7.5-fold, 8-fold, 8.5-fold, 9-fold, 9.5-fold, or 10-fold molar excess of the anti-SIRP multimer relative to the amount of drug in the plasma.

In some embodiments, the anti-SIRP antibodies (or drug binding fragments thereof) of an anti-SIRP multimer are linked, e.g., via peptide bond, to form a concatenated chain of anti-SIRP antibodies (or drug binding fragments thereof). In some embodiments, the anti-SIRP antibodies (or drug binding fragments thereof) in a SIRP multimer are linked via linker peptide. Exemplary linker peptides comprise, but are not limited to, those set forth in SEQ ID NO: 85-109, 127-130, IEGR (SEQ ID NO: 141), IDGR (SEQ ID NO: 140), and other linkers that find use with CD47 multimers and/or SIRP multimers (see above). In some embodiments, the anti-SIRP antibodies (or drug binding fragments thereof) of an anti-SIRP multimer are linked via linker peptide and at least one spacer. Exemplary peptide spacers include, but are not limited to, SEQ ID NOs: 52-70 and other spacers that find use with CD47 multimers and/or SIRP multimers (see above).

In some embodiments, the anti-SIRP multimer comprises at least one (e.g., between 1 and 100) anti-SIRP antibodies (or drug binding fragments thereof) attached to a solid support. In some embodiments, the anti-SIRP antibody, or the anti-SIRP antibodies or drug binding fragments thereof, is/are attached to the solid support via covalent bond or via non-covalent capture. In some embodiments, the solid support is a gold nanosphere, a gold nanoshell, a magnetic bead, a silica bead, a dextran polymer, a tube, a slide, a gel column, or microtiter wells. In some embodiments, the solid support comprises or is fabricated from, e.g., glass, cellulose, polyacrylamide, nylon, polystyrene, polyvinyl chloride or polypropylene. In some embodiments, anti-SIRP multimer comprises an anti-SIRP antibody, or more than one anti-SIRP antibodies (or drug binding fragment thereof), and a solid support, wherein the anti-SIRP antibody, or the anti-SIRP antibodies or drug binding fragments thereof, comprise an epitope tag or ligand (e.g., as described above), wherein capture agents are immobilized on the solid support, and wherein the anti-SIRP antibody, or the anti-SIRP antibodies or drug binding fragments thereof, are attached to the solid support via the specific binding of the epitope tags or ligands by the capture agents immobilized on the solid support. In some embodiments, the anti-SIRP antibody, or the anti-SIRP antibodies or drug binding fragments thereof, comprise SEQ ID NO: 111. In some embodiments, the ligand is biotin and the capture agent is streptavidin. In some embodiments, the anti-SIRP multimer (e.g., homomultimer or heteromultimer) comprises a streptavidin or an avidin bound to 2, 3, or 4 biotinylated anti-SIRP antibodies (or drug binding fragments thereof). In some embodiments, the anti-SIRP antibodies (or drug binding fragments thereof) comprise SEQ ID NO: 111. In some embodiments, the anti-SIRP multimer comprises a streptavidin or avidin bound to 2, 3, or 4, F(ab′)2 fragments. In some embodiments, two or more of the F(ab′)2 fragments comprise SEQ ID NO: 131 and SEQ ID NO: 132.

(c) Methods of Making Anti-SIRP Multimers

In some embodiments, an anti-SIRP multimer (e.g., a homomultimer or heteromultimer comprising, e.g., two or more anti-SIRP antibodies (or drug binding fragments thereof), e.g., linked via peptide bond or via linker peptide, is produced by a recombinant host cell. Any of the methods described herein for producing a CD47 multimer or SIRP multimer using recombinant techniques are applicable for the production of an anti-SIRP multimer comprising anti-SIRP antibodies (or antigen binding fragments thereof), as well.

In some embodiments, an anti-SIRP multimer (e.g., homomultimer or heteromultimer) is produced by attaching one or more (e.g., between 1 and 100) anti-SIRP antibodies (or drug binding fragments thereof) to a solid support via non-covalent capture. In some embodiments, the solid support is a gold nanosphere, a gold nanoshell, a magnetic bead, a silica bead, a dextran polymer, a tube, a slide, a gel column, or microtiter wells. In some embodiments, the anti-SIRP antibody, or the anti-SIRP antibodies (or drug binding fragments thereof), each comprise a ligand, and capture agents are immobilized on the solid support, and the anti-SIRP multimer is produced by attaching the anti-SIRP antibody, or the anti-SIRP antibodies (or drug binding fragments thereof), to the solid support via the specific binding of the ligands by the capture agents immobilized on the solid support. In some embodiments, the ligand is biotin and the capture agent is streptavidin. In some embodiments, the anti-SIRP antibody, or the anti-SIRP antibodies (or drug binding fragments thereof) comprise SEQ ID NO: 111 or SEQ ID NO: 126.

Any of the methods described elsewhere herein for attaching CD47 polypeptide monomers to a solid support to produce a CD47 multimer are applicable for attaching anti-SIRP antibodies (or drug-binding fragments thereof) to a solid support to produce an anti-SIRP multimer, as well. For example, in some embodiments, an anti-SIRP multimer is prepared by culturing host cell comprising a nucleic acid encoding an anti-SIRP antibody (or drug-binding fragment thereof) that comprises SEQ ID NO: 111 or SEQ ID NO: 126 under appropriate conditions to cause expression of the anti-SIRP antibody (or drug-binding fragment thereof) and recovering the anti-SIRP antibody (or drug-binding fragment thereof). The anti-SIRP antibody (or drug-binding fragment thereof), which comprises biotin-acceptor peptide amino acid sequence, is biotinylated using E. coli biotin ligase (BirA). In some embodiments, the anti-SIRP multimer is produced by attaching biotinylated anti-SIRP antibody or antibodies (or drug-binding fragments thereof) to a solid support onto which streptavidin or avidin molecules have been immobilized. In some embodiments, provided is an anti-SIRP multimer (e.g., homomultimer or heteromultimer) that comprises one or more (e.g., between 1 and 100) anti-SIRP antibodies (or drug-binding fragments thereof) attached to, e.g., a streptavidin- or avidin-conjugated solid support. Exemplary streptavidin- or avidin-conjugated solid supports are described in further detail elsewhere herein. In some embodiments, provided is an anti-SIRP multimer (e.g., homomultimer or heteromultimer) comprises a streptavidin or an avidin bound to 2, 3, or 4 biotinylated anti-SIRP antibodies (or drug-binding fragments thereof).

In some embodiments, the anti-SIRP multimer is produced by linking one or more anti-SIRP antibodies (or drug binding fragments thereof) to, e.g., a solid support, or, e.g., to each other, using bifunctional crosslinkers (e.g., such as those described elsewhere herein).

The anti-SIRP multimers thus produced are then used in a method provided herein to prevent interference by a drug that binds CD47 in serological assays.

### V. Exemplary Drugs

The methods provided herein reduce (or, in some embodiments, eliminate) interference in serological assays caused by the presence of a drug comprising (i) an antibody Fc region and (ii) a moiety that binds to human CD47 in a sample comprising plasma or RBCs/platelets obtained from a subject who has received treatment with the drug. In some embodiments, the drug comprises an IgG Fc region, such as a human IgG Fc region, e.g., an IgG1, IgG2, or an IgG4 Fc region. In some embodiments, the drug comprises a modified Fc region (such as a modified IgG Fc region) that comprises one or more amino acid substitution(s), deletion(s), insertion(s), N-terminal addition(s) and/or C-terminal addition(s) relative to a wild type human Fc region (e.g., a wild type human IgG Fc region). Exemplary Fc regions are described in WO2017177333; WO2014094122; US2015329616, WO 2017/027422; US 2017/0107270; and U.S. Pat. No. 10,259,859, the contents of which are incorporated herein by reference in their entirety.

In some embodiments, the moiety that binds to human CD47 is a wild type SIRPα that lacks a transmembrane domain (e.g., the extracellular domain of any wild type SIRPα that is capable of binding human CD47). In some embodiments, the moiety that binds to human CD47 is a SIRPα variant that is capable of binding human CD47 and lacks a transmembrane domain. In some embodiments, the SIRPα variant comprises one or more amino acid substitution(s), deletion(s), insertion(s), N-terminal addition(s) and/or C-terminal addition(s) relative to extracellular domain of a wild-type SIRPα. In some embodiments, the SIRPα variant is a SIRPα-d1 domain variant. In some embodiments the affinity of the SIRPα variant for human CD47 is higher than the affinity of a wild type SIRPα for human CD47.

In some embodiments, the moiety that binds to human CD47 is a wild type SIRPγ that lacks a transmembrane domain (e.g., the extracellular domain of any wild type SIRPγ that is capable of binding human CD47). In some embodiments, the moiety that binds to human CD47 is a SIRPγ variant that is capable of binding human CD47 and lacks a transmembrane domain. In some embodiments, the SIRPγ variant comprises one or more amino acid substitution(s), deletion(s), insertion(s), N-terminal addition(s) and/or C-terminal addition(s) relative to extracellular domain of a wild-type SIRPγ. In some embodiments, the SIRPγ variant is a SIRPγ-d1 domain variant. In some embodiments the affinity of the SIRPγ variant for human CD47 is higher than the affinity of a wild type SIRPγ for human CD47.

In some embodiments, the moiety that binds to human CD47 is a SIRPβ variant that is capable of binding human CD47 and lacks a transmembrane domain. In some embodiments, the SIRPβ variant comprises one or more amino acid substitution(s), deletion(s), insertion(s), N-terminal addition(s) and/or C-terminal addition(s) relative to extracellular domain of a wild-type SIRPβ. In some embodiments, the SIRPβ variant is a SIRPβ-d1 domain variant.

Exemplary SIRPα variants, SIRPβ variants, and SIRPγ variants are known in the art and are described in WO 2013/109752; US 2015/0071905; U.S. Pat. No. 9,944,911; WO 2016/023040; WO 2017/027422; US 2017/0107270; U.S. Pat. Nos. 10,259,859; 9,845,345; WO2016187226; US20180155405; WO2017177333; WO2014094122; US2015329616; US20180312563; WO2018176132; WO2018081898; WO2018081897; US20180141986A1; and EP3287470A1, the contents of which are incorporated herein by reference in their entireties.

In some embodiments of any of the methods described above, the drug is an anti-CD47 antibody. In some embodiments, the anti-CD47 antibody is AO-176, CC-90002, Hu5F9-G4 (also referred to as 5F9), SHR-1603, NI-1701, SRF231, TJC4, or IBI188. Details regarding these and other therapeutic anti-CD47 antibodies are provided in WO2018175790A1; US20180142019; US20180171014; US20180057592; US20170283498, U.S. Pat. Nos. 9,518,116; 9,518,117; US20150274826; US20160137733; U.S. Pat. No. 9,221,908; US20140161799; US20160137734; WO2015191861; WO2014093678; WO2014123580; WO2013119714; U.S. Pat. No. 9,045,541; WO2016109415; WO2018183182; WO2018009499; WO2017196793; U.S. Pat. No. 9,663,575; US20140140989; WO2018237168; US20180037652; US20190023784; WO2018095428; EP3411071; WO2019042285; WO2016081423; WO2011076781; WO2012172521; WO2014087248; US20140303354; WO2016156537; US20160289727; US20190062428; US20180201677; U.S. Pat. No. 9,352,037; US20170044258; U.S. Pat. No. 9,650,441; and US20180105591

### VI. Exemplary Serological Assays for Pre-Transfusion Testing

Pre-transfusion testing is performed to ensure that the blood product intended for transfusion is compatible with the blood of the subject (i.e., the recipient of the transfusion). Pre-transfusion testing encompasses the serological assays that are used to confirm ABO compatibility between donor blood and recipient blood, as well as those that are used to detect most clinically significant RBC/platelet alloantibodies that react with antigens on donor RBCs and/or donor platelets (ref. Technical Manual, 18th ed, AABB, Bethesda, Md., 2014). Other exemplary blood group antigens for which serological assays are performed to determine donor/recipient transfusion compatibility include, without limitation, e.g., Kell blood group antigens, Duffy blood group antigens, Knops blood group antigens, Cartwright blood group antigens, Scianna blood group antigens, Indian blood group antigens, Rhesus blood group antigens, Dombrock blood group antigens, Landsteiner-Wiener blood group antigens, and VEL blood group antigens. The methods provided herein reduce or prevent drug interference (e.g., interference by a drug comprising (i) an antibody Fc region and (ii) a moiety that binds to human CD47) in a number of serological assays known in the art. Exemplary serological assays in which the methods can be used include (but are not limited to) those described in further detail below.

Typically, serological assays are performed using samples comprising, e.g., non-hemolyzed blood, plasma (e.g., a plasma sample that has been anticoagulated in EDTA), clotted blood, or serum from a subject who is in need of the transfusion (e.g., a subject who has received treatment with a drug comprising (i) an antibody Fc region and (ii) a moiety that binds to human CD47. In general, the subject's ABO group and Rh type are determined first. Next, an antibody screening method is used to detect any clinically significant unexpected non-ABO blood group antibodies that may be present in the subject's plasma. If the screening test reveals the presence of such an antibody, the specificity of that antibody is determined using an antibody identification panel. After the specificity of the antibody is identified, donor units of the appropriate ABO group and Rh type are screened for the corresponding antigen. Units that are negative for that antigen are crossmatched with the subject who is in need of the transfusion to ensure compatibility.

Serological assays can be performed in a tube, on a slide, on a gel column or in microtiter well plates, and hemolysis and agglutination are signals that indicate a positive (incompatible) test result. Agglutination, a reaction reflecting linkage of adjacent RBCs that are coated with antibody, can be scored macroscopically and/or microscopically and on scale from 0-4+ in the most commonly used tube methods. A score of zero indicates no reactivity and is characterized by smooth and easily dispersed cells. A score of 4+ indicates strong reactivity and is characterized by one solid agglutinate that is not easily dispersed. Scores of 1+, 2+, or 3+ indicate intermediate levels of reactivity, characterized by gradually increasing size of agglutinates with higher scores. Similar principles of agglutination scoring can be applied when the serological tests are conducted using gel columns with anti IgG antibody in the column (gel card) or microtiter well plates with bound red blood cell antigens (solid phase). Various techniques are currently available for the detection of antibody-RBC antigen interaction with varying sensitivities. In some embodiments, serological assays are performed manually. In some embodiments, serological assays are performed via automated machine.

For example, immediate-spin (IS) (also known as “immediate spin crossmatch”) is an assay that entails mixing, e.g., reagent plasma/antisera (i.e., plasma containing antibodies against a known RBC and/or platelet surface antigen) and the subject's blood cells, immediately centrifuging the mixture for about 15-30 seconds at room temperature or at 37° C., and visually examining the tube for direct agglutination. Direct agglutination indicates that there is a strong interaction between an antibody in the plasma and an RBC surface antigen. Alternatively, the subject's plasma and reagent RBC (i.e., RBC that are known to express a particular cell surface antigen, or group of cell surface antigens) and/or regent platelets (i.e., platelets that are known to express a particular cell surface antigen, or group of cell surface antigens) can be mixed, centrifuged, and assessed visually for direct agglutination.

Anti-human globulins (AHGs) are used to detect antibody-bound RBC that do not produce direct agglutination. AHG are secondary anti-human globulin antibodies that have been produced in another species. AHG reagents can be specific for a single class of human Ig (such as IgG), or polyspecific, i.e., capable of binding to multiple human Ig classes (e.g., IgG, IgM, IgA) and to complement. AHG sera may be used in a direct antiglobulin test (DAT) and/or in an indirect antiglobulin test (TAT). The DAT demonstrates in vivo sensitization of red cells and is performed by directly testing a sample of washed patient red cells with AHG. An IAT demonstrates in vitro reactions between red cells and antibodies. In an IAT, serum (or plasma) is incubated with red cells, which are then washed to remove unbound globulins. The presence of agglutination with the addition of AHG indicates antibody binding to a specific red cell antigen. Some methods involve addition of potentiator reagents (enhancement) such as saline, albumin, low ionic strength saline (LISS), or polyethylene glycol (PEG), and the samples are then incubated at 37° C. for 10-60 minutes prior to the AHG test. In some embodiments, the assay is a tube assay. In some embodiments, the assay is a solid phase red cell adherence assay (SPRCA).

ABO typing involves testing the recipient's red blood cells for the presence of A and B antigens using anti-A and anti-B antisera (forward grouping). Testing of the recipient plasma for the presence of anti-A and anti-B using known Type A and Type B red blood cells (reverse grouping) is also part of routine ABO blood group testing.

The Rh (D) type of the transfusion recipient is determined by testing recipient red blood cells with anti-D. ABO grouping is typically tested using immediate spin (IS).

Alloantibodies to antigens that are not present on an individual's red blood cells may develop in anyone who has been exposed to foreign red blood cell antigens through pregnancy or transfusion. To detect antibodies to non-group A or B antigens, a sample of the patient's plasma or serum is tested against selected commercial Type O red blood cells that express the majority of clinically significant antigens, other than A and B.

In cases of positive antibody screening, further serological testing is conducted with an expanded panel of commercial Type O reagent RBCs for the identification of clinically significant antibodies is required. Then, once the specificity of the antibody is known, donor units must be screened for the corresponding antigen to select those units that lack the antigen.

Antigen typing (phenotyping) of the recipient red blood cells may also be performed to determination of which red blood cell antibodies an individual is likely to develop. Serological assay for RBC phenotyping involves mixing recipient cells with commercial reagent anti-sera containing specific antibodies.

An IAT without and with enhancement (e.g. saline, LISS, PEG) is used in antibody detection and antibody identification.

“Crossmatch” refers to a method of confirming compatibility between the patient's blood (plasma) and the donor red blood cells. The crossmatch is meant primarily to detect and prevent ABO incompatibility. A serological crossmatch assay (either IS crossmatch or AHG phase crossmatch) involves the direct mixing of donor red blood cells with recipient plasma and scores for hemolysis and agglutination following immediate-spin method or AHG test.

In some embodiments, the serological assay is performed using a gel card. The principle of the gel card test is based on the gel technique described in Lapierre, et al. (1990) “The gel test: a new way to detect red cells antigen-antibody reactions.” Transfusion, 30: 109-113 for detecting red blood cell agglutination reactions. The plastic cards are composed of multiple microtubes. Each microtube comprises an incubator chamber on top of a column. Each microtube in the card is prefilled with a buffered gel solution containing a specific antibody against an RBC- or platelet-surface antigen. The agglutination occurs if a subject's RBC or platelets react with the corresponding antibodies present in the gel solution. Alternatively, the microtube is filled with a subject's serum, and agglutination occurs if reagent RBCs or reagent platelets react with antibodies present in the patient's serum or plasma. The gel column acts as a filter that traps agglutinated red blood cells as they pass through the gel column during the centrifugation of the card. The gel column separates agglutinated red blood cells from non-agglutinated red blood cells based on size. Any agglutinated red blood cells are captured at the top of or along the gel column, and non-agglutinated red blood cells reach the bottom of the microtube forming a pellet.

In some embodiments, the serological assay is a solid phase assay (e.g., wherein a multimer that mitigates/reduces interference of drugs that bind CD47 described herein, the subject's RBCs, reagent RBCs, or antibodies against known RBC- and/or platelet-surface antigens are immobilized on a solid support). In some embodiments, the serological assay is performed manually. In some embodiments, the serological assay is performed using an automated system. In some embodiments, the automated system is a multiplexed high-throughput system that permits the simultaneous analysis of a plurality of RBC, platelet, serum, or plasma samples obtained from different subjects. In some embodiments, the automated system is a multiplexed high-throughput system that permits the simultaneous analysis of a variety of serological assays using RBC, platelet, serum, or plasma samples obtained from a single subject. Typically, automated blood analysis systems are microprocessor-controlled instruments. Exemplary automated blood analysis systems include, e.g., Immucor's NEO® system (i.e., a solid-phase platform) and Diagast's QWALYS® 3 system. Qwalys® is fully automated erythrocyte-magnetized technology (EMT) system for ABO/D grouping, Rh phenotyping, K typing, and antibody screening (ABS).

The specification is considered to be sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes.

Each embodiment herein described may be combined with any other embodiment or embodiments unless clearly indicated to the contrary. In particular, any feature or embodiment indicated as being preferred or advantageous may be combined with any other feature or features or embodiment or embodiments indicated as being preferred or advantageous, unless clearly indicated to the contrary.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1: Preparation of an Exemplary CD47 Multimer

### Preparation of CD47 Multimer B

CD47 polypeptide monomers comprising SEQ ID NO: 6 (see below) were transiently expressed in Expi293F cells, harvested, and purified using Ni SEPHAROSE® 6 FAST FLOW chromatography resin and polished via size exclusion chromatography.

(SEQ ID NO: 6)
QLLFNKTKSV EFTFSNDTVV IPCFVTNMEA QNTTEVYVKW
KFKGRDIYTF DGALNKSTVP TDFSSAKIEV SQLLKGDASL
KMDKSDAVSH TGNYTCEVTE LTREGETIIE LKYRVVSHHH
HHHGLNDIFE AQKIEWHE

The purified CD47 polypeptide monomers were biotinylated in vitro using a BirA biotinylation kit available from Avidity LLC. The in vitro biotinylation reaction mixture was then purified via size exclusion chromatography to separate biotinylated CD47 polypeptide monomers from excess biotin present in the mixture.

The biotinylated CD47 polypeptide monomers were then incubated with streptavidin protein at a molar ratio of 4.5:1 biotinylated CD47 polypeptide monomer: streptavidin overnight at 4° C. with gentle agitation. The reaction mixture was then purified via size exclusion chromatography to separate CD47 multimer (i.e., tetramerized biotinylated CD47 polypeptide monomers bound to streptavidin) from other species in the mixture. The CD47 multimer (i.e., tetramer) was confirmed via size exclusion chromatography against a gel filtration standard.

### Example 2: Preparation of Exemplary Anti-SIRP Multimers

### Generation of Anti-SIRP Octamer (Anti-SIRP Multimer C)

An anti-SIRP F(ab′)2 comprising a VH-CH1 sequence comprising SEQ ID NO: 131 and a VL-CL sequence comprising SEQ ID NO: 132 was prepared as follows: The C-terminus of SEQ ID NO: 131 comprises SEQ ID NO: 126, which comprises a hexahistidine peptide HHHHHH (SEQ ID NO: 7) and the 15 amino acid tag GLNDIFEAQKIEWHE (SEQ ID NO: 8). GLNDIFEAQKIEWHE (SEQ ID NO: 8), also known as AVITAG™. SEQ ID NO: 8 is specifically biotinylated by the E coli biotin ligase BirA.

(SEQ ID NO: 131)
DVQLVESGGG VVRPGESLRL SCEASGFTFS SNAMSWVRQA
PGKGLEWVAG ISSGSDTYYG DSVKGRLTIS RDNSKNILYL
QMNSLTAEDT AVYYCARETW NHLFDYWGLG TLVTVSSAKT
TAPSVYPLAP VCGDTTGSSV TLGCLVKGYF PEPVTLTWNS
GSLSSGVHTF PAVLQSDLYT LSSSVTVTSS TWPSQSITCN
VAHPASSTKV DKKIEPRGPT IKPCPPCKCP GSGSHHHHHH
GLNDIFEAQK IEWHE
(SEQ ID NO: 132)
ALTQPASVSA SPGETVEITC SGGSDSSYYY GWYQQKSPGS
APVTVIYSDN KRPSNIPSRF SGSASGSTAT LTITGVRVED
EAVYYCGGYD YSTYTNPFGA GTTLTVLRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC

SEQ ID NOs: 131 and 132 were transiently expressed in Expi293 cells. Transfection supernatant was purified using Ni SEPHAROSE® 6 FAST FLOW chromatography resin and polished via size exclusion chromatography. The purified anti-SIRP (Fab′)2 was biotinylated in vitro using a BirA biotinylation kit available from Avidity LLC. The in vitro biotinylation reaction mixture was then purified via size exclusion chromatography to separate biotinylated anti-SIRP F(ab′)2 from excess biotin present in the mixture.

The biotinylated anti-SIRP F(ab′)2 was then incubated with streptavidin protein at a molar ratio of 4.5:1 biotinylated anti-SIRP F(ab′)2: streptavidin overnight at 4° C. with gentle agitation. The reaction mixture was then purified via size exclusion chromatography to separate anti-SIRP F(ab′)2 multimer (i.e., tetramerized biotinylated anti-SIRP F(ab′)2s bound to streptavidin) from other species in the mixture. The anti-SIRP F(ab′)2 multimer (i.e., octamer) was confirmed via size exclusion chromatography against a gel filtration standard.

### Generation of Anti-SIRP Multimer D

An anti-SIRP antibody comprising a VH that comprises SEQ ID NO: 115, a VL that comprises SEQ ID NO: 116, and a murine Fc domain was transiently expressed in Expi293 cells. (SEQ ID NOs: 115 and 116 are provided below.) The anti-SIRP antibody was purified from the cell culture supernatant via MabSelect LX and dialyzed into lx phosphate buffered saline.

(SEQ ID NO: 115)
DVQLVESGGG VVRPGESLRL SCAASGFTFS SNAMSWVRQA
PGKGLEWLAG ISAGGSDTYY PASVKGRFTI SRDNSKNTLY
LQMNTLTAED TAVYYCARET WNHLFDYWGL GTLVTVSS
(SEQ ID NO: 116)
ALTQPASVSA NPGETVKIAC SGGDYYSYYY GWYQQKAPGS
ALVTVIYSDD KRPSDIPSRF SGSASGSTAT LTITGVRAED
EAVYYCGGYD YSTYANAFGA GTTLTVL

### Example 3: Mitigating the Interference of Drugs that Comprise (i) an Antibody Fc Region and (ii) a Moiety that Binds to Human CD47 in Routine Serological Tests Using CD47 Multimer B, Anti-SIRP Multimer C, or Anti-SIRP Multimer D

Experiments were performed to assess whether CD47 multimer B, anti-SIRP multimer C, or anti-SIRP multimer D mitigate the interference of Drug A in an Indirect Antiglobulin Test (IAT) using the tube method. Drug A is an exemplary CD47-binding drug comprising a SIRPα variant (i.e., a CD47-binding domain derived from human SIRPα) and an Fc variant derived from the Fc region of human immunoglobulin IgG1, and Drug A has previously been shown to interfere with routine serological assays (see Kim et al. (2020) Transfusion. 1-9).

To prepare donor red blood cells (RBC) and donor plasma, HemeQC whole blood samples from donors of different blood types (commercially available from Bio-Rad) were spun at 1000×g for 10 minutes at room temperature to separate plasma from red blood cells. The pelleted red blood cells were then resuspended to 3.4% in MLB2 LISS (low ionic strength solution).

To perform the assay, 100 μL of Drug A at 130 nM (10 μg/mL) was co-incubated with CD47 multimer B, anti-SIRP multimer C, anti-STRP multimer D, or CD47 polypeptide monomer (SEQ ID NO: 6) at 1:2 dilution starting at 20× the molar concentration of Drug A for 10 minutes at room temperature (RT) in each donor plasma. 100 μL of each titration condition was then added to capture wells containing 50 μL of 3.4% RBC from the respective donor and incubated at 37° C. for 45 minutes. The wells were then washed and spun at 400×g for 5 minutes at room temperature three times with PBS. Two drops of AHG anti-IgG (i.e., anti-human globulin IgG) were added to each well and spun at 800×g for 30 seconds. The reaction mixtures were agitated gently to the pelleted cells prior to image capture. Capture wells containing PBS served as negative controls for the assay, and wells containing Drug A alone served as positive controls.

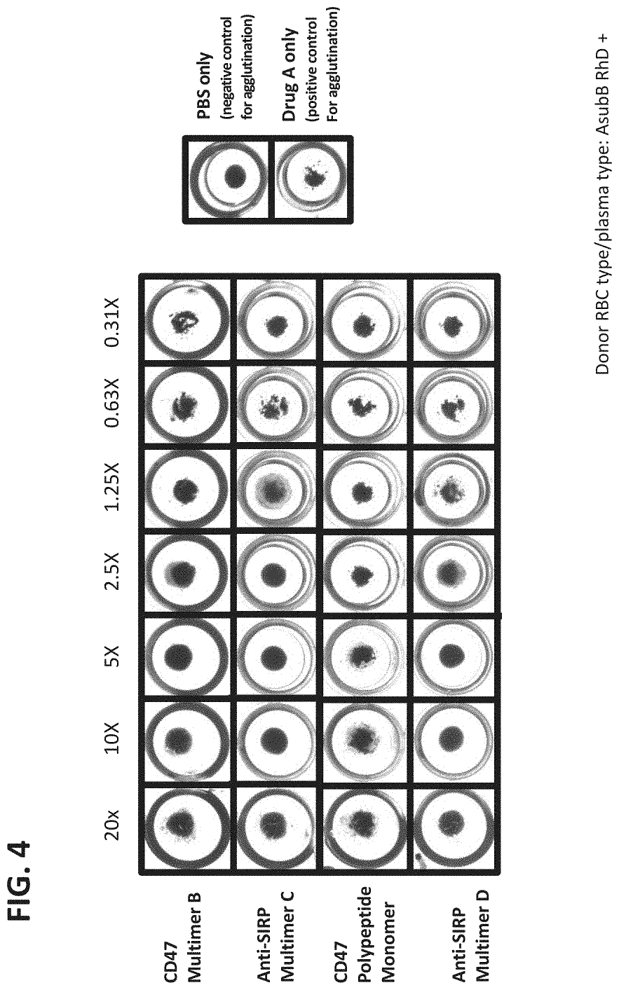

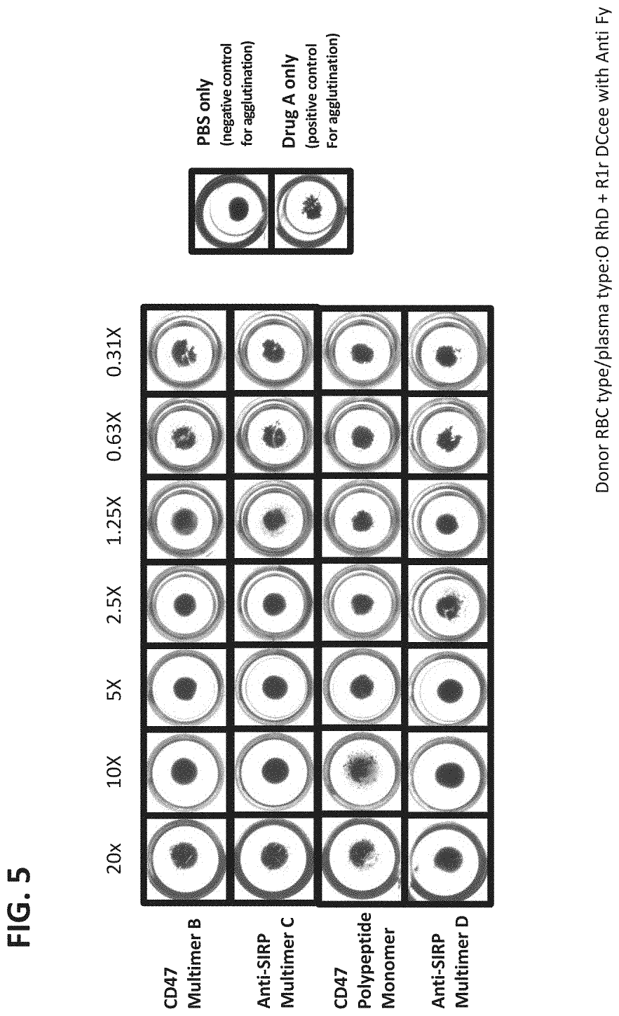

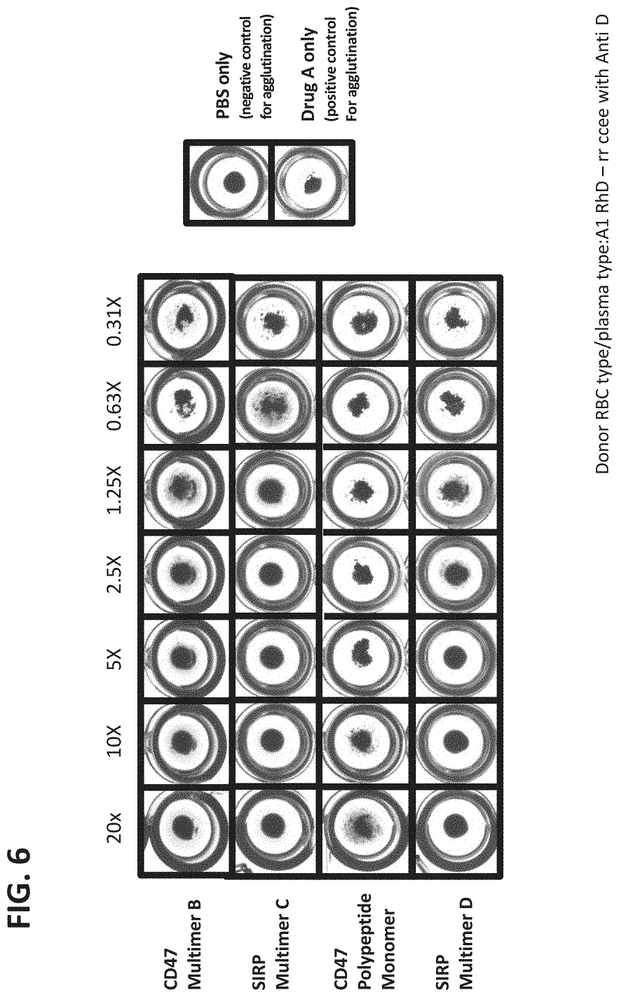

FIG. 4 shows the results of an IAT using the tube assay using a blood sample from a donor with blood type/plasma type AsubB RhD+. FIG. 5 shows the results of an IAT using the tube assay using a blood sample from a donor with RBC type/plasma type O RhD+Rlr DCcee with Anti Fy. FIG. 6 shows the results of an IAT using the tube assay using a blood sample from a donor with blood type/plasma type A1 RhD-rr ccee with Anti D. Different molar titrations (20×, 10×, 5×, 2.5×, 1.25×, 0.63×, 0.31×) of CD47 multimer B, anti-STRP multimer C, anti-SIRP multimer D, and CD47 monomer, i.e., as compared to Drug A, were tested.

Agglutination was observed in the IAT assays with CD47 polypeptide monomer (see FIGS. 4-6) across all molar titrations. By contrast, no agglutination was observed in the IAT assays with addition of at least 5× molar ratio of CD47 multimer B, at 2.5× molar ratio of anti-STRP multimer C, and at 2.5× molar ratio of anti-STRP multimer D. CD47 multimer B, anti-STRP multimer C, and anti-STRP multimer D demonstrated the ability to prevent Drug A from interfering with blood typing when using AHG anti-IgG reagents in a tube assay.

Subsequent experiments were performed to assess whether CD47 multimer B, anti-SIRP multimer C, or anti-STRP multimer D mitigate the interference of Drug A in a solid phase red cell adherence assay (SPRCA).

Briefly, 50 μL of Drug A at 130 nM (10 μg/mL) was co-incubated with CD47 multimer B, anti-STRP multimer C, or anti-STRP multimer D at 1:2 dilution starting at 20× the molar concentration of Drug A for 10 min at room temperature (RT) in PBS. 50 μL of each titration condition was then added to Capture R wells, which are coated with red blood cell membranes prepared from a pool suspension of equal proportions of red cells from two Group O donors. Next, 2 drops of capture LISS reagent was added to each well and the wells were incubated at 37° C. for 45 minutes. The wells were then washed 6× times with PBS. Following the washes, 1 drop of Capture-R Ready Indicator Red cells were then added to each well. The wells were spun at 450×g for 1 minute, and images were captured afterward.

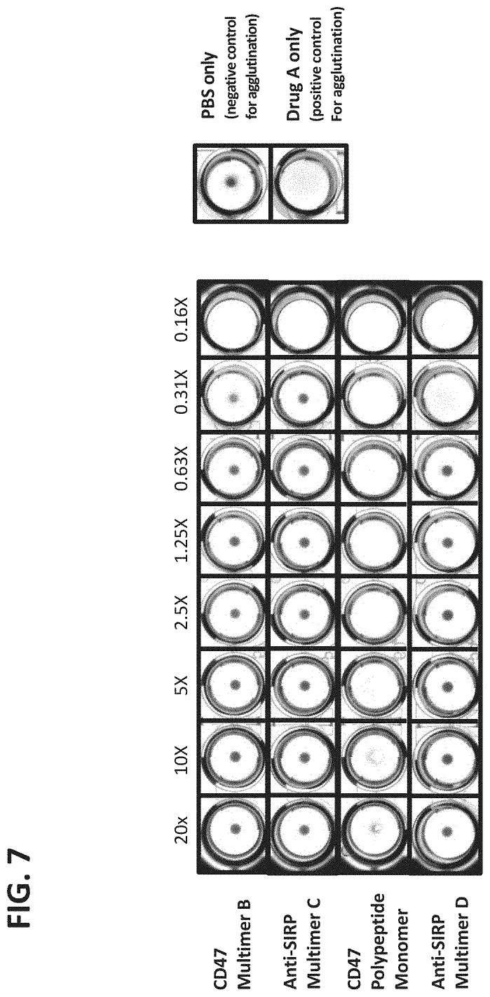

Agglutination was observed in the SPRCA assays with CD47 polypeptide monomer (see FIG. 7). By contrast, no interference by Drug A was observed in the SPRCA assays with addition of at least 0.31× molar ratio of CD47 multimer B, 0.31× molar ratio of anti-STRP multimer C, or 0.63× molar ratio of anti-STRP multimer D. CD47 multimer B, anti-STRP multimer C, and anti-STRP multimer D demonstrated the ability to prevent Drug A from interfering with blood typing when using AHG anti-IgG reagents in a solid phase red cell adherence assay.

### Example 4: Preparation of Exemplary Anti-SIRP Multimer E and Anti-SIRP Multimer F

Anti-STRP multimer E comprises a heavy chain that comprises SEQ ID NO: 117 and a light chain that comprises SEQ ID NO: 118. Anti-STRP multimer F comprises a heavy chain that comprises SEQ ID NO: 120 and a light chain that comprises SEQ ID NO: 121.

(SEQ ID NO: 117)
DVQLVESGGG VVRPGESLRL SCAASGFTFS SNAMSWVRQA
PGKGLEWLAG ISAGGSDTYY PASVKGRFTI SRDNSKNTLY
LQMNTLTAED TAVYYCARET WNHLFDYWGL GTLVTVSSAK
TTPPSVYPLA PGSAAQTNSM VTLGCLVKGY FPEPVTVTWN
SGSLSSGVHT FPAVLQSDLY TLSSSVTVPS STWPSETVTC
NVAHPASSTK VDKKIVPRDC GCKPCICTVP EVSSVFIFPP
KPKDVLTITL TPKVTCVVVD ISKDDPEVQF SWFVDDVEVH
TAQTQPREEQ FASTFRSVSE LPIMHQDWLN GKEFKCRVNS
AAFPAPIEKT ISKTKGRPKA PQVYTIPPPK EQMAKDKVSL
TCMITDFFPE DITVEWQWNG QPAENYKNTQ PIMDTDGSYF
IYSKLNVQKS NWEAGNTFTC SVLHEGLHNH HTEKSLSHSP G
(SEQ ID NO: 118)
ALTQPASVSA NPGETVKIAC SGGDYYSYYY GWYQQKAPGS
ALVTVIYSDD KRPSDIPSRF SGSASGSTAT LTITGVRAED
EAVYYCGGYD YSTYANAFGA GTTLTVLGQP KSSPSVTLFP
PSSEELETNK ATLVCTITDF YPGVVTVDWK VDGTPVTQGM
ETTQPSKQSN NKYMASSYLT LTARAWERHS SYSCQVTHEG
HTVEKSLSRA DCS
(SEQ ID NO: 120)
DVQLVESGGG VVRPGESLRL SCAASGFTFS SYAMSWVRQA
PGKGLEWLAG ISAGGSDTYY IDSVKGRFTI SRDNPKNSLY
LQMSSLTAED TAVYYCARET WNHLFDYWGL GTLVTVSSAK
TTPPSVYPLA PGSAAQTNSM VTLGCLVKGY FPEPVTVTWN
SGSLSSGVHT FPAVLQSDLY TLSSSVTVPS STWPSETVTC
NVAHPASSTK VDKKIVPRDC GCKPCICTVP EVSSVFIFPP
KPKDVLTITL TPKVTCVVVD ISKDDPEVQF SWFVDDVEVH
TAQTQPREEQ FASTFRSVSE LPIMHQDWLN GKEFKCRVNS
AAFPAPIEKT ISKTKGRPKA PQVYTIPPPK EQMAKDKVSL
TCMITDFFPE DITVEWQWNG QPAENYKNTQ PIMDTDGSYF
IYSKLNVQKS NWEAGNTFTC SVLHEGLHNH HTEKSLSHS PG
(SEQ ID NO: 121)
ALTQPASVSA NPGETVKITC SGGDYYSTYY AWYQQKSPGS
APVTVIHSDD KRPSDIPSRF SGSASGSAAT LIITGVRVED
EAVYYCGGYD GRTYINTFGA GTTLTVLGQP KSSPSVTLFP
PSSEELETNK ATLVCTITDF YPGVVTVDWK VDGTPVTQGM
ETTQPSKQSN NKYMASSYLT LTARAWERHS SYSCQVTHEG
HTVEKSLSRA DCS

To prepare anti-SIRP multimer E, SEQ ID NOs: 117 and 118 were expressed in 293FS cells. The multimer was purified by standard Protein A affinity chromatography method according to manufacturer's recommended protocol (MabSelect LX, Cytiva) and dialyzed into 1× phosphate buffered saline. Anti-SIRP multimer F was prepared by expressing SEQ ID NOs: 120 and 121 is 293F cells and purifying the multimer as described for anti-SIRP multimer E.

### Example 5: Mitigating the Interference of Drugs that Comprise (i) an Antibody Fc Region and (ii) a Moiety that Binds to Human CD47 in Routine Serological Tests Using Anti-SIRP Multimer E or Anti-SIRP Multimer F

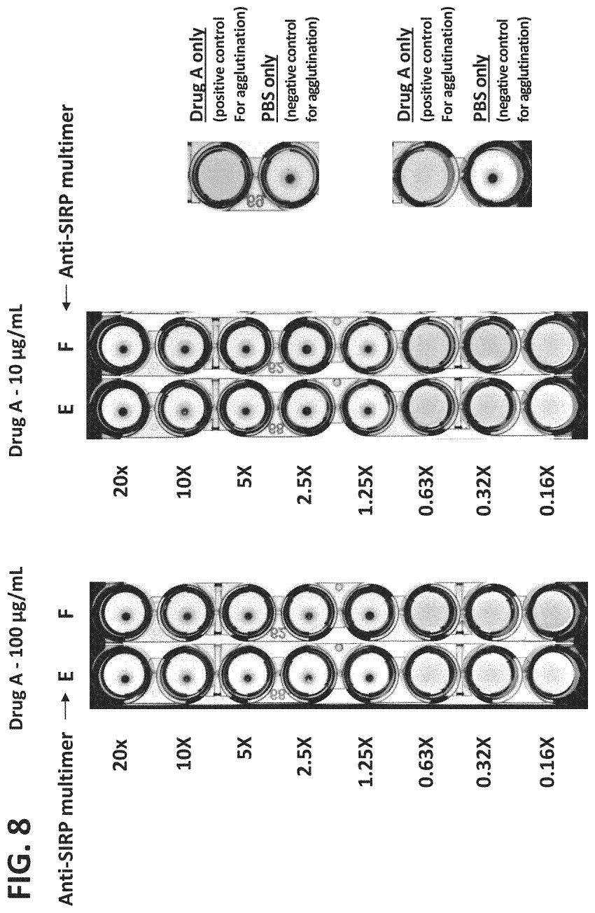

Experiments were performed to compare the degree to which anti-SIRP multimer E and anti-SIRP multimer F can mitigate the interference of Drug A in a solid phase red cell adherence assay (SPRCA). The SPRCA assay was performed as described in Example 3. As shown in FIG. 8, both anti-SIRP multimer E and anti-SIRP multimer F are able to mitigate interference of Drug A equivalently, at addition of molar ratio of at least 1.25× relative to Drug A.

### Example 6: Preparation of Exemplary Anti-SIRP Multimer G

Anti-SIRP multimer G comprises a heavy chain that comprises SEQ ID NO: 119 and a light chain that comprises SEQ ID NO: 118.

(SEQ ID NO: 119)
DVQLVESGGG VVRPGESLRL SCAASGFTFS SNAMSWVRQA
PGKGLEWLAG ISAGGSDTYY PASVKGRFTI SRDNSKNTLY
LQMNTLTAED TAVYYCARET WNHLFDYWGL GTLVTVSSAK
TTAPSVYPLA PVCGDTTGSS VTLGCLVKGY FPEPVTLTWN
SGSLSSGVHT FPAVLQSDLY TLSSSVTVTS STWPSQSITC
NVAHPASSTK VDKKIEPRGP TIKPCPPCKC PAPNLLGGPS
VFIFPPKIKD VLMISLSPIV TCVVVDVSED DPDVQISWFV
NNVEVHTAQT QTHREDYNST LRVVSALPIQ HQDWMSGKEF
KCKVNNKDLP APIERTISKP KGSVRAPQVY VLPPPEEEMT
KKQVTLTCMV TDFMPEDIYV EWTNNGKTEL NYKNTEPVLD
SDGSYFMYSK LRVEKKNWVE RNSYSCSVVH EGLHNHHTTK
SFSRTPG
(SEQ ID NO: 118)
ALTQPASVSA NPGETVKIAC SGGDYYSYYY GWYQQKAPGS
ALVTVIYSDD KRPSDIPSRF SGSASGSTAT LTITGVRAED
EAVYYCGGYD YSTYANAFGA GTTLTVLGQP KSSPSVTLFP
PSSEELETNK ATLVCTITDF YPGVVTVDWK VDGTPVTQGM
ETTQPSKQSN NKYMASSYLT LTARAWERHS SYSCQVTHEG
HTVEKSLSRA DCS

To prepare anti-SIRP multimer G, SEQ ID NOs: 118 and 119 were expressed in recombinant host cells. The multimer was purified by standard methods.

### Example 7: Drug a Interferes with Blood-Typing Assays Performed Using a Variety of Platforms

Based on previous results on Drug A's potential to interfere with RBC antibody screening (see, e.g., Kim et al. (2020) Transfusion. 1-9; doi: 10.1111/trf.16009), Drug A was spiked into normal pooled plasma that had been confirmed to be free of antibodies that bind red blood cell surface antigens. Patient plasma samples used in this study were collected as a part of routine care in a hospital setting. Final concentrations of Drug A were 0.1, 1, 10, 100, 1000, and 2000 μg/mL and tested in gel card format using Bio-Rad antibody screening cells I and II (Table 1), in solid phase using Immucor's automated, high-throughput NEO® system (Table 2), and using Diagast's automated, high-throughput Qwalys 3 system for antibody screening cells I, II and III (Table 3). As show in Tables 1-3, The agglutination reactivities ranged from 2+ to 4+(on a scale of 0 to 4+) and were observed across the Drug A concentrations tested, suggesting that the binding of Drug A to RBCs and the interaction of the Fc portion of Drug A with the AHG reagents interferes with the aforementioned assays using the aforementioned formats. Reagents were used according to manufacturer's protocols. Bio-Rad reagent information includes Antibody screening I cell and II cell: ID-DiaCell I-II; rr phenotype from ID cell: ID-DiaPanel; Gel card: ID-Card. The gel card assay was conducted according to manufacturer's protocol. Briefly, 50 μL of 0.8% RBC suspension and 25 μL of plasma were utilized; 15 min incubation at 37° C., 10 min centrifugation with Bio-Rad ID-Centrifuge. The plasma was pooled plasma from patients confirmed to be group AB and contained no alloantibodies.

TABLE 1
Results of Drug A Interference Experiments in Gel Card (Bio-Rad)

| Antibody Screening | | | | |
|---|---|---|---|---|
| | I cell | II cell | rr phenotype | |
| Spiked Drug | (0.8% R1R1, | (0.8% R2R2, | from Bio-Rad | D- - |
| A (μg/ml) | RBCs) | RBCs) | ID cell | (patient’s cell) |
| 2000.00 | 3+ | 3+ | 3+ | 3+ |
| 1000.00 | 3+ | 3+ | 3+ | 3+ |
| 100.0 | 3+ | 3+ | 3+ | 2+ |
| 10.0 | 3+ | 3+ | 3+ | 2+ |
| 1.0 | 3+ | 2+ | 3+ | 2+ |
| 0.1 | 2+ | 2+ | 2+ | 2+ |

Reagents for the NEO® solid phase platform were supplied by Immucor for routine use with the NEO® system. Reagent included Capture-R Ready-Screen (I and II). Reagent red cells (in the form of red cell stroma) were bound to test wells at the time of manufacture. Plasma volume per well was 25 μL.

TABLE 2
Results of Drug A Interference Experiments in
NEO ® Solid Phase Platform.
Antibody Screening using Solid Phase Testing

| Spiked Drug | I (R1R1, | II (R2R2, |
|---|---|---|
| A (μg/ml) | coated RBCs) | coated RBCs) |
| 2000.00 | 4+ | 4+ |
| 1000.00 | 4+ | 4+ |
| 100.0 | 4+ | 4+ |
| 10.0 | 4+ | 4+ |
| 1.0 | 4+ | 4+ |
| 0.1 | 4+ | 4+ |

Routine reagents used with the QWALYS 3 platform (Erythrocyte Magnetized Technology (EMT)) were supplied by Diagast. One reagent was Hemascreen (1% magnetized red cells). Red cell and plasma volume per well were 15 and 15 μL, respectively.

TABLE 3
Results of Drug A Interference Experiments in QWALYS 3 Platform.
Antibody Screening

| | I (R1R1, | II (R2R2, | III (rr, |
|---|---|---|---|
| Spiked Drug A | magnetized | magnetized | magnetized |
| (μg/ml) | RBCs) | RBCs) | RBCs) |
| 2000.00 | 4+ | 4+ | 4+ |
| 1000.00 | 4+ | 4+ | 4+ |
| 100.0 | 4+ | 4+ | 4+ |
| 10.0 | 4+ | 4+ | 4+ |
| 1.0 | 4+ | 4+ | 4+ |
| 0.1 | 4+ | 4+ | 4+ |

The data in Table 1 show that Drug A caused 2+ to 3+ agglutination reactivity in gel card testing (Bio-Rad). Comparable agglutination reactivity was observed in gel card testing using D—, i.e., RBCs with significantly decreased CD47 expression, as well as in gel card using rr, i.e., RBCs with high CD47 expression. The data in Tables 2 and 3 show that Drug A caused 4+agglutination reactivity in high-throughput solid phase testing (NEO®, QWALYS®), even at very low concentrations of Drug A.

### Example 8: Mitigating the Interference of Drugs that Comprise (i) an Antibody Fc Region and (ii) a Moiety that Binds to Human CD47 in Routine Serological Tests Using Anti-SIRP Multimer G

Experiments were performed to assess the degree to which anti-SIRP multimer G mitigates the interference of Drug A in a gel card assay. Assays were performed as described in Example 7. In one series of tests, Multimer G was added to plasma samples to achieve a final concentration of 0.1, 1, 10, 100, 1000, or 2000 μg/mL. Gel card serological assays were performed. As shown in Table 4, a 6-fold molar excess of anti-SIRP multimer G relative to Drug A mitigated interference of Drug A when Drug A was present in the sample at a concentration of 2000 μg/mL. A 4-fold molar excess of anti-SIRP multimer G relative to Drug A mitigated interference of Drug A when Drug A was present in the sample at a concentration of 1000 μg/mL. A 3-fold molar excess of anti-SIRP multimer G mitigated the interference of Drug A when Drug A present in the sample at concentrations between 0.1 and 100 ng/mL.

TABLE 4
Mitigation of Interference by Drug A in Gel Card using Multimer G

| Spiked | Molar Excess of Multimer G | | | | | | | | | | | | | | | | | |
|---|---|---|---|---|---|---|---|---|---|---|---|---|---|---|---|---|---|---|
| Drug A con | x1 | | | x2 | | | x3 | | | x4 | | | x5 | | | x6 | | |
| (μg/mL) | I | II | III | I | II | III | I | II | III | I | II | III | I | II | III | I | II | III |
| 2000.0 | 1+ | 1+ | 1+ | 0.5+ | 0.5+ | 1+ | 0.5+ | 0.5+ | 0.5+ | 0.5+ | 0.5+ | 0.5+ | 0.5+ | 0.5+ | 0.5+ | 0 | 0 | 0 |
| 1000.0 | 0.5+ | 0.5+ | 0.5+ | 0.5+ | 0.5+ | 0.5+ | 0.5+ | 0.5+ | 0.5+ | 0 | 0 | 0 | 0 | 0 | 0 | 0 | 0 | 0 |
| 100.0 | 2+ | 2+ | 2+ | 0.5+ | 0.5+ | 0.5+ | 0 | 0 | 0 | 0 | 0 | 0 | 0 | 0 | 0 | 0 | 0 | 0 |
| 10.0 | 2+ | 2+ | 2+ | 0 | 0 | 0.5+ | 0 | 0 | 0 | 0 | 0 | 0 | 0 | 0 | 0 | 0 | 0 | 0 |
| 1.0 | 2+ | 1+ | 2+ | 0 | 0 | 0.5+ | 0 | 0 | 0 | 0 | 0 | 0 | 0 | 0 | 0 | 0 | 0 | 0 |
| 0.1 | 1+ | 1+ | 1+ | 0 | 0 | 0.5+ | 0 | 0 | 0 | 0 | 0 | 0 | 0 | 0 | 0 | 0 | 0 | 0 |

I: 0.8% R1R1 RBC;
II: 0.8% R2R2 RBC;
III: 0.8% rr RBC. RBC and gel card were purchased from Bio-Rad.

Next, Drug A was added to plasma samples containing anti-Jka or anti-E antibodies (i.e., known alloantibodies) to achieve a final Drug A concentration of 0.1, 1, 10, 100, 1000, or 2000 μg/mL. Gel Gard assays were performed. As shown in Table 5, Drug A, present in the plasma at concentration of 2000 μg/mL, interfered with the detection anti-Jka and anti-E alloantibodies. Multimer G neutralized the interference of Drug A without interfering with the detection of the anti-Jka and anti-E alloantibodies.

TABLE 5
Mitigation of Interference by Drug A in Gel Card using Multimer G

| | I | II | III |
|---|---|---|---|
| Spiked Drug | (R1R1, | (R2R2, | (rr, |
| A con (μg/mL) | Jka− Jkb+) | Jka+ Jkb−) | Jka+ Jkb−) |
| Only anti-E | 0 | 1+ | 0 |
| Only anti-Jka | 0 | 1+ | 1+ |
| Drug A 2000.0 + Anti-E | 3+ | 3+ | 3+ |
| Drug A 2000.0 + Anti-Jka | 3+ | 3+ | 3+ |
| Drug A 2000.0 + | 0 | 1+ | 0 |
| Anti-E + Multimer G ×6 | | | |
| Drug A 2000.0 + Anti-Jka + | 0 | 1+ | 1+ |
| Multimer G ×6 | | | |

I: 0.8% R1R1 RBC;
II: 0.8% R2R2 RBC;
III: 0.8% rr RBC. RBC and gel card were purchased from Bio-Rad.

The data in Tables 4 and 5 support the conclusion that anti-SIRP Multimer G mitigates the interference caused by Drug A present in plasma. Such mitigation permits the detection of alloantibodies in the plasma via gel card assays.

### Example 9: The Interference in Serological Assays Caused by Drugs that Comprise (i) an Antibody Fc Region and (ii) a Moiety that Binds to Human CD47 can Mitigated Using Multimer G Across a Variety of Platforms

Further tests were performed to assess the degree to which Drug A interferes with serological assays in tube test and gel card formats. Plasma samples (AB inert plasma) were spiked with Drug A to achieve a final Drug A concentration of 31.25, 125, 500 or 2000 μg/ml. In tube tests, the presence of Drug A in plasma at concentrations of 500 μg/ml and 2000 μg/ml caused a strong positive direct antiglobulin test (DAT). Drug A at 31.25 μg/ml and 2000 μg/ml concentrations tested did not interfere with ABO forward and reverse typing or with RhD typing. Interference was observed in RhD typing for weak D at AHG due to the positive DAT. Drug A at all concentrations tested (31.25 μg/ml, 125 μg/ml, 500 μg/ml and 2000 μg/ml) did not interfere with antibody screen at initial spin (where traditional ABO incompatibility was being tested) but caused strong agglutination with all cells in indirect antiglobulin testing (IAT) using PEG and both AHG reagents tested (Immucor and Ortho). In the gel card testing, Drug A caused strong positive agglutination with all tested cells at all concentrations of Drug A (i.e., 31.25 μg/ml, 125 μg/ml μg/ml, 500 μg/ml and 2000 μg/ml). (Data not shown.)

Multimer G (100 mg/ml), when added to plasma at a ratio of 1:10 (v/v, Multimer G:plasma) and incubated for 15 minutes at room temperature, eliminated interference of Drug A present in the plasma at a concentration of 500 μg/ml. See Table 6A. Multimer G (100 mg/ml), when added to plasma at a ratio of 1:10 (v/v, Multimer G:plasma) and incubated for up to 60 minutes at room temperature or at 37° C., partially mitigated interference by Drug A (“vw+”, or “very weak+,” which falls between 0 and 1+), when present in plasma at a concentration of 2000 μg/ml, but antibody screen remained positive (“w1+” or “weak 1+,” which falls between 0 and 1+). See Table 6B. Reactivity in the antibody screen was greatly diminished, but remained weakly positive, in testing by PEG-IAT, and LISS-IAT. See Table 6C.

TABLE 6A
Neutralization and 3 cell antibody screen−15 min at Room Temp.

| Drug A 500 μg/mL | | | | Drug A | |
|---|---|---|---|---|---|
| with Multimer G | | | | alone | plasma |
| RBC | R1R1 | R2R2 | rr | R1R1 | R1R1 |
| sample | | | | | |
| Gel Card | 0 | 0 | 0 | 3+ | 0 |
| score | | | | | |

TABLE 6B
Neutralization and 3 cell antibody screen-60 min at Room Temp

| | | | | Drug A | |
|---|---|---|---|---|---|
| Drug A 2000 μg/mL with Multimer G | | | | alone | plasma |
| RBC | R1R1 | R2R2 | rr | R1R1 | R1R1 |
| sample | | | | | |
| Gel Car | vw+ | vw+ | w1+ | 3+ | 0 |
| score | | | | | |

TABLE 6C
Neutralization Study in IAT format

| | Drug A 2000 μg/ml | | |
|---|---|---|---|
| | with rr RBCs | | |
| | Without | With | Plasma |
| | Multimer | Multimer | Only |
| | G | G | NA |
| LISS | 12 | 4-5 (1+) | 0 |
| PEG | 11 | 4-5 (1+) | 0 |

The use of Multimer G did not affect reactivity of alloanti-D by LISS or PEG IAT or by the gel card test. See Table 7. The use of Multimer G did not affect reactivity of alloanti-K by LISS or PEG IAT or by the gel card test. See Table 8.

TABLE 7
Results of Alloanti-D Testing*

| | | | | 1500 μg/ml Drug |
|---|---|---|---|---|
| | | | 1500 μg/ml Drug | A + Anti-D + |
| Test Format | Cell | Anti D | A + Anti-D | Multimer G |
| LISS-IAT | D+ | 11 | 11 | 11 |
| | D− | 0 | 10 | 0 |
| PEG-IAT | D+ | 9 | 11 | 9 |
| | D− | 0 | 9 | 0 |
| Gel Card | D+ | 3+ | 3+ | 3+ |
| | D− | 0 | 2+ | 0 |

*Scoring ranges between 0-12, with 12 indicated highest reactivity

TABLE 8
Results of Alloanti-K Testing*

| | | | | 1500 μg/ml Drug |
|---|---|---|---|---|
| | | | 1500 μg/ml Drug | A + Anti-K + |
| Test Format | Cell | Anti-K | A + Anti-K | Multimer G |
| LISS-IAT | K+ | 11 | 12 | 11 |
| | K− | 0 | 11 | 0 |
| PEG-IAT | K+ | 10 | 12 | 11 |
| | K− | 0 | 11 | 0 |
| Gel Card | K+ | 2+ | 3+ | 2+ |
| | K− | 0 | 2+ | 0 |

*Scoring ranges between 0-12, with 12 indicated highest reactivity

As shown in Table 9A, Drug A causes agglutination in LISS IAT, PEG IAT, and gel card test formats.

TABLE 9A
Drug A reactivity in Serological Assays

| | | | | | | Controls | |
|---|---|---|---|---|---|---|---|
| | Drug A Concentration in AB Inert plasma (μg/mL) | | | | | AB | AB Inert: |
| | 750 | 1000 | 1250 | 1500 | 2000 | Inert | Buffer (1:10) |
| LISS IAT | 3+^{s} | 4+ | 4+ | 3+^{s} | 3+^{s} | 0 | 0 |
| PEG IAT | 4+ | 3+^{s} | 3+^{s} | 3+^{s} | 3+^{s} | 0 | 0 |
| Gel Card | 3+ | 3+ | 3+ | 3+ | 3+ | 0* | 0 |
| Test | | | | | | | |

*RBC membrane layer observed at top of AB inert control; 3+^{s} = “strong 3+”

The following results are presented in Table 9B: Multimer G (150 mg/ml) was added to plasma at a ratio of 1:10 (v/v, Multimer G:plasma). After 30 minutes of incubation, interference by Drug A, when present in plasma at 750 μg/ml or 1000 μg/mL, was fully mitigated by Multimer G in the LISS IAT test. After 30 minutes of incubation, interference by Drug A, when present in plasma at 1250 μg/ml, 1500 μg/ml, or 2000 μg/mL, was virtually abolished (micro+ reactivity) by Multimer G in the LISS IAT test. After 30 minutes of incubation, interference by Drug A at all concentrations tested (750 μg/ml, 1000 μg/mL, 1250 μg/mL, 1500 μg/mL, and 2000 μg/mL) was greatly diminished (1+ or micro+) by Multimer G in the PEG IAT test. After 30 minutes of incubation, interference by Drug A, when present in plasma at 750 μg/ml, 1000 μg/mL, or 1250 μg/mL, was fully mitigated by Multimer Gin the IgG gel test. After 30 minutes of incubation, interference by Drug A, when present in plasma at 1500 ng/ml or 2000 μg/mL, was greatly diminished 1+ or +/−) by Multimer Gin the IgG gel test.

TABLE 9B
Neutralization Assays-30 minute

| | Drug A Concentration in AB Inert Plasma* (μg/mL) | | | | |
|---|---|---|---|---|---|
| | 750 | 1000 | 1250 | 1500 | 2000 |
| LISS (Neut. 1:10 | 0 | 0 | Micro+ | Micro+ | Micro+ |
| Multimer G; 30 min) | | | | | |
| PEG (Neut. 1:10 | Micro+ | Micro+ | Micro+ | Micro+ | 1+ |
| Multimer G; 30 min.) | | | | | |
| Gel Card Test | 0 | 0 | 0 | (+/-) | 1+ |

*AB inert plasma is used as negative control for IAT tests

The following results are presented in Table 9C. Multimer G (150 mg/ml) was added to plasma at a ratio of 1:10 (v/v, Multimer G:plasma). After 60 minutes of incubation, interference by Drug A, when present in plasma at 750 μg/ml, 1000 μg/mL, or 1250 μg/mL, was fully mitigated by Multimer G in the LISS IAT test. After 60 minutes of incubation, interference by Drug A, when present in plasma at 1500 μg/mL, was virtually abolished (micro+reactivity) by Multimer G in the LISS IAT test. Mitigation of interference by Drug A, when present in plasma at 2000 μg/mL, by Multimer G was not assessed in the LISS IAT test in this experiment. After 60 minutes of incubation, interference by Drug A, when present in plasma at 750 μg/mL, 1000 μg/mL, 1250 μg/mL, or 1500 μg/mL, was virtually abolished (micro+reactivity) by Multimer G in the PEG IAT test. Mitigation of interference by Drug A, when present in plasma at 2000 μg/mL, by Multimer G was not assessed in the PEG IAT test in this experiment. After 60 minutes of incubation, interference by Drug A, when present in plasma at 750 ng/ml or 1000 μg/mL, was fully mitigated by Multimer G, and when present in plasma at 1250 μg/mL or 1500 μg/mL, was greatly diminished (weak+ or +/−) by Multimer Gin the IgG gel test. Mitigation of interference by Drug A, when present in plasma at 2000 μg/mL, by Multimer G was not assessed in the IgG gel test.

TABLE 9C
Neutralization Assays-60 minute

| | Drug A Concentration in AB Inert (μg/mL) | | | | |
|---|---|---|---|---|---|
| | 750 | 1000 | 1250 | 1500 | 2000 |
| LISS (Neut. 1:10 | 0 | 0 | 0 | Micro+ | NT* |
| Multimer G) | | | | | |
| PEG (Neut. 1:10 | Micro+ | Micro+ | Micro+ | Micro+ | NT* |
| Multimer G) | | | | | |
| Gel Card Test (Neut. | 0 | 0 | (+/-) | w+ | NT* |
| 1:10 Multimer G) | | | | | |

*NT = not tested

1. A method of reducing drug interference in a serological assay using reagent red blood cells (RBC) or reagent platelets, said method comprising:
(a) adding a CD47 multimer that binds to the drug and blocks the drug from binding the reagent RBC or the reagent platelets to a plasma sample from a subject who has received treatment with the drug; and
(b) performing the serological assay of the plasma sample after step (a), using the reagent RBC or the reagent platelets,
wherein the drug comprises (i) a human antibody Fc region or variant thereof and (ii) a moiety that binds to human CD47, and
wherein the CD47 multimer comprises at least two CD47 polypeptide monomers.

2-65. (canceled)

66. A method of reducing drug interference in a serological assay using reagent red blood cells (RBC) or reagent platelets, said method comprising:
(a) adding an anti-SIRP multimer that binds to the drug and blocks the drug from binding the reagent RBC or the reagent platelets to a plasma sample from a subject who has received treatment with the drug; and
(b) performing the serological assay of the plasma sample after step (a), using the reagent RBC or the reagent platelets,
wherein the drug comprises (i) a human antibody Fc region or variant thereof and (ii) a moiety that binds to human CD47, and
wherein the anti-SIRP multimer comprises one or more anti-SIRP antibodies or drug-binding fragments thereof.

67. The method of claim 66, wherein the anti-SIRP multimer comprises between 1 and 100 anti-SIRP antibodies or drug-binding fragments thereof.

68. The method of claim 66, wherein the anti-SIRP multimer comprises an anti-SIRP antibody or drug-binding fragment thereof that binds to a wild type SIRPα, a SIRPα variant, a SIRPβ variant, a wild-type SIRPγ, a SIRPγ variant, or any two or more of the preceding.

69. The method of claim 66, wherein the anti-SIRP multimer comprises an anti-SIRP antibody or drug-binding fragment thereof that comprises:
(a) a heavy chain variable domain (V_{H}) that comprises SEQ ID NO: 46 and a light chain variable domain (V_{L}) that comprises SEQ ID NO: 47;
(b) a heavy chain variable domain (V_{H}) that comprises SEQ ID NO: 48 and a light chain variable domain (V_{L}) that comprises SEQ ID NO: 49;
(c) a heavy chain variable domain (V_{H}) that comprises SEQ ID NO: 50 and a light chain variable domain (V_{L}) that comprises SEQ ID NO: 51;
(d) a heavy chain variable domain (V_{H}) that comprises SEQ ID NO: 113 and a light chain variable domain (V_{L}) that comprises SEQ ID NO: 114;
(e) a heavy chain variable domain (V_{H}) that comprises SEQ ID NO: 115 and a light chain variable domain (V_{L}) that comprises SEQ ID NO: 116; and/or
(f) a heavy chain variable domain (V_{H}) that comprises SEQ ID NO: 133 and a light chain variable domain (V_{L}) that comprises SEQ ID NO: 134.

70. The method of claim 66 wherein the anti-SIRP multimer comprises a full length anti-SIRP antibody.

71. The method of claim 70, wherein the anti-SIRP antibody comprises a murine Fc domain.

72. The method of claim 71, wherein the murine Fc domain comprises an amino acid sequence set forth in any one of SEQ ID NOs: 81-83.

73. The method of claim 70, wherein the anti-SIRP antibody comprises:
(a) a heavy chain that comprises SEQ ID NO: 117 and a light chain that comprises SEQ ID NO: 118;
(b) a heavy chain that comprises SEQ ID NO: 119 and a light chain that comprises SEQ ID NO: 118;
(c) a heavy chain that comprises SEQ ID NO: 120 and a light chain that comprises SEQ ID NO: 121; or
(d) a heavy chain that comprises SEQ ID NO: 122 and a light chain that comprises SEQ ID NO: 121.

74. The method of claim 66, wherein the drug-binding fragment of the anti-SIRP antibody is a Fab, a Fab′, an F(ab′)₂, a Fab′-SH, an Fv, a diabody, a one-armed antibody, an scFv, an scFv-Fc, a single domain antibody, or a single heavy chain antibody.

75. The method of claim 74, wherein the drug binding fragment comprises a F(ab′)2, and wherein the F(ab′)2 comprises SEQ ID NOs: 131 and 132.

76. The method of claim 66, wherein the anti-SIRP antibody or drug-binding fragment thereof comprises an epitope tag or a ligand.

77. The method of claim 76, wherein the epitope tag comprises any one of SEQ ID NOs: 7-32 and 126, or wherein the ligand comprises biotin.

78. The method of claim 76, wherein the epitope tag comprises HHHHHHGLNDIFEAQKIEWHE (SEQ ID NO: 135) or GSGSHHHHHHGLNDIFEAQKIEWHE (SEQ ID NO: 126).

79. The method of claim 66, wherein the one or more anti-SIRP antibodies or drug-binding fragments thereof are attached to a solid support.

80. (canceled)

81. The method of claim 79, wherein each of the one or more anti-SIRP antibodies or drug-binding fragments thereof comprises an epitope tag or a ligand, wherein a capture agent that specifically binds the epitope tag or ligand is immobilized on the solid support, and wherein the anti-SIRP antibodies or drug-binding fragments thereof are attached to the solid support by the specific binding of the epitope tag or ligand by the capture agent.

82. The method of claim 81, wherein the ligand is biotin and wherein the capture agent is streptavidin.

83. The method of claim 66, wherein the anti-SIRP multimer comprises a streptavidin or avidin bound to 2, 3, or 4 biotinylated anti-SIRP antibodies or fragments thereof.

84. The method of claim 82, wherein the anti-SIRP multimer comprises the streptavidin or the avidin bound to 2, 3, or 4 biotinylated F(ab′)2 fragments, wherein 2 or more of the biotinylated F(ab′)2 fragments comprise SEQ ID NOs: 131 and 132.

85. The method of claim 66, wherein the anti-SIRP multimer is a homomultimer or a heteromultimer.

86-87. (canceled)

88. The method of claim 66, wherein the moiety of the drug that binds to human CD47 comprises:
(a) a wild type SIRPα,
(b) a SIRPα variant that comprises one or more amino acid substitution(s), insertion(s), deletion(s), N-terminal extension(s), and/or C-terminal extension(s) relative to the wild type SIRPα,
(c) a fragment of the wild type SIRPα that comprises an extracellular domain of the wild type SIRPα, or
(d) a fragment of the SIRPα variant that comprises an extracellular domain of the SIRPα variant.

89-90. (canceled)

91. The method of claim 66, wherein the moiety of the drug that binds to human CD47 comprises:
(a) a wild type SIRPγ,
(b) a SIRPγ variant that comprises one or more amino acid substitution(s), insertion(s), deletion(s), N-terminal extension(s), and/or C-terminal extension(s) relative to the wild type SIRPγ,
(c) a fragment of the wild type SIRPγ that comprises an extracellular domain of the wild type SIRPγ, or
(d) a fragment of the SIRPγ variant that comprises an extracellular domain of the SIRPγ variant.

92-93. (canceled)

94. The method of claim 66, wherein the moiety of the drug that binds to human CD47 comprises:
(a) a SIRPβ variant that comprises one or more amino acid substitution(s), insertion(s), deletion(s), N-terminal extension(s), C-terminal extension(s), or any combination of the preceding, relative to the wild type SIRPβ, or
(b) a fragment of the SIRPβ variant that comprises an extracellular domain of the SIRPβ variant.

95-96. (canceled)

97. The method of claim 66, wherein the antibody Fc region of the drug is a human IgG Fc region, and wherein the human IgG Fc region is IgG1, IgG2, IgG4, or a variant of any one of the preceding.

98. (canceled)

99. The method of claim 66, wherein the serological assay is:
(a) an ABO/Rh typing assay,
(b) an immediate spin (IS) assay,
(c) a direct antiglobulin (DAT) assay using a polyspecific reagent that detects IgG and complement C3,
(d) a direct antiglobulin (DAT) assay using a monospecific reagent that detects complement C3,
(e) a PEG-enhanced serological assay,
(f) an eluate test that is performed following a DAT assay,
(g) a tube assay or a solid phase red cell assay (SPRCA),
(h) a gel card assay, or
(i) a solid phase assay.

100-170. (canceled)

171. An anti-SIRP multimer comprising one or more anti-SIRP antibodies or drug-binding fragments thereof.

172-191. (canceled)
